SEQUENCE LISTING (1 sequence)

SEQ ID NO: 1 - DNA, 22 bp, synthetic construct
  source 1..22: mol_type = other DNA
  modified_base 1..22: mod_base = OTHER; note = phosphorothioate bond
tcgtcgttttcggcgcgcgccg

(19) United States
(12) Patent Application Publication
(10) Pub. No.: US 2024/0299454 A1
(43) Pub. Date: Sep. 12, 2024

(54) ANTIGEN-AGNOSTIC COMBINATION IMMUNOTHERAPY

(71) Applicant: UNIVERSITÄT ZU KÖLN, Köln (DE)

(72) Inventors: Sven BORCHMANN, Köln (DE); Roland ULLRICH, Köln (DE)

(21) Appl. No.: 18/582,076

(22) Filed: Feb. 20, 2024

Related U.S. Application Data
(63) Continuation of application No. PCT/EP22/72913, filed on Aug. 17, 2022.

(30) Foreign Application Priority Data
Aug. 18, 2021 (EP) 21191841.2

(51) Int. Cl. A61K 35/17; A61K 38/20; A61K 39/00; A61K 45/06; A61P 35/00; C12N 5/0783

(52) U.S. Cl. CPC A61K 35/17; A61K 38/2013; A61K 39/4611; A61K 45/06; A61P 35/00; C12N 5/0638; A61K 2239/30; A61K 2239/38

(57) ABSTRACT

The present invention relates to a medicament for use in a method of preventing or treating cancer in a patient, wherein the medicament comprises at least two of the following populations of cells (i) to (iv): (i) a population of lymphokine-activated killer cells (LAKs), (ii) a population of cytokine-induced killer cells (CIKs), (iii) a population of γδ-T-cells, (iv) a population of tumor-specific T-cells (CTLs), wherein the population of cells in (i) to (iv) are derived from autologous cells from said patient or from allogeneic cells from a donor, and pharmaceutical composition, kit or kit-of-parts related thereto.

### CROSS-REFERENCE TO RELATED APPLICATIONS

This application is a bypass continuation of International Application No. PCT/EP2022/072913, filed Aug. 17, 2022, which claims the benefit of priority application EP 21191841.2, filed Aug. 18, 2021, the content of each of which is herein incorporated by reference.

### SEQUENCE LISTING

The instant application contains a sequence Listing which has been submitted electronically in XML file format and is hereby incorporated by reference in its entirety. Said XML copy, created on Aug. 4, 2022, is named P73811PC.xml and is 3 kilobytes in size.

### TECHNICAL FIELD

The present invention relates to the technical field of immunology and cancer therapy. Provided herein are medicaments, comprising different populations of immune cells for use in a method of preventing and/or treating cancer in a patient as well as pharmaceutical compositions, kits and kit-of parts related thereto.

### BACKGROUND

Cancer is a leading cause of morbidity and mortality¹. While surgery, chemo- and radiotherapy have been the core elements of cancer treatment throughout much of its history, immunotherapy has only recently been added to this repertoire²³. Single-agent immunotherapy, such as immune checkpoint inhibition, has shown remarkable efficacy in some cancers and led to durable remissions in selected patients⁴. However, diverse immunosuppressive mechanisms act in a concerted way to suppress the host anti-tumor immune response. Thus, single-agent immunotherapy is unlikely to be sufficient to overcome these mechanisms⁵, and indeed most human cancers do not respond to single-agent immunotherapy⁶. In general, tumors responsive to immunotherapy, such as immune checkpoint blockade, have a permissive tumor immune microenvironment (TIME) that is characterized by already existing immune infiltration, active antigen-presentation, and immunogenic cell death⁵. In contrast, poorly inflamed tumors, so called immune-deserts or cold tumors, do mostly not respond to immunotherapy⁷.

While some combination immunotherapies have been shown to be effective in preclinical models of such cold tumors, they rely on knowledge of antigenic determinants such as tumor-associated- or neo-antigens⁸. However, many tumors express few of those and even if present, these are mostly not known for a given patient. Until now, most combination immunotherapy approaches that are effective in poorly immunogenic tumors rely on precise knowledge of the antigenic determinants of a tumor and include adoptively transferred cells, vaccinations or antibodies engineered to directly target those^{8,50}. Thus, creating an antigen-agnostic combination immunotherapy that is effective in poorly immunogenic tumors for which an antigenic determinant is not known is a major challenge.

Thus, it is an object of the present invention to overcome present limitations of current immune therapies that e.g. rely on antigen-specific approaches, for patients suffering from cancer. Therefore, it is another object of the present invention to provide a medicament, comprising different cell populations for use in a method of preventing or treating cancer, which can be applied in a versatile and uncomplicated way. It is a further object of the present invention to provide a pharmaceutical composition, a kit or a kit-of-parts comprising these different cell populations which are suitable for uses and methods as described above.

### SUMMARY OF THE INVENTION

The above-mentioned objects have been solved by the aspects of the present invention as specified hereinafter.

Accordingly, in a first aspect of a present invention provided herein is a medicament for use in a method of preventing or treating cancer in a patient, wherein the medicament comprises at least two of the following populations of cells (i) to (iv):
(i) a population of lymphokine-activated killer cells (LAKs),
(ii) a population of cytokine-induced killer cells (CIKs),
(iii) a population of γδ-T-cells,
(iv) a population of tumor-specific T-cells (CTLs), wherein the population of cells in (i) to (iv) are derived from autologous cells from said patient or from allogeneic cells from a donor.

According to a preferred embodiment, the medicament comprises at least three of the populations of cells (i) to (iv) or comprises the populations of cells of (i), (ii), (iii) and (iv).

According to another preferred embodiment, (a) the autologous cells from said patient or from allogeneic cells from a donor are cells from a blood sample, preferably a PBMC sample, and/or (b) the population of cells in (i) to (iv) are enriched and/or generated by cultivation in vitro, and/or (c) the population of tumor-specific T-cells is enriched from the autologous cells or from allogeneic cells by co-cultivating the autologous cells or the allogeneic cells with tumor cells from said patient.

According to another preferred embodiment, the method further comprises administering to the patient one or more of the following (v) to (viii): (v) at least one Th-1 adjuvant, (vi) at least one IL-2 family cytokine, (vii) at least one immune checkpoint targeting agent, (viii) at least one lymphodepleting agent.

According to a second aspect of the present invention, it is provided a medicament for use in a method of preventing or treating cancer in a patient, wherein the medicament comprises at least one of the following populations of cells (i) to (iv): (i) a population of lymphokine-activated killer cells (LAKs), (ii) a population of cytokine-induced killer cells (CIKs), (iii) a population of γδ-T-cells, (iv) a population of tumor-specific T-cells (CTLs), wherein the cells in (i) to (iv) are derived from autologous cells from said patient or from allogeneic cells from a donor, and wherein the method comprises administering to the patient: the at least one of the populations of cells of (i) to (iv), (v) the at least one Th-1 adjuvant, (vi) the at least one IL-2 family cytokine, (vii) the at least one immune checkpoint targeting agent, and (viii) the at least one lymphodepleting agent.

According to one embodiment of the present invention, (a) the populations of cells of (i) to (iv) are administered to said patient in a single composition, simultaneously or within 24 hours, and/or (b) the populations of cells of (i) to (iv) are administered in a ratio of number of cells (i):(ii):(iii):(iv) of about 0.1-10:0.1-10:0.1-10:0.1-10, and/or (c) the populations of cells of (i) to (iv) are administered systemically or locally.

According to another embodiment of the present invention, the populations of cells of (i) to (iv) are administered in a ratio of number of cells of (i):(ii):(iii):(iv) of about 1:1:1:1.

According to a further embodiment of the present invention, (a) the at least one Th-1 adjuvant is an adjuvant which is a danger signal and which is preferably selected from a STING agonist, a RIG-1 agonist, an MD5 agonist, a TLR2 agonist, a TLR3 agonist, a TLR3 agonists, a TLR4 agonist, a TLR5 agonists, a TLR7 agonist, a TLR8 agonist, a TLR9 agonist, or a combination thereof, and/or (b) the at least one Th-1 adjuvant is administered once or repeatedly to the patient, (c) the at least one Th-1 adjuvant is administered systemically or locally, preferably by inhalation, subcutaneously, by injection, intravenously or intratumorally.

In another embodiment of the present invention, (a) the at least one immune checkpoint targeting agent is selected for an antagonist anti-PD-1 antibody, an antagonist anti-PD-L1 antibody, an antagonist anti-PD-L2 antibody, an antagonist anti-CTLA-4 antibody, an antagonist anti-TIM-3 antibody, an antagonist anti-LAG-3 antibody, an antagonist anti-CEACAM1 antibody, an antagonist anti-TIGIT antibody, an agonist anti-CD137 antibody, an agonist anti-ICOS antibody, an agonist anti-GITR antibody, an agonist anti-OX40 antibody or an inhibitor of indoleamine-2,3-dioxygenase (IDO), and/or (b) the at least one immune checkpoint targeting agent is administered to the patient on the same day as the populations of cells of (i) to (iv), and/or after said day, and/or (c) the at least one immune checkpoint targeting agent is administered to the patient repeatedly.

According to an another embodiment of the present invention, (a) the at least one lymphodepleting agent is administered at least one day prior to the day on which the populations of cells of (i) to (iv) are administered, and/or (b) the at least one lymphodepleting agent is selected from cyclophosphamide, fludarabine or a combination thereof, and/or (c) the at least one IL-2 family cytokine is selected from mature human IL-2 or a fusion protein thereof, and/or (d) the at least one IL-2 family cytokine is administered to the patient on the same day as the populations of cells of (i) to (iv), and/or after said day, and/or (e) the at least one IL-2 family cytokine is administered repeatedly.

According to an another embodiment of the present invention, (a) the cancer is selected from melanoma, renal cancer, prostate cancer, breast cancer, colon cancer, lung cancer, bone cancer, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioma, pituitary adenoma, Kaposi's sarcoma, epidermoid cancer, squamous cell cancer, T-cell lymphoma, environmentally induced cancers including those induced by asbestos, esophageal cancer, liver cancer, refractory or recurrent malignancies, metastatic cancers, and combinations of said cancers, and/or (b) the tumor(s) is/are poorly immunogenic, and/or (c) the patient is human.

According to a third aspect of the present invention, provided herein is a pharmaceutical composition, kit or kit-of parts comprising at least two of the following populations of cells (i) to (iv): (i) a population of lymphokine-activated killer cells (LAKs), (ii) a population of cytokine-induced killer cells (CIKs), (iii) a population of γδ-T-cells, (iv) a population of tumor-specific T-cells (CTLs), wherein the cells in (i) to (iv) are derived from autologous cells from a patient or from allogenic cells from a donor.

According to one embodiment of the third aspect the pharmaceutical composition, kit or kit-of parts comprises three or four of (i) to (iv), optionally wherein the pharmaceutical composition, kit or kit-of parts further comprises one or more of the following: (v) at least one Th-1 adjuvant, (vi) at least one IL-2 family cytokine, (vii) at least one immune checkpoint targeting agent, (viii) at least one lymphodepleting agent.

According to another embodiment of the third aspect, (a) the at least two populations of cells are comprised in a single composition or in separate compositions, and/or (b) the at least two populations of cells are in a single container or in separate containers, and/or (c) the at least two populations of cells are comprised in a pharmaceutical composition further comprising at least one pharmaceutically acceptable excipient, and/or (d) the at least two populations of cells are in a buffered solution, and/or (e) the patient is a patient suffering from cancer.

According to another embodiment of the present invention, the pharmaceutical composition, kit or kit-of parts are further characterized by the features of the first aspect of the invention.

The invention is further detailed by the following figures:

### DESCRIPTIONS OF THE FIGURES

FIG. 1: Combined treatment with lymphokine-activated killer cells (LAKs), cytokine-induced killer cells (CIKs), Vγ9Vδ2-T-cells (γδ-T-cells) and adaptive, tumor-specific T-cells (CTLs) is superior to single cell type adoptive cellular therapy. (A) heatmap of Chou-Talalay combination index (lower equals more synergy) of various adoptive cellular therapy (ACT) subcomponents in two murine cell lines and their median, (B) experimental overview of in vivo experimental workflow for this figure, (C) mean fold change of subcutaneous B16F10 melanoma tumor volumes in C57BL/6J mice over time treated with ACT subcomponents or combined ACT at equivalent doses (n=8 per group), (D) immune cell deconvolution analysis showing mean gene expression z-scores of immune cell specific transcripts (see methods for details), (E) mean gene expression z-scores of intratumoral cyto- and chemokines (n=3-8 per group, B16F10 tumors harvested on day 21 treated in different experimental groups for (D-E), (F) heatmap of mean z-scores of cytokines quantified by multiplex Luminex analysis in the sera of B16F10 bearing C57BL/6J mice sacrificed on day 21 treated in different experimental groups, (G) volcano plot showing cytokines detected at significantly higher levels in combined ACT compared to pooled ACT subcomponent treated B16F10 melanoma bearing C57BL/6J mice, (H) heatmap of Chou-Talalay combination index of various ACT subcomponents in seven human cell lines and their median, (I) mean fold change of subcutaneous H1975 lung cancer tumor volumes in humanized NSG mice over time treated with ACT subcomponents or combined ACT at equivalent doses (n=6-8 per group). All error bars show S.E.M., statistical tests used are two-way ANOVA (C and I), and t-test comparing combined ACT with a pooled control of all single ACT (D and E). * p<0.05, ** p<0.01, *** p<0.001, **** p<0.0001.

FIG. 2: TRI-IT synergistically eradicates established, poorly immunogenic tumors. (A) experimental overview of in vivo experimental workflow for (B-C), (B) mean fold change of subcutaneous B16F10 melanoma tumor volumes in C57BL/6J mice over time in indicated groups* (n=6-16 per group, pooled from multiple independent experiments), (C) mean fold change of subcutaneous KP lung cancer tumor volumes in C57BL/6J mice over time in indicated groups* (n=5-18 per group, pooled from multiple independent experiments), (D) experimental overview of in vivo experimental workflow for (E-H), (E-F) Kaplan-Meier survival plot of established B16F10 melanoma (E) or KP lung cancer (F) bearing C57BL/6J mice treated with TRI-IT. (G-H) Kaplan-Meier survival plot of previously B16F10 melanoma (G) or KP lung cancer (H) bearing C57BL/6J mice surviving until day 60 after TRI-IT treatment being re-challenged with B16F10 melanoma (G) or KP lung cancer (H). All error bars show S.E.M., statistical tests used are pairwise two-way ANOVA (TRI-IT vs. other) (B and C). *The ACT mono groups in (B) and (C) show reduced tumor growth inhibition compared to FIG. 1C, because ACT was given without lymphodepletion in these experiments, whereas lymphodepletion was included in FIG. 1C.

FIG. 3: TRI-IT orchestrates a broad anti-tumor immune response in poorly immunogenic tumors. (A-B) quantification of anti-tumor antibody response in sera of subcutaneous B16F10 (A) or KP (B) bearing C57BL/6J mice on day 21 (B16F10) or day 24 (KP) in different treatment groups shown as fold change of geometric mean fluorescence intensity measured by FACS compared to tumor-naïve control mice (n=3-14 per group, pooled from multiple independent experiments, see methods for details,*), (C-D) quantification of cellular anti-tumor immunity in splenocytes of B16F10 bearing C57BL/6J mice on day 21 in different treatment groups by (C) cytotoxicity towards B16F10 cells (n=3-8 per group, +) or (D) intracellular IFNγ response measured by FACS upon B16F10 re-stimulation (n=2-5 per group), (E-J) immune cell infiltration into subcutaneous B16F10 bearing C57BL/6J mice on day 21 in different treatment groups measured by FACS (n=4-13 per group, pooled from multiple independent experiments), (K-O) proportion of IFNγ+ cells among indicated subsets of tumor-infiltrating immune cells in subcutaneous B16F10 bearing C57BL/6J mice on day 21 measured by intracellular FACS (n=4-13 per group, pooled from multiple independent experiments), (P) heatmap of mean z-scores of cytokines quantified by multiplex Luminex analysis in the sera of pooled B16F10 or KP bearing C57BL/6J mice sacrificed on day 21 (B16F10) or day 24 (KP) mice treated in different experimental groups with groups of cytokines altered similarly in a treatment group highlighted in green, (Q) Correlation between observed log 2 fold change and log 2 fold change predicted by the final PLR model using cytokines quantified in sera as inputs to predict end of experiment tumor size (see methods for details), (R) coefficients and VIP scores (measure of importance of input variable in model) for all cytokines with a VIP score >1 in the final PLR model, (S) gene set enrichment analysis of day 24 KP tumors for selected gene sets from TRI-IT-treated mice versus pooled mice from all other treatment groups, (T) mean gene expression z-scores of immune checkpoint transcripts quantified in pooled day 21 B16F10 and day 24 KP tumors (n=6-23 per group, pooled from multiple independent experiments), (U) immune cell deconvolution analysis showing mean gene expression z-scores of activated dendritic cell specific transcripts (n=6-23 per group, pooled from multiple independent experiments). All error bars show S.E.M, statistical tests used are t-test (A-C and E-O), Dunn's test (D and U) and one-way ANOVA (T). +, stars indicate significance level of t-test compared to TRI-IT group, * p<0.05, ** p<0.01, *** p<0.001, **** p<0.0001.

FIG. 4: Depletions of CD4+ and CD8+ T-cells, NK-cells, γδ-T-cells and macrophages reduce TRI-IT anti-tumor effect. (A) experimental overview of in vivo experimental workflow, (B) mean fold change of subcutaneous B16F10 melanoma tumor volumes in C57BL/6J mice overtime in indicated depletion groups (n=6-8 per group), (C) quantification of cellular anti-tumor immunity in splenocytes of B16F10 bearing C57BL/6J mice on day 21 in different treatment groups by measuring cytotoxicity towards B16F10 cells (n=3-4 per group, see methods for details), (D) quantification of anti-tumor antibody response in sera of subcutaneous B16F10 bearing C57BL/6J mice on day 21 in different depletion groups shown as fold change of geometric mean fluorescence intensity measured by FACS compared to tumor-naïve control mice (n=2-4 per group, see methods for details), (E-I) immune cell infiltration into subcutaneous B16F10 bearing C57BL/6J mice on day 21 in different depletion groups measured by FACS (n=8-13 per group, pooled from multiple independent experiments). All error bars show S.E.M, statistical tests used are two-tailed, unpaired t tests (A and C-I) and two-way ANOVA (B).

FIG. 5: TRI-IT is effective in autologous humanized patient-derived mouse models of lung cancer. (A) experimental overview of in vivo experimental workflow, (B) FACS plots showing proportion of intracellularly IFNγ+ cells among CD8+ T-cells after re-stimulation of indicated CTLs with either no target cells or indicated PDX cells, (C-D) proportion of intracellularly IFNγ+ cells among CD8+(C) or CD4+(D) T-cells after re-stimulation of indicated CTLs with either no target cells or indicated PDX cells, (E-G) mean fold change of indicated PDX tumor growth in autologously humanized NSG mice over time in indicated treatment groups (n=5-8 per group), (H) mean levels of indicated cytokines quantified by multiplex Luminex analysis in the sera of PDX1.1 bearing autologously humanized NSG mice sacrificed on day 24 treated in indicated experimental groups (n=3 per group). (I) immune cell infiltration on day 24 (PDX1) or day 37 (PDX7) into indicated PDX tumors growing in autologously humanized mice in indicated treatment groups measured by FACS (n=6-8 per group), (J) proportion of IFNγ+ cells among indicated subsets of tumor-infiltrating immune cells on day 24 (PDX1) or day 37 (PDX7) into indicated PDX tumors growing in autologously humanized mice in indicated treatment groups measured by intracellular FACS (n=6-7 per group), (K) comparison of injected versus not-injected tumor growth fold change at the end of the experiment in pooled PDX 1.1, PDX 1.2 or PDX 7 tumors (n=12 per group). All error bars show S.E.M, statistical tests used are two-tailed, unpaired t test for (E-H) and two-way ANOVA with significance of the variable ‘group allocation’ reported (I-J). * p<0.05, ** p<0.01, *** p<0.001.

FIG. 6: TRI-IT shows synergistic treatment effects in an autochthonous genetically engineered lung cancer mouse model. (A) experimental overview of in vivo experimental workflow, (B) representative μCT images of autochthonous KP lung cancers in indicated treatment groups at indicated times after treatment initiation, (C) mean fold change of autochthonous KP lung cancer lesion growth over time in indicated treatment groups (n=8-29 lesions per group), (D-F) indicated measures of tumor burden at end of experiment measured on HE stained coronal cuts through both lungs (n=5-8 mice per group), (G) infiltration of tumors by indicated immune cell subsets at end of experiment measured on IHC of coronal cuts through both lungs (n=4-7 mice per group), (H) immune cell deconvolution analysis showing mean gene expression z-scores of immune cell specific transcripts (n=4-9 mice per group, see methods for details), (I) mean gene expression z-scores of intratumoral cyto- and chemokines (n=4-9 mice per group), (J) mean gene expression z-scores of selected immune checkpoint transcripts (n=4-9 mice per group), (K) mean gene expression z-scores of transcripts representative of a immunogenic cell death signature (n=4-9 mice per group), (L) mean differential gene expression z-score of transcripts representative of a M1/M2 macrophage signature indication M2 to M1 shift (n=4-9 mice per group, see methods for details), (M) heatmap showing most differentially expressed transcripts (aPD1 vs. any TRI-IT) (n=4-9 mice per group), (N) gene set enrichment analysis for selected gene sets from any TRI-IT vs. aPD1 treated autochthonous KP lung tumors. All error bars show S.E.M, statistical tests used are a mixed-effects model (C), t-tests between indicated groups (D-I, L) and two-way ANOVA with significance of the variable ‘group allocation’ reported (J-K). * p<0.05, ** p<0.01, *** p<0.001, **** p<0.0001.

FIG. 7: TRI-IT exhibits no off-target, immune-mediated toxicity. (A) experimental overview of in vivo experimental workflow, (B) mean relative weight change of mice in indicated groups over time (n=4 per group), (C-E) lung (C), liver (D) or spleen (E) weight in percent of body weight at the end of experiment (day 14) in indicated groups (n=4 per group), tu: tumor, (F-G) enumeration (F) of CD3+ T-cells infiltrating the liver per field of view by immunohistochemistry and accompanying representative (G) images (n=3-4 per group), (H-I) enumeration (H) of CD3+ T-cells infiltrating the lung per field of view by immunohistochemistry and accompanying representative (I) images (n=3-4 per group), (J-K) enumeration (J) of CD3+ T-cells infiltrating the intestine per 30 villi by immunohistochemistry and accompanying representative (K) images (n=3-4 per group). All error bars show S.E.M, statistical tests used are two-sided, unpaired t tests (B-E, F, H and J).

FIG. 8: Combined tumor infiltration by T-cells excluding γδ-T-cells, NK-cells and γδ-T-cells predicts superior outcome across human cancer. (A-D) Kaplan-Meier overall survival curves for all cancers combined (A), lung cancer (B), melanoma (C) or sarcoma (D) patients either separating patients by TriScore or below/above median infiltration of NK cells, T-cells excluding γδ-T-cells or γδ-T-cells. Bottom plots show 3-year overall survival deviations of indicated groups from overall survival of the respective whole cohort indicating that TriScore (dark blue) differentiates subgroups of patients with different overall survival superior to below/above median infiltration of NK-cells, T-cells excluding γδ-T-cells or γδ-T-cells, (E) plot showing overall survival difference between lowest vs. highest subgroup of patients for below/above median infiltration of NK-cells, T-cells excluding γδ-T-cells or γδ-T-cells or TriScore in indicated cancer cohorts. All error bars show 95% confidence intervals, statistical test used is two-way ANOVA.

FIG. 9: Combined treatment with lymphokine-activated killer cells (LAKs), cytokine-induced killer cells (CIKs), Vγ9Vδ2-T-cells (γδ-T-cells) and adaptive, tumor-specific T-cells (CTLs) is superior to single agent adoptive cellular therapy (FIG. 8). (A) heatmap showing phenotype of end-of-expansion adoptive cellular therapy subcomponents evaluated by flow cytometry. The proportion of cell expressing each indicated cell surface marker is shown for each indicated ACT subcomponent, (B-C) cytotoxicity of end-of-expansion KP CTL and B16 CTL towards B16F10 (B) and KP (C) targets at indicated effector to target cell ratios (n=2-4 replicated per group), (D-E) heatmap showing proportion of intracellularly IFNγ+ cells among indicated cells (CD3+, CD4+ or CD8+) in end-of-expansion B16 (D) or KP (E) CTLs after re-stimulation with indicated cell lines, (F) Relative change in cytotoxicity of indicated end-of-expansion adoptive cellular therapy subcomponents against indicated cell lines upon co-incubation with indicated blocking antibodies (n=2-4 replicates per group). All error bars show S.E.M, statistical tests used are two-way ANOVA with significance of variable ‘type of CTL’ reported (B-C) and two-sided, unpaired t test of pooled data using both target cell lines (F). * p<0.05, ** p<0.01, *** p<0.001.

FIG. 10: Cytotoxicity of LAKs, CIKs, γδ-T-cells and CTLs can be enhanced by aPD1 antibodies. (A) cytotoxicity enhancement by murine aPD1 antibody co-incubation in cytotoxicity assays using indicated end-of-expansion murine adoptive cellular therapy subcomponents against indicated murine target cell lines at indicated effector to target cell ratios (n=2-6 replicates per group), (B) cytotoxicity enhancement by human aPD1 antibody co-incubation in cytotoxicity assays using indicated end-of-expansion human adoptive cellular therapy subcomponents against indicated human target cell lines at indicated effector to target cell ratios (n=2-5 replicates per group). All error bars show S.E.M, statistical tests used are two-way ANOVA with significance of variable ‘aPD1 vs. isotype control’ reported (A and B). * p<0.05, ** p<0.01, *** p<0.001, **** p<0.0001.

FIG. 11: TRI-IT synergistically eradicates established, poorly immunogenic tumors (FIGS. 9 and 10). (A) spider plot of fold changes of subcutaneous B16F10 melanoma tumor volumes in C57BL/6J mice over time in aPD1 (blue) and TRI-IT (red) group (n=6-16 per group, pooled from multiple independent experiments), (B) spider plot of fold changes of subcutaneous KP lung cancer tumor volumes in C57BL/6J mice over time in aPD1 (blue) and TRI-IT (red) group (n=5-18 per group, pooled from multiple independent experiments), (C-D) comparison of TLR-agonist-injected versus not-TLR-agonist-injected tumor growth fold change at the end of the experiment in B16F10 melanoma (C) or KP lung cancer (D) tumors in indicated groups (n=6-16 per group), (E-F) immune cell infiltration into subcutaneous B16F10 melanoma bearing C57BL/6J mice on day 21 in different treatment groups measured by FACS (n=4-13 per group, pooled from multiple independent experiments), (G-M) immune cell infiltration into subcutaneous KP lung cancer bearing C57BL/6J mice on day 24 in different treatment groups measured by FACS (n=3-12 per group, pooled from multiple independent experiments). All error bars show S.E.M, statistical tests used are two-way ANOVA with significance of variable ‘injected vs. not injected’ reported (C-D) and t-test (E-M).

FIG. 12: TRI-IT is highly effective against human sarcoma. (A) experimental overview of in vivo experimental workflow, (B) heatmap showing proportion of intracellularly IFNγ+ cells among indicated cells (CD3+, CD4+ or CD8+) in end-of-expansion KP sarcoma (B) or MCA/p53 (C) CTLs after re-stimulation with indicated cell lines, (D-E) mean fold change of subcutaneous KP sarcoma (D) or MCA/p53 (E) tumor volumes in 129/SvJ mice over time in indicated groups (n=8 per group), (F) immune cell infiltration into explanted tumors of pooled subcutaneous KP or MCA/p53 sarcoma bearing 129/SvJ mice on day 25 in different treatment groups measured by FACS (n=5-8 per group), (H) proportion of IFNγ+ cells among indicated subsets of tumor-infiltrating immune cells in pooled subcutaneous KP or MCA/p53 sarcoma bearing 129/SvJ mice on day 25 measured by intracellular FACS (n=5-8 per group), CD8+/Treg ratio in pooled subcutaneous KP or MCA/p53 sarcomas or spleens from tumor bearing 129/SvJ mice on day 25 in different treatment groups measured by FACS (n=5-8 per group), (I) volcano plot showing cytokines detected at significantly higher levels in the TRI-IT compared to aPD1 group in the sera of pooled subcutaneous KP or MCA/p53 sarcoma bearing 129/SvJ mice on day 25, (J) coefficients and VIP scores (measure of importance of input variable in model) for all cytokines with a VIP score >1 in the final PLR model using cytokines quantified in sera as inputs to predict end of experiment tumor size (see methods for details). All error bars show S.E.M, statistical tests used are two-way ANOVA (D-E) and two-sided, unpaired t tests (F-G), * p<0.05.

FIG. 13: TRI-IT is effective in allogeneic humanized mouse models of lung and breast cancer—in vitro data. (A) heatmap showing phenotype of end-of-expansion adoptive cellular therapy subcomponents evaluated by flow cytometry. The proportion of cell expressing each indicated cell surface marker is shown for each indicated ACT subcomponent, (B-D) heatmap showing proportion of intracellularly IFNγ+ cells among indicated cells (CD3+, CD4+ or CD8+) in end-of-expansion JimT1 (B), H441 (C) or H1975 (D) CTLs after re-stimulation with indicated cell lines (E) cytotoxicity of indicated end-of-expansion CTLs as effector cells and indicated targeted cells (n=3-12 replicates per group, pooled from multiple independent experiments), (F) relative change in cytotoxicity of indicated end-of-expansion adoptive cellular therapy subcomponents against indicated cell lines upon co-incubation with indicated blocking antibodies (n=2-6 replicates per group), (G) gene scan of T-cell receptor clonality analysis using TCRB multiplex tube B. All error bars show S.E.M, statistical tests used are one-way ANOVA followed by pairwise comparison with multiple testing correction for which significance is reported (E) and two-sided, unpaired t test of pooled data using all 3 target cell lines (F). * p<0.05, *** p<0.001.

FIG. 14: TRI-IT is effective in allogeneic humanized mouse models of lung and breast cancer—in vivo data. (A) experimental overview of in vivo experimental workflow, (B-D) mean fold change of subcutaneous JimT1 breast cancer (B), H441 lung cancer (C) or H1975 lung cancer (D) tumor volumes in humanized NSG mice over time in indicated groups and accompanying waterfall plot showing end of experiment tumor volume fold change (n=6-12 per group, pooled from multiple independent experiments). All error bars show S.E.M.

FIG. 15: TRI-IT is highly effective against human lymphoma. (A) experimental overview of in vivo experimental workflow, (B-C) mean fold change of subcutaneous L428 lymphoma (B) or KMH2 lymphoma (C) tumor volumes in humanized NSG mice over time in indicated groups (n=4-10 per group). All error bars show S.E.M, statistical tests used are two-way ANOVA (B-C).

FIG. 16: Murine immune cell subset depletions are effective and depletions of CD4+ and CD8+ T-cells reduce efficacy of TRI-IT in a humanized mouse model of cancer (FIG. 11). (A) confirmation of successful depletion by measurement of the proportion of immune cell subsets among CD45+ splenocytes in different depletion groups (n=3-4 per group), (K) mean fold change of subcutaneous H1975 lung cancer tumor volumes in humanized NSG mice overtime in indicated depletion groups (n=8 per group). All error bars show S.E.M, statistical tests used are two-tailed, unpaired t tests (A) and two-way ANOVA (B).

### DETAILED DESCRIPTION OF THE INVENTION

The applicants developed, in one embodiment, an antigen-agnostic combination immunotherapy, which is effective in a wide range of poorly immunogenic tumors for which a precise antigenic determinant is not known. In one preferred embodiment of the invention, an antigen-agnostic, tripartite combination immunotherapy was developed, which was named TRI-IT and which is effective in a wide range of poorly immunogenic tumors for which a precise antigenic determinant is not known.

Until now, as detailed above, most commonly used combination immunotherapy approaches that are effective in poorly immunogenic tumors rely on precise knowledge of the antigenic determinants of a tumor and include adoptively transferred cells, vaccinations or antibodies engineered to directly target those.

The antigen-agnostic combination immunotherapy relates, in one embodiment, to a combination therapy with at least two of the following populations of cells (i) to (iv): (i) a population of lymphokine-activated killer cells (LAKs), (ii) a population of cytokine-induced killer cells (CIKs), (iii) a population of γδ-T-cells, (iv) a population of tumor-specific T-cells (CTLs).

As shown in the examples, the administration of at least two of the above different cell populations is useful in a method of preventing or treating cancer and can be applied in a versatile and uncomplicated way. Surprisingly, the combination therapy is effective in poorly immunogenic tumors. Such tumors are difficult to treat.

TRI-IT comprises or consists of 3 elements, combining at least one immune checkpoint targeting agent, such as a checkpoint blockade targeting PD1, local immunotherapy by administration, such as injection into one tumor lesion, of agonists against TLR or other Th-1 adjuvants, and an optimized combined ACT protocol including LAKs, CIKs, γδ-T-cells and tumor-specific CTLs following lymphodepletion, by administering at least one lymphodepleting agent. Moreover, a least one IL-2 family cytokine, such as IL-2 may in addition be administered in the TRI-IT protocol.

The applicants surprisingly found that combination therapy using the above different cell populations have superior effects in the treatment of cancer. Accordingly, the method for use in the treatment of cancers as described herein provides a clinically translatable, universal combination immunotherapy, that is antigen-agnostic and highly efficient in a large variety of cancers.

The term “antigen-agnostic” is understood to mean that the therapy does not require the knowledge and/or identification of tumor antigen(s) of the tumor or cancer to be treated.

Accordingly, in a first aspect of a present invention provided herein is a medicament for use in a method of preventing or treating cancer in a patient, wherein the medicament comprises at least two of the following populations of cells (i) to (iv): (i) a population of lymphokine-activated killer cells (LAKs), (ii) a population of cytokine-induced killer cells (CIKs), (iii) a population of γδ-T-cells, (iv) a population of tumor-specific T-cells (CTLs), wherein the population of cells in (i) to (iv) are derived from autologous cells from said patient or from allogeneic cells from a donor.

According to a preferred embodiment of the present invention, the medicament comprises at least two of the populations of cells (i) to (iv) or comprises the populations of cells of (i), (ii), (iii) and (iv).

Related to the present invention the term “comprise/s/ing”, as used herein, is meant to include or encompass the disclosed features and further features, which are not specifically mentioned. The term “comprise/es/ing” is also meant in the sense of “consist/s/ing of” the indicated features, thus not including further features except the indicated features. Thus, the subject-matter of the present invention may be characterized by additional features in addition to the features as indicated.

The first aspect relates to a medicament for use in a method of preventing and/or treating cancer. A “medicament” as it is used herein can be understood as any substance and/or drug, curing or improving human's or animal's health condition or maintain its health. The terms “treat”, “treating” and “treatment” are to be understood as alleviating or abrogating a disease and/or its attendant symptoms. The term “prevention” or “prevent” are to be understood as treatment that prevents and/or avoids the occurrence of a condition in a subject.

The first aspect of the present invention also refers to specific cell populations.

In one embodiment, a population of lymphokine-activated killer cells (LAKs) is used. “Lymphokine-activated killer cells (LAKs)” are known to the skilled person in art. LAKs are cytolytic lymphocytes with the unique ability to kill NK-resistant fresh human tumor cells in short-term assays. LAKs can kill autologous tumors as well as TNP-modified own and allogeneic tumors with complete cross-reactivity, at the population level as well as at the clonal level. Initial results on the classification of LAKs have so far concluded that LAKs differ from classical NK and T lymphocyte systems on basis of a number of criteria. These include, for example, the surface phenotype, activation conditions and spectrum of target cells differ. LAKs can e.g. be produced by in vitro stimulation of PBMCs with IL-2 and is most efficient under serum-free conditions as described in Mulé, J. J., Shu, S., Schwarz, S. L. & Rosenberg, S. A. Adoptive immunotherapy of established pulmonary metastases with LAK cells and recombinant interleukin-2. Science, 225 (4669), 1487-1489 (1984) (Reference 21). Accordingly, LAKs are obtainable by stimulation of PBMCs in vitro in the presence of IL-2 under serum-free conditions. Further, LAKs are obtainable by stimulation of PBMCs in vitro in the presence of IL-2 under serum-free conditions as described in Mulé, J. J. et al. supra. As described in the examples, human LAKs are obtainable by culturing PBMCs in vitro in cell expansion medium supplemented human IL-2 for 4 to 10 days, such as 7 days, exchanging part of the medium, and increasing culture volume to maintain the initial cell density every 3 days or earlier if needed and harvesting the cells at about days 4 to 10, such as on day 7 of culture.

Furthermore, the first aspect of the present invention refers to “cytokine-induced killer cells (CIKs)”. CIKS are also known to the skilled person in art. CIKs represent a T cell population that combines a T cell and natural killer cell-like phenotype in its terminally differentiated CD3(+)CD56(+) subset and exhibits non-MHC-restricted tumor-killing activity. CIK cells are expandable from PBMCs and mature after the addition of specific cytokines. These include, but are not limited to it, IL-2. CIKs are therefore T cells which are CD3(+)CD56(+) and exhibit non-MHC-restricted tumor-killing activity. Methods for determining tumor-killing activity in vitro and MHC-restriction thereof are known in the art. CIKs are obtainable as follows, as also described in the examples: culturing PBMCs in cell expansion medium supplemented with human IFNγ, exchanged for cell expansion medium supplemented human IL-2, exchanging part of the medium and increasing culture volume to maintain the initial cell density every 3 days or earlier if needed and harvesting CIKs at about 10 to 20 days, such as on day 14 of culture.

The first aspect of the invention also relates to “γδ-T cells” or “γ-δ-T cells”, which are also known to the skilled person in art. γ-δ-T cells are specific T cells that express a unique T-cell receptor (TCR) composed of one γ-chain and one δ-chain. γ-δ-T cells are of low abundance in the body, are found in the gut mucosa, skin, lungs and uterus, and are involved in the initiation and propagation of immune response. γ-δ-T cells are characterized by specific surface markers, carried on the surface of the cell. Surface markers include for example, but are not limited to those: E-Cadherin, CCR5, CCR6, CD3, CD4, CD8, CD27/TNFRSF7, CD27 Ligand/TNFSF7. γ-δ-T cells can be produced by selectively simulating PBMCs with zoledronic acid and specific cytokines. Those can include, but are not limited it, for example II-2. Human γδ-T cells are obtainable by enriching human γδ-T cells from PBMCs. Enriching human γδ-T cells from PBMCs can be performed by culturing PBMCs in vitro in cell expansion medium supplemented with human IL-2 and zoledronic acid, exchanging part of the media, and increasing culture volume appropriately to maintain the initial cell density every 3 days or earlier if needed. Zoledronic acid is not added anymore beyond day 3. γδ-T cells are harvested at days 10 to 20 of culture, such as on day 14 of culture. γδ-T cells can further be identified and/or isolated using FACS analysis as CD45+CD3+FoxP3-γδ-TCR+ cells.

An exemplary γ-δ-T cell is a Vγ9-Vδ2-T cell. Vγ9-Vδ2-T cell were used successfully in the examples, but also other γ-δ-T cells may be used in the invention. For example, γδ-T cells of the Vδ2+ subset may be used. Alternatively, γδ-T cells of the Vδ2-subset may be used.

Furthermore, the first aspect of the present invention relates to “tumor-specific T cells” or “CTLs”. CTLs are also known to the skilled person in the art. CTLs can be produced for example by a co-culture method, relying on autologous tumor organoids and peripheral blood lymphocytes, as described in Dijkstra et al. (Reference 24) herein. Of note, CTLs production as described in Dijkstra et al. (Reference 24) does not need knowledge of tumor-associated antigen. Specifically Human CTLs are obtainable and enriched as described in the example. First, target tumor cells are pretreated with Mitomycin C (such as about 50 or 100 μg/ml) and human IFNγ in cell expansion medium for about 24 h in a regular cell culture incubator (37° C., 5% CO₂) and then washed. The next day, PBMCs are co-cultured with the target tumor cells in cell expansion medium supplemented with human IL-2, 10 ng/ml, human IL-7, anti-human, anti-CD28 and anti-human anti-PD1-antibody. Target tumor cells were added at a 10:1 ratio (PBMCs to tumor cells). Part of the media is exchanged, and culture volume appropriately increased to maintain the initial cell density every 3 days or earlier if needed. With each media exchange, media is only supplemented with recombinant human IL-2 and anti-human anti-PD1-antibody. CTLs are harvested at about 10 to 20 days, such as on day 14 of culture.

Related to the first aspect of the invention, reference is made to a population of cells.

A “population” as it is used herein is to be understood as means a totality of cells which may be of different sizes and which may comprise different cells. A “population” of cells comprises 2, 3, 4, 5, 6, 7, 8, 9, 10 or more cells, such as 10² or more, 10³, or more, 10⁴ or more, 10⁵ or more, 10⁶ or more, 10⁷ or more, 10⁸ or more, or 10⁹ or more, such as up to about 10¹⁰ cells.

The population of cells may be only comprise the indicated cell type, such as a LAKs in case of a population of lymphokine-activated killer cells (LAKs), CIKs in case of a population of cytokine-induced killer cells (CIKs), γδ-T-cells in case of a population of γδ-T-cells, or CTLs in case of a population of tumor-specific T-cells (CTLs), or may comprise other cells, such as for 90%, 80%, 70%, 60%, 50%, 40%, 30%, 20%, 10%, 9%, 8%, 7%, 6%, 5%, 4%, 3%, 2%, 1% or 0.1% of other cells. A population of cells according to the invention does not relate to a naturally occurring population of cells, such as blood or a blood sample retrieved from a patient. The “population of X cells”, wherein X refers to a specific cell type, such as LAK, CIK, CTL or γδ-T-cells, relates to a population of X cells, wherein the X cells were generated in vitro or ex vivo, and/or are enriched from a naturally occurring tissue or fluid sample from a patient. For example, the X cells may be enriched 2-fold, 5-fold, 10-fold, 50-fold, 100-fold, 1000-fold or more.

In the first aspect, reference is also made do autologous and allogeneic cells. These terms are well known to a skilled person in the art. As it is used herein, “autologous cells” is to be understood as cells, which are obtained from the same individual that is to be treated with those cells. “Allogeneic cells” are cells from a different donor/individual.

According to one embodiment of the invention, the populations of cells are derived from autologous cells from said patient or from allogeneic cells from a donor.

“Derived from” is understood as that the populations of cells are obtained by culturing and/or enriching the cells from autologous cells from said patient or from allogeneic cells from a donor. Suitable methods for culturing and/or enriching are described above and in the examples for each of the cell types CIKs, LAKs, γδ-T-cells and CTLs.

In the examples, human PBMCs samples were used as starting material for generating each of the populations of CIKs, LAKs, γδ-T-cells and CTLs, respectively. The populations of CIKs, LAKs, γδ-T-cells and CTLs, respectively are derived from the PBMC sample.

The allogeneic cells from a donor is preferably obtained from the same species. Accordingly, in an embodiment, the patient is a human, and the donor is a human.

The donor is in one embodiment a healthy individual and/or an individual which does not suffer from cancer. In another embodiment, the donor does not exhibit HLA mismatches as compared to the patient. Alternatively, the donor exhibits HLA 6 or less, 5 or less, 4 or less, 3 or less, 2 or less or 1 or less mismatches as compared to the patient.

In one embodiment of the present invention, the medicament comprises at least two of the populations of cells (i) to (iv). In one embodiment, the medicament comprises the populations of cells (i) and (ii). In another embodiment, the medicament comprises the populations of cells (i) and (iii). In one embodiment, the medicament comprises the populations of cells (i) and (iv). In one embodiment, the medicament comprises the populations of cells (ii) and (iii). In one embodiment, the medicament comprises the populations of cells (ii) and (iv). In one embodiment, the medicament comprises the populations of cells (iii) and (iv).

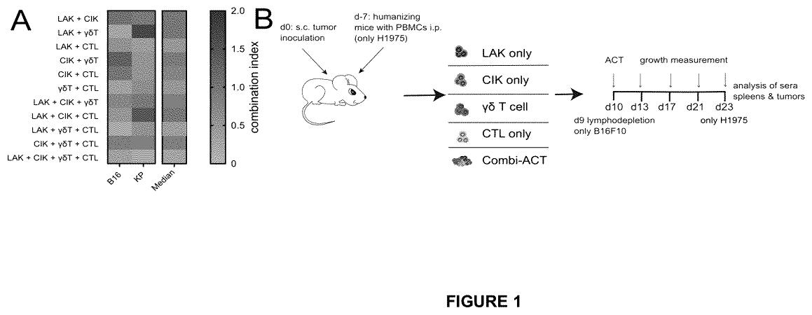

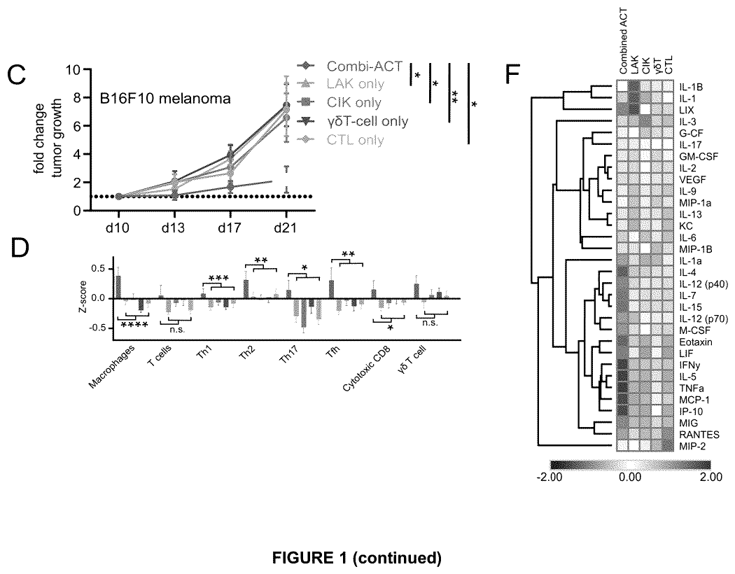

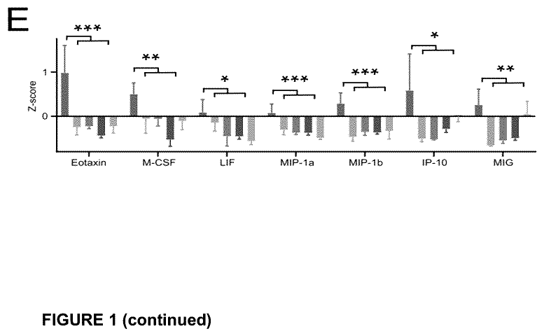

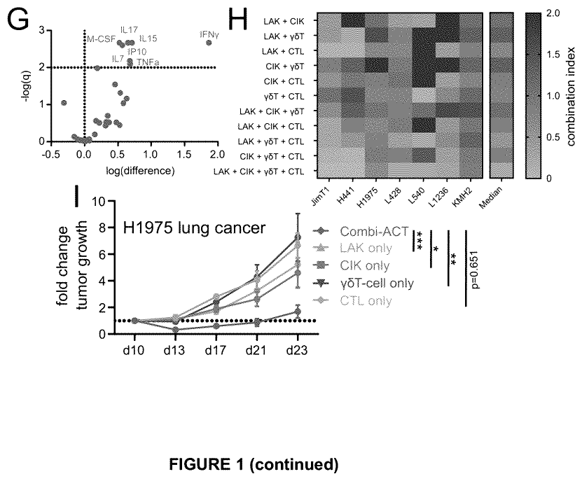

As can be seen in the examples, it was found that various combinations of two of the populations of cells (i) to (iv) exhibits synergistic anti-tumor activity in Example 1 on combined adoptive cellular therapy (see also FIG. 1a).

In one embodiment of the present invention, the medicament comprises at least three of the populations of cells (i) to (iv) or comprises the populations of cells, (i), (ii), (iii), and (iv). In one embodiment, the medicament comprises the populations of cells (i), (ii), and (iii). In one embodiment, the medicament comprises the populations of cells (i), (ii), and (iv). In one embodiment, the medicament comprises the populations of cells (ii), (iii), and (iv). In one embodiment, the medicament comprises the populations of cells (i), (iii), and (iv).

As can be seen in the examples, it was found that various combinations of three of the populations of cells (i) to (iv) exhibit an increasingly synergistic anti-tumor activity in Example 1 on combined adoptive cellular therapy in model systems for poorly immunogenic tumors (see also FIG. 1).

As summarized in Example 1, dual and triple sub-combinations of both innate-acting (e.g. LAKs or CIKs) and tumor-specific effector cells (e.g. CTLs) were more synergistic than innate-only combinations (FIG. 1A).

In still another embodiment, the medicament comprises the populations of cells (i), (ii), (iii), and (iv).

In example 1, the synergy of the quadruple combination of LAKs, CIKs, γδ-T-cells and CTLs (referred to in the examples as “Combined ACT”) and each sub-component at the same cell dose was tested in vivo—i.e., either 1×10⁷ cells of a subcomponent or 2.5×10⁶ cells of each subcomponent for a total of also 1×10⁷ cells—in the B16F10 model (see also FIG. 1B). It was surprisingly found that the quadruple combination of cell populations, i.e. “Combined ACT”, had a stronger inhibitory effect on tumor growth than each ACT subcomponent (FIG. 1C).

In another embodiment of the present invention, (a) the autologous cells from the patient or the allogeneic cells from a donor are cells from a blood sample, preferably a PBMC sample.

Various samples can be used as autologous cells from said patients or as allogeneic cells from a donor. For example, a tissue sample and/or fluidic sample may be used, such as a blood sample, cerebrospinal sample, urine sample, organ tissue sample, mucosal sample or saliva sample. The use of a blood sample is preferred as sufficient amount of immune cells can be retrieved easily from a patient, e.g. a human. As shown in the examples, a PBMC sample was used successfully for generating and/or enriching the populations of cell(s).

In another embodiment of the present invention, the population of tumor-specific T-cells (CTLs) can be enriched and/or generated by cultivation in vitro. As described above, the CTLs can for example be generated by co-cultivating methods, which are known to the skilled person in the art. In another embodiment of the present invention, the population of tumor-specific T-cells is enriched from the autologous cells or from allogeneic cells by co-cultivating the autologous cells or the allogeneic cells with tumor cells from said patient.

All above-mentioned methods for enriching and/or generating cell populations can be performed alone or in combination, and/or in parallel or in sequence, or at the same time point or temporally separate.

The populations of cells are typically in a composition, such as a suspension or cell pellet, which is optionally frozen or freeze-dried. The composition may optionally comprise one or more further agents or excipients, which are preferably pharmaceutically acceptable. For example, the composition may comprise one or more buffering agents, salts, such as PBS and stabilizing excipients. For example, the cell populations may be present as suspension in an aqueous solution, such as an aqueous saline solution.

The two or more populations of cells may be administered as a single composition, or as two or more separate compositions. In one embodiment, the two or more populations of cells are administered as a single composition to the patient. In the examples, the two or more populations of cells, each as suspension, were mixed prior to the administration in vivo. In this embodiment, the populations of cells are maintained in separate containers and are mixed prior to administration or are maintained as single composition for administration. The container may be, for example, an ampoule, vessel, or syringe.

In such embodiment, the two or more populations of cells are administered to the patient at the same time point.

Alternatively, it is possible that each population of cell is administered spatially separate. In such embodiment, at least two of the populations of cells are administered spatially separately. In such embodiment, the populations of cells are maintained in separate containers.

In such embodiment, the two or more populations of cells may be administered to the patient simultaneously or at different time points.

In one embodiment, the populations of cells (i) to (iv) can be administered to said patient in a single composition and simultaneously.

Alternatively, the populations of cells (i) to (iv) are administered to said patient spatially separate and within 24 hours. In one embodiment, the populations of cells (i) to (iv) can be administered to said patient within about 12 to 24 hours. Typically, the two or more populations of cells are administered to the patient within 24 hours, such as within 20, 15, 12 10, 5, 4, 3, 2, or 1 hour(s).

The amounts of cells to be administered may vary. Typically, for each population of cell(s), between about 10⁶ to about 10¹⁰ cells of the two or more populations of cells are administered to the patient. In one embodiment, between about 5×10⁷ to about 5×10⁹ cells, or about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹, about 9×10⁹ or about 10¹⁰ cells or any subrange thereof may be administered.

In a further embodiment, the method further comprises administering to the patient at least one Th-1 adjuvant.

An “adjuvant” refers to an agent modifying the effect of other agents while having few if any direct effects when given by itself, but may increase the efficacy or potency of other drugs when administered simultaneously. In immunology and according to the present invention, an “adjuvant” is an agent, which, while not having any specific antigenic effect in itself, may stimulate the immune system, enhancing the response to a vaccine.

In another preferred embodiment of the present invention, the Th1 adjuvant is a danger signal.

In one embodiment, the at least one Th-1 adjuvant is administered once or repeatedly to the patient. For example, the at least one Th-1 adjuvant is administered once, twice, 3, 4, 5, 6, or more times. For example, 1, 2, 3, 4 or 5 Th-1 adjuvant(s) is/are administered. The first administration of the at least one Th-1 adjuvant may be performed within day 2 prior to the administration of the population(s) of cells and day 3 after the administration of the population(s) of cells, preferably within the day of the administration of the population(s) of cells and day 3 after the administration of the population(s) of cells.

In another embodiment, the at least one Th-1 adjuvant is administered systemically or locally. Preferably, the at least one Th-1 adjuvant is administered by inhalation, subcutaneously, by injection, intravenously or intratumorally.

The at least one Th1-adjuvants may be comprised in a solution, such as an aqueous solution, or dried or freeze-dried, and optionally further containing pharmaceutically acceptable excipients.

In one embodiment of the present invention, the at least one Th-1 adjuvant is an adjuvant, which is a danger signal. Such Th-1 adjuvants which are danger signal are well known in the art. For example TLR agonists may be used, as in the Examples.

The Pattern Recognition Receptors (PRR) are known to be “danger” signal receptors in innate cells and APC, which sense Pathogen-Associated Molecular Patterns (PAMPs), which are structural components specific to microbes. Known classes of PRR are toll-like receptors (TLR), retinoic acid-inducible gene I (RIG-1), and Melanoma Differentiation-Associated protein 5 (MDA5), NOD-like receptors, and C type lectins. Within these classes, PRR which trigger a Th1/Tc1 immune reaction are well-known to the person skilled in the art. A “Th1 adjuvant which is a danger signal” induces and supports a Th1/Tc1 cytotoxic response by activating one of the PRR responsible for triggering a Th1/Tc1 immune response, including secretion of IFN-γ by T cells and induction of T cell cytotoxicity. Various PRR trigger a rather uniform Th1/Tc1 immune response directed at elimination of cells harboring intracellular pathogens.

A “Th1 adjuvant which is a danger signal” is understood to be selected from a Pathogen-Associated Molecular Pattern (PAMP, e.g. LPS), a derivative of a PAMP (e.g. MPL and GLA as derivatives of LPS), or an artificial compound, such as a peptide, protein, lipid, lipoprotein, carbohydrate, nucleic acid or small molecule, mimicking a PAMP and activating one or several Pattern Recognition Receptors (PRR). Examples of such artificial compounds are poly (I:C), mimicking double-stranded viral RNA and activating TLR3, or CpG mimicking unmethylated bacterial DNA and activating TLR7/8), or small molecules agonists.

In one embodiment, the at least one Th-1 adjuvant is an adjuvant, which is a danger signal which is selected from a STING agonist, a RIG-1 agonist, an MD5 agonist, a TLR2 agonist, a TLR3 agonist, a TLR4 agonist, a TLR5 agonist, a TLR8 agonist, a TLR9 agonist, or a combination thereof.

In one embodiment, the at least one Th-1 adjuvant is selected from a STING agonist, preferably a STING activating cyclic dinucleotide or a small molecule, a RIG-1 agonist, preferably selected from poly (I:C), polylCLC, poly I:C12U, polyI:C12C, an RNA-based RIG-1 agonist, and a small molecule RIG-1 agonist, an MD5 agonist preferably selected from poly (I:C), polylCLC, poly I:C12U, polyI:C12C, an RNA-based MD5 agonist, and a small molecule MD5 agonist, a TLR2 agonist, preferably selected from Pam3CSK4, Pam2Cys, Pam3Cys, lipoproteins, porins, toxins, and small molecule TLR2 agonists, a TLR3 agonist, preferably selected from poly (I:C), polylCLC, ARNAX, poly I:C12U, polyI:C12C, further dsRNA mimicks, dsRNA and small molecule TLR3 agonists, a TLR4 agonist, preferably selected from MPLA and GLA, and formulations thereof, small molecule TLR4 agonists, a TLR4 agonist, preferably selected from Flagellin and derivatives thereof and small molecule TLR5 agonists, a TLR7 agonist, preferably selected from an imidazoquinolamine, a guanosine analogue, ssRNA, and small molecule TLR7 agonists, a TLR8 agonist, preferably selected from an imidazoquinolamine, a guanosine analogue, ssRNA, and small molecule TLR8 agonists, a TLR9 agonist, preferably selected from a CpG poly- or oligodeoxynucleotide or a variant thereof, and a short DNA oligonucleotide.

In the example, TLR3 agonists, TLR7 agonists and TLR9 agonists were used. Specifically, the TLR agonists used in the Examples were Poly I:C (1.25 mg/kg/BW), Gardiquimod (1.25 mg/kg/BW) and ODN-2395 (5′-tcgtcgttttcggcgcgcgccg-3′ with phosphorothioate bonds, 1.25 mg/kg/BW). These were given twice weekly diluted in PBS intratumorally, or peritumorally if tumors were too small to be directly injected, per dose. In one experiment TLR agonists were given inhaled at similar concentrations after isoflurane anesthesia.

Accordingly, the Th-1 agonists may be typically administered at concentrations of about 0.01 mg/kg BW to about 100 mg/kg BW, such as 0.05, 0.1, 1, 5, 10, 50, or 90 100 mg/kg BW, or any subrange thereof.

In another embodiment, method further comprises administering to the patient at least one IL-2 family cytokine. The term “interleukin-2 family cytokine” or “IL-2 family cytokine” as used herein, refers to any native IL-2, IL-4, IL-7, IL-9, IL-15, or IL-21 from any vertebrate source, including mammals such as primates (e.g. humans) and rodents (e.g., mice and rats), unless otherwise indicated. The term encompasses naturally occurring variants of IL-2 family cytokines, e.g. splice variants or allelic variants. In one embodiment, the native IL-2, native IL-4, native IL-7, native IL-9, native IL-15, or native IL-21 is native human IL-2, native human IL-4, native human IL-7, native human IL-9, native human IL-15, or native human IL-21. In one embodiment, native IL-2, IL-4, IL-7, IL-9, IL-15, or IL-21 is mature IL-2, IL-4, IL-7, IL-9, IL-15, or IL-21, i.e. IL-2, IL-4, IL-7, IL-9, IL-15, or IL-21 lacking the signal peptide. In one embodiment, the at least one IL-2 family cytokine is selected from mature human IL-2 or a fusion protein thereof. In another embodiment, the at least one IL-2 family cytokine is selected from mature human IL-4 or a fusion protein thereof. In another embodiment, the at least one IL-2 family cytokine is selected from mature human IL-7 or a fusion protein thereof. In another embodiment, the at least one IL-2 family cytokine is selected from mature human IL-9 or a fusion protein thereof. In another embodiment, the at least one IL-2 family cytokine is selected from mature human IL-15 or a fusion protein thereof. In another embodiment, the at least one IL-2 family cytokine is selected from mature human IL-21 or a fusion protein thereof.

In another embodiment of the present invention, the at least one IL-2 family cytokine is administered to the patient on the same day as the populations of cells of (i) to (iv), and/or after said day. In another embodiment, the at least one IL-2 family cytokine is administered to the patient two, three or four days after said day. In one preferred embodiment, the at least one IL-2 family cytokine is administered to the patient on the same day as the populations of cells of (i) to (iv), and/or on the day after said day.

In one embodiment, the at least one IL-2 family cytokine is administered repeatedly. For example, the at least one IL-2 family cytokine is administered 1, 2, 3, 4, 5, 6, 7, 8, 9, 10 or more times, or continuously. For example, administration may be continued or repeated until unacceptable toxicity is observed.

For example, the at least one IL-2 family cytokine is administered twice, 3 times, 4 times, or up to 10 times, such as twice daily, daily, every two days or once or twice a week. Alternatively, the at least one IL-2 family cytokine is administered continuously, such as by infusion. In another embodiment, the at least one IL-2 family cytokine is administered once.

The IL-2 family cytokine may be comprised in a solution, such as an aqueous solution, or dried or freeze-dried, and optionally further containing pharmaceutically acceptable excipients. For example, Aldesleukin, which is mature human IL-2 (rh-IL-2), may be used. More specifically, Aldesleukin does not have an N-terminal alanine, the amino acid cysteine at position 125 is substituted by serine and the molecule is not glycosylated. Aldesleukin is commercially available and as a powder for generating an aqueous solution for injection.

Typically, the at least one IL-2 family cytokine is administered at a dose of from 1 to 10000 μg/kg BW IL-2 family cytokine. For example, the IL-2 family cytokine is administered at a dose of from 1, 10, 20, 30, 40 or 50 to 100, 200, 300, 400, 800, 1000, 5000 or 100000 IL-2 family cytokine μg//kg BW persingle administration, such as per daily administration.

In a further embodiment, the method further comprises administering to a patient at least one immune checkpoint targeting agent. In certain embodiments, the checkpoint targeting agent is selected from the group consisting of an antagonist anti-PD-1 antibody, an antagonist anti-PD-L1 antibody, an antagonist anti-PD-L2 antibody, an antagonist anti-CTLA-4 antibody, an antagonist anti-TIM-3 antibody, an antagonist anti-LAG-3 antibody, an antagonist anti-CEACAM1 antibody, an antagonist anti-TIGIT antibody, an agonist anti-CD137 antibody, an agonist anti-ICOS antibody, an agonist anti-GITR antibody, and an agonist anti-OX40 antibody or an inhibitor of indoleamine-2,3-dioxygenase (IDO).

In one preferred embodiment, an antagonistic agent, such as an antagonistic antibody, directed against an inhibitory checkpoint target may be used as immune checkpoint targeting agent. Suitable inhibitory checkpoint targets include for example PD-1, CTLA-2, LAG-3, TIM-3, PD-L1, PD-L2, TIGIT and CEACAM.

In another preferred embodiment, an agonistic agent, such as an agonistic antibody, directed against a stimulatory checkpoint target may be used immune checkpoint targeting agent. Suitable stimulatory checkpoint targets include for example CD137, ICOS, GITR and OX40.

In certain preferred embodiments, the immune checkpoint targeting agent is an immune checkpoint inhibitor.

In the examples, antagonist anti-PD1 antibody Nivolumab was successfully used. The use of an antagonist anti-PD-1 antibody is therefore preferred.

Further suitable immune checkpoint targeting agent are known in the art. For example, a suitable anti-CTLA-4 antibody is for example ipilimumab or tremelimumab, a suitable anti-CD137 antibody is for example urelumab or utomilumab, and a suitable anti-OX40 antibody is for example pogalizumab or tavolixizumab.

The term “PD-1” refers to the protein programmed cell death protein 1. PD-1 nucleotide and amino acid sequences are well known in the art. An exemplary human PD-1 amino acid sequence is set forth in GenBank deposit GI: 167857792.

Further, inhibitors of indoleamine-2,3-dioxygenase (IDO) are known in the art. Suitable IDO inhibitors which can be used include epacadostat, F001287 (Flexus Biosciences/Bristol-Myers Squibb), indoximod (NewLink Genetics), and NLG919 (NewLink Genetics).

In one preferred embodiment, the immune checkpoint targeting agent is an antibody.

In one preferred embodiment, the immune checkpoint targeting agent is an antibody.

In another preferred embodiment, the immune checkpoint targeting agent is an antagonistic antibody directed against a target selected from PD-1, CTLA-2, LAG-3, TIM-3, PD-L1, PD-L2, TIGIT and CEACAM.

In another preferred embodiment, the immune checkpoint targeting agent is an agonistic antibody directed against a target selected from CD137, ICOS, GITR and OX40.

As used herein, the terms “antibody” and “antibodies” include full length antibodies, antigen-binding fragments of full length antibodies, and molecules comprising antibody CDRs, VH regions or VL regions. Examples of antibodies include monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In a specific embodiment, the antibody is a humanized or chimeric antibody. In another specific embodiment, the antibody is a human monoclonal antibody.

The amount of an antibody or composition which will be effective in the treatment and/or prevention of a condition will depend on the nature of the disease, and can be determined by standard clinical techniques. The precise dose to be employed in a composition will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight and health), whether the patient is human or an animal, other medications administered, or whether treatment is prophylactic or therapeutic. Usually, the patient is a human but non-human mammals including transgenic mammals can also be treated. Treatment dosages are optimally titrated to optimize safety and efficacy.

In one embodiment, the at least one immune checkpoint targeting agent is administered to the patient on the same day as the populations of cells of (i) to (iv), and/or after said day. In another embodiment, the least one immune checkpoint targeting agent is administered to the patient two, three or four days after said day. In one preferred embodiment, the at least one immune checkpoint targeting agent is administered to the patient on the same day as the populations of cells of (i) to (iv), and/or on the day after said day.

In another embodiment, the at least one immune checkpoint targeting agent is administered to the patient repeatedly. In another embodiment, the at least one immune checkpoint targeting agent is administered to the patient once. For example, the at least one immune checkpoint targeting agent is administered 1, 2, 3, 4, 5, 6, 7, 8, 9, 10 or more times, or continuously, for example, administration is continued or repeated until unacceptable toxicity is observed.

For example, the at least one immune checkpoint targeting agent is administered twice, 3 times, 4 times, or up to 10 times, such as twice daily, daily, every two days or once or twice a week. Alternatively, the at least one immune checkpoint targeting agent is administered continuously, such as by infusion.

In a further embodiment, the method further comprises administering to a patient at least one lymphodepleting agent. Lymphodepletion is understood as the destruction of lymphocytes and T cells. Lymphodepletion is typically performed prior to an immunotherapy, such as CAR T cell adoptive cell therapy. It is commonly used in the prior art and well known the skilled person. In this context, “lymphodepleting agents” are therefore agents that induce the state of lymphodepletion.

In one embodiment, the at least one lymphodepleting agent is administered at least one day prior to the day on which the populations of cells of (i) to (iv) are administered. In another embodiment, the at least one lymphodepleting agent is administered at least two days prior to the day on which the populations of cells of (i) to (iv) are administered. In one embodiment, the at least one lymphodepleting agent is administered about 1 to 7 days, such as 1, 2, 3, 4, 5, 6, and/or 7 days prior to the day on which the populations of cells of (i) to (iv) are administered. In another embodiment, the at least one lymphodepleting agent is selected from the group consisting of cyclophosphamide, fludarabine or a combination thereof. In one embodiment, both cyclophosphamide and fludarabine are administered. Such combination of cyclophosphamide and fludarabine was used successfully in the Examples. In one embodiment, cyclophosphamide and fludarabine are administered spatially separate or together, and/or are administered at the same time point and/or temporally separate. Typical doses of cyclophosphamide and fludarabine are well known in the art. For example, doses of cyclophosphamide of about 1 to 500 mg/kg BW/d may be administered, such as 10, 20, 30, 40, 50, 60, 70, 80, 90 or 100 mg/kg BW/d. for example, cyclophosphamide may be administered once, twice, 3 times or more. For example, doses of fludarabine of about 0.1 to 100 mg/kg BW/d may be administered, such as 4, 10, 20, 25, 30, 40, 50 or 100 mg/kg BW/d. For example, fludarabine may be administered once, twice, 3 times, 4 times, 5 times or more. Typical regimens for lymphodepletion known in the art involve administration of cyclophosphamide, at about day −4, and administration of fludarabine at days −3, −2 and/or −1 of the administration of the population(s) of cells. In another embodiment, the lymphodepleting agent is radiation therapy.

All of the above-mentioned further administered components can be administered spatially together, in several combinations, or each spatially separate.

Moreover, in one embodiment, the method of the invention comprises administering to the patient populations of cells (i) and (ii) and at least one Th-1 adjuvant. In another embodiment, the method comprises administering to the patient populations of cells (i) and (ii) and at least one Th-1 adjuvant and/or at least one IL-2 family cytokine. In another embodiment, the method comprises administering to the patient populations of cells (i) and (ii) and at least one Th-1 adjuvant and/or at least one IL-2 family cytokine and/or at least one immune checkpoint targeting agent. In another embodiment, the method comprises administering to the patient populations of cells (i) and (ii) and at least one Th-1 adjuvant and/or at least one IL-2 family cytokine and/or at least one immune checkpoint targeting agent and/or at least one lymphodepleting agent.

Moreover, in one embodiment, the method comprises administering to the patient populations of cells (i) and (iii) and at least one Th-1 adjuvant. In another embodiment, the method comprises administering to the patient populations of cells (i) and (iii) and at least one Th-1 adjuvant and/or at least one IL-2 family cytokine. In another embodiment, the method comprises administering to the patient populations of cells (i) and (iii) and at least one Th-1 adjuvant and/or at least one IL-2 family cytokine and/or at least one immune checkpoint targeting agent. In another embodiment, the method comprises administering to the patient populations of cells (i) and (iii) and at least one Th-1 adjuvant and/or at least one IL-2 family cytokine and/or at least one immune checkpoint targeting agent and/or at least one lymphodepleting agent.

Moreover, in one embodiment, the method comprises administering to the patient populations of cells (i) and (iv) and at least one Th-1 adjuvant. In another embodiment, the method comprises administering to the patient populations of cells (i) and (iv) and at least one Th-1 adjuvant and/or at least one IL-2 family cytokine. In another embodiment, the method comprises administering to the patient populations of cells (i) and (iv) and at least one Th-1 adjuvant and/or at least one IL-2 family cytokine and/or at least one immune checkpoint targeting agent. In another embodiment, the method comprises administering to the patient populations of cells (i) and (iv) and at least one Th-1 adjuvant and/or at least one IL-2 family cytokine and/or at least one immune checkpoint targeting agent and/or at least one lymphodepleting agent.

Moreover, in one embodiment, the method comprises administering to the patient populations of cells (ii) and (iv) and at least one Th-1 adjuvant. In another embodiment, the method comprises administering to the patient populations of cells (ii) and (iv) and at least one Th-1 adjuvant and/or at least one IL-2 family cytokine. In another embodiment, the method comprises administering to the patient populations of cells (ii) and (iv) and at least one Th-1 adjuvant and/or at least one IL-2 family cytokine and/or at least one immune checkpoint targeting agent. In another embodiment, the method comprises administering to the patient populations of cells (ii) and (iv) and at least one Th-1 adjuvant and/or at least one IL-2 family cytokine and/or at least one immune checkpoint targeting agent and/or at least one lymphodepleting agent.

Moreover, in one embodiment, the method comprises administering to the patient populations of cells (iii) and (iv) and at least one Th-1 adjuvant. In another embodiment, the method comprises administering to the patient populations of cells (iii) and (iv) and at least one Th-1 adjuvant and/or at least one IL-2 family cytokine. In another embodiment, the method comprises administering to the patient populations of cells (iii) and (iv) and at least one Th-1 adjuvant and/or at least one IL-2 family cytokine and/or at least one immune checkpoint targeting agent. In another embodiment, the method comprises administering to the patient populations of cells (iii) and (iv) and at least one Th-1 adjuvant and/or at least one IL-2 family cytokine and/or at least one immune checkpoint targeting agent and/or at least one lymphodepleting agent.

Further, in one embodiment, the method comprises administering to the patient populations of cells (i) and (ii) and (iii) and at least one Th-1 adjuvant. In another embodiment, the method comprises administering to the patient populations of cells (i) and (ii) and (iii) and at least one Th-1 adjuvant and/or at least one IL-2 family cytokine. In another embodiment, the method comprises administering to the patient populations of cells (i) and (ii) and (iii) and at least one Th-1 adjuvant and/or at least one IL-2 family cytokine and/or at least one immune checkpoint targeting agent. In another embodiment, the method comprises administering to the patient populations of cells (i) and (ii) and (iii) and at least one Th-1 adjuvant and/or at least one IL-2 family cytokine and/or at least one immune checkpoint targeting agent and/or at least one lymphodepleting agent.

Further, in one embodiment, the method comprises administering to the patient populations of cells (i) and (ii) and (iv) and at least one Th-1 adjuvant. In another embodiment, the method comprises administering to the patient populations of cells (i) and (ii) and (vi) and at least one Th-1 adjuvant and/or at least one IL-2 family cytokine. In another embodiment, the method comprises administering to the patient populations of cells (i) and (ii) and (iv) and at least one Th-1 adjuvant and/or at least one IL-2 family cytokine and/or at least one immune checkpoint targeting agent. In another embodiment, the method comprises administering to the patient populations of cells (i) and (ii) and (iv) and at least one Th-1 adjuvant and/or at least one IL-2 family cytokine and/or at least one immune checkpoint targeting agent and/or at least one lymphodepleting agent.

Further, in one embodiment, the method comprises administering to the patient populations of cells (i) and (iii) and (iv) and at least one Th-1 adjuvant. In another embodiment, the method comprises administering to the patient populations of cells (i) and (iii) and (vi) and at least one Th-1 adjuvant and/or at least one IL-2 family cytokine. In another embodiment, the method comprises administering to the patient populations of cells (i) and (iii) and (iv) and at least one Th-1 adjuvant and/or at least one IL-2 family cytokine and/or at least one immune checkpoint targeting agent. In another embodiment, the method comprises administering to the patient populations of cells (i) and (iii) and (iv) and at least one Th-1 adjuvant and/or at least one IL-2 family cytokine and/or at least one immune checkpoint targeting agent and/or at least one lymphodepleting agent.

Further, in one embodiment, the method comprises administering to the patient populations of cells (ii) and (iii) and (iv) and at least one Th-1 adjuvant. In another embodiment, the method comprises administering to the patient populations of cells (ii) and (iii) and (iv) and at least one Th-1 adjuvant and/or at least one IL-2 family cytokine. In another embodiment, the method comprises administering to the patient populations of cells (ii) and (iii) and (iv) and at least one Th-1 adjuvant and/or at least one IL-2 family cytokine and/or at least one immune checkpoint targeting agent. In another embodiment, the method comprises administering to the patient populations of cells (ii) and (iii) and (iv) and at least one Th-1 adjuvant and/or at least one IL-2 family cytokine and/or at least one immune checkpoint targeting agent and/or at least one lymphodepleting agent.

Finally, in one embodiment, the method comprises administering to the patient populations of cells (i) and (ii) and (iii) and (iv) and at least one Th-1 adjuvant. In another embodiment, the method comprises administering to the patient populations of cells (i) and (ii) and (iii) and (iv) and at least one Th-1 adjuvant and/or at least one IL-2 family cytokine. In another embodiment, the method comprises administering to the patient populations of cells (i) and (ii) and (iii) and (iv) and at least one Th-1 adjuvant and/or at least one IL-2 family cytokine and/or at least one immune checkpoint targeting agent. In another embodiment, the method comprises administering to the patient populations of cells (i) and (ii) and (iii) and (iv) and at least one Th-1 adjuvant and/or at least one IL-2 family cytokine and/or at least one immune checkpoint targeting agent and/or at least one lymphodepleting agent.

The invention further relates, in another aspect, to a medicament for use in a method of preventing or treating cancer in a patient, wherein the medicament comprises at least one of the following populations of cells (i) to (iv), wherein the cells in (i) to (iv) are derived from autologous cells from said patients or from allogeneic cells from a donor, and wherein the method comprises administering to the patient: the at least one of the populations of cells of (i) to (iv), (v) the at least one Th-1 adjuvant and/or (vi) the at least one IL-2 family cytokine, (vii) and/or the at least one immune checkpoint targeting agent, and/or (viii) the at least one lymphodepleting agent.

The invention further relates, in another aspect, to a medicament for use in a method of preventing or treating cancer in a patient, wherein the medicament comprises at least one of the following populations of cells (i) to (iv), wherein the cells in (i) to (iv) are derived from autologous cells from said patients or from allogeneic cells from a donor, and wherein the method comprises administering to the patient: the at least one of the populations of cells of (i) to (iv), (v) the at least one Th-1 adjuvant and (vi) the at least one IL-2 family cytokine, (vii) and the at least one immune checkpoint targeting agent, and, optionally (viii) the at least one lymphodepleting agent.

The invention further relates, in a second aspect, to a medicament for use in a method of preventing or treating cancer in a patient, wherein the medicament comprises at least one of the following populations of cells (i) to (iv), wherein the cells in (i) to (iv) are derived from autologous cells from said patients or from allogeneic cells from a donor, and wherein the method comprises administering to the patient: the at least one of the populations of cells of (i) to (iv), (v) the at least one Th-1 adjuvant and (vi) the at least one IL-2 family cytokine, (vii) and the at least one immune checkpoint targeting agent, and (viii) the at least one lymphodepleting agent.

Accordingly, in one embodiment of the present invention, the method comprises administering to the patient the population of cells (i) and at least one Th-1 adjuvant and at least one IL-2 family cytokine and at least one immune checkpoint targeting agent and at least one lymphodepleting agent. In another embodiment, the method comprises administering to the patient the population of cells (i) and (ii) and at least one Th-1 adjuvant and at least one IL-2 family cytokine and at least one immune checkpoint targeting agent and at least one lymphodepleting agent. In another embodiment, the method comprises administering to the patient the population of cells (i), (ii) and (iii) and at least one Th-1 adjuvant and at least one IL-2 family cytokine and at least one immune checkpoint targeting agent and at least one lymphodepleting agent. In another embodiment, the method comprises administering to the patient the population of cells (i), (ii), (iii) and (iv) and at least one Th-1 adjuvant and at least one IL-2 family cytokine and at least one immune checkpoint targeting agent and at least one lymphodepleting agent. In another embodiment, the method comprises administering to the patient the population of cells (ii) and (iii) and at least one Th-1 adjuvant and at least one IL-2 family cytokine and at least one immune checkpoint targeting agent and at least one lymphodepleting agent. In another embodiment, the method comprises administering to the patient the population of cells (iii) and (iv) and at least one Th-1 adjuvant and at least one IL-2 family cytokine and at least one immune checkpoint targeting agent and at least one lymphodepleting agent. In another embodiment, the method comprises administering to the patient the population of cells (i) and (iv) and at least one Th-1 adjuvant and at least one IL-2 family cytokine and at least one immune checkpoint targeting agent and at least one lymphodepleting agent.

In another embodiment, the method comprises administering to the patient populations of cells (i) and at least one Th-1 adjuvant and at least one IL-2 family cytokine and at least one immune checkpoint targeting agent and at least one lymphodepleting agent. In another embodiment, the method further comprises administering to the patient populations of cells (ii), (iii) and/or (iv). In another embodiment, the method comprises administering to the patient populations of cells (ii) and at least one Th-1 adjuvant and at least one IL-2 family cytokine and at least one immune checkpoint targeting agent and at least one lymphodepleting agent. In another embodiment, the method further comprises administering to the patient populations of cells (i), (iii) and/or (iv). In another embodiment, the method comprises administering to the patient populations of cells (iii) and at least one Th-1 adjuvant and at least one IL-2 family cytokine and at least one immune checkpoint targeting agent and at least one lymphodepleting agent. In another embodiment, the method further comprises administering to the patient populations of cells (i), (ii) and/or (iv). In another embodiment, the method comprises administering to the patient populations of cells (iv) and at least one Th-1 adjuvant and at least one IL-2 family cytokine and at least one immune checkpoint targeting agent and at least one lymphodepleting agent. In another embodiment, the method further comprises administering to the patient populations of cells (i), (ii) and/or (iii).

Accordingly, in one preferred embodiment of the present invention, the method comprises administering to the patient the population of cells (i), the population of cells (ii), the population of cells (iii) and the population of cells (iv) and at least one Th-1 adjuvant and at least one IL-2 family cytokine and at least one immune checkpoint targeting agent and at least one lymphodepleting agent. This administration regime has provided for particularly successful results in the Examples.

Furthermore, in one embodiment, the populations of cells (i) to (iv) can be administered to said patient in a single composition, simultaneously or within 24 hours. In one embodiment, the populations of cells (i) to (iv) can be administered to said patient within 12-24 hours. In another embodiment, the populations of cells (i) to (iv) can be administered to said patient within less than 12 hours. In a further embodiment, the populations of cells of (i) to (iv) can be administered in a ratio of number of cells (i):(ii):(iii):(iv) of about 0.01-20:0.01-20:0.01-20:0.01-20. In an even more preferred embodiment, the ratio of number of cells (i):(ii):(iii):(iv) of about 0.1-10:0.1-10:0.1-10:0.1-10. In one preferred embodiment, the ratio of number of cells of (i):(ii):(iii):(iv) is about 1:1:1:1. In the examples, equal amounts of cells are used. Corresponding ratios are used in case less than four populations of cells are used. For example, in case two populations of cells are used, such as (i) and (iv) as an example, the ratio of number of cells (i):(iv) is about 0.01-20:0.01-20 or about 0.1-10:0.1-10 or about 1:1.

In another embodiment of the present invention, the cancer is selected from melanoma, renal cancer, prostate cancer, breast cancer, colon cancer, lung cancer, bone cancer, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioma, pituitary adenoma, Kaposi's sarcoma, epidermoid cancer, squamous cell cancer, T-cell lymphoma, environmentally induced cancers including those induced by asbestos, esophageal cancer, liver cancer, refractory or recurrent malignancies, metastatic cancers, and combinations of said cancers.

In one specific embodiment, the cancer(s) or tumor(s) are poorly immunogenic. In one specific embodiment, the tumor(s) are cancer tumor(s) or cancerous tumor(s). In one specific embodiment, the tumor(s) are poorly immunogenic. In another embodiment, the tumor(s) are not immunogenic at all. “Immunogenic tumors” are mainly characterized by an infiltrate of CD8+ cells that reach the center of the tumor. In non-immunogenic tumors, T cells are typically detectable in the invasion front, but these cannot penetrate into the center of the tumor. For example, a poorly immunogenic tumor is classified as infiltrated-excluded (I-E) tumor immune microenvironment (TIME). I-E TIMEs typically have CTLs localized along the border of the tumor mass in the invasive margin or ‘caught’ in fibrotic nests. I-E TIMEs are associated with various epithelial cancers such as colorectal carcinoma (CRC), melanoma and pancreatic ductal adenocarcinoma (PDAC). Accordingly, examples of poorly immunogenic tumor include epithelial cancers such as colorectal carcinoma (CRC), melanoma and pancreatic ductal adenocarcinoma (PDAC).

As used herein, the term “patient” includes any human or non-human animal. In a preferred embodiment, the subject is a human or non-human mammal, more preferably a human.

In one embodiment, the patient is human. In another embodiment, the patient is an animal.

In one embodiment, the administration regimen of the first and second aspects of the invention may be performed once. In another embodiment, the administration regimen of the first and second aspects of the invention may be may be repeated 1, 2, 3, 4, or more times. For example, the administration regimen may be repeated after 1 week or more, 2, 3 or 4 weeks or more, or 1, 2, 3, 4, 5, or 6 months or more after the population(s) of cells were administered.

In another embodiment, the invention relates to a method of preventing or treating cancer in a patient, the method comprising administering to the patient an effective amount of at least two of the following populations of cells (i) to (iv):
(i) a population of lymphokine-activated killer cells (LAKs),
(ii) a population of cytokine-induced killer cells (CIKs),
(iii) a population of γδ-T-cells,
(iv) a population of tumor-specific T-cells (CTLs),
wherein the population of cells in (i) to (iv) are derived from autologous cells from said patient or from allogeneic cells from a donor.

In a further alternative embodiment, the invention relates to a method of preventing or treating cancer in a patient, the method comprising administering to the patient an effective amount of:
at least one of the populations of cells of (i) to (iv):
(i) a population of lymphokine-activated killer cells (LAKs),
(ii) a population of cytokine-induced killer cells (CIKs),
(iii) a population of γδ-T-cells
(iv) a population of tumor-specific T-cells (CTLs),
wherein the cells in (i) to (iv) are derived from autologous cells from said patient or from allogeneic cells from a donor,
and
wherein the method further comprises administering to the patient an effective amount of:
(v) the at least one Th-1 adjuvant, and/or
(vi) the at least one IL-2 family cytokine, and/or
(vii) the at least one immune checkpoint targeting agent, and/or (viii) the at least one lymphodepleting agent.

In a further alternative embodiment, the invention relates to a method of preventing or treating cancer in a patient, the method comprising administering to the patient an effective amount of:
at least one of the populations of cells of (i) to (iv):
(i) a population of lymphokine-activated killer cells (LAKs),
(ii) a population of cytokine-induced killer cells (CIKs),
(iii) a population of γδ-T-cells
(iv) a population of tumor-specific T-cells (CTLs),
wherein the cells in (i) to (iv) are derived from autologous cells from said patient or from allogeneic cells from a donor,
and
wherein the method further comprises administering to the patient an effective amount of:
(v) the at least one Th-1 adjuvant, and
(vi) the at least one IL-2 family cytokine, and
(vii) the at least one immune checkpoint targeting agent, and
(viii) the at least one lymphodepleting agent.

The term “effective amount” in the context of the administration of a therapy to a patient refers to the amount of a therapy that achieves a desired prophylactic or therapeutic effect.

For the methods of preventing or treating disclosed herein, the same embodiments apply as for the other aspects of the invention and in particular the medicaments for use herein.

In a further aspect of the present invention, it is provided a pharmaceutical composition, a kit or a kit-of-parts comprising the populations of cells of the first and second aspects and at least one or more further agents of the second aspect of the present invention.

The invention relates, in a third aspect, to a pharmaceutical composition, kit or kit-of parts comprising at least two of the following populations of cells (i) to (iv):
(i) a population of lymphokine-activated killer cells (LAKs),
(ii) a population of cytokine-induced killer cells (CIKs),
(iii) a population of γδ-T-cells,
(iv) a population of tumor-specific T-cells (CTLs),
wherein the cells in (i) to (iv) are derived from autologous cells from a patient or from allogenic cells from a donor.

In one embodiment, the pharmaceutical composition, kit or kit-of parts comprises. three or four of (i) to (iv).

In one embodiment, the pharmaceutical composition, kit or kit-of parts further comprises one or more of the following:
(v) at least one Th-1 adjuvant,
(vi) at least one IL-2 family cytokine,
(vii) at least one immune checkpoint targeting agent,
(viii) at least one lymphodepleting agent.

The invention relates, in a fourth aspect, to a pharmaceutical composition, kit or kit-of parts comprising at least one of the following populations of cells (i) to (iv):
(i) a population of lymphokine-activated killer cells (LAKs),
(ii) a population of cytokine-induced killer cells (CIKs),
(iii) a population of γδ-T-cells,
(iv) a population of tumor-specific T-cells (CTLs),
wherein the cells in (i) to (iv) are derived from autologous cells from a patient or from allogenic cells from a donor,
and wherein the pharmaceutical composition, kit or kit-of parts further comprises the following (v) to (viii):
(v) at least one Th-1 adjuvant,
(vi) at least one IL-2 family cytokine,
(vii) at least one immune checkpoint targeting agent, and
(viii) at least one lymphodepleting agent.

In one embodiment, the pharmaceutical composition, a kit or a kit-of-parts of the third or fourth aspect, the pharmaceutical composition, kit or kit-of parts comprises at least two of the populations (i) to (iv) and the at least two populations of cells are comprised in a single composition or in separate compositions.

These compositions may be comprised in a solution, such as an aqueous solution, or dried or freeze-dried, and optionally further containing pharmaceutically acceptable excipients.

In another embodiment, the pharmaceutical composition, a kit or a kit-of-parts of the third or fourth aspect, the pharmaceutical composition, kit or kit-of parts comprises at least two of the populations (i) to (iv) and the at least two populations of cells are in a single container or in separate containers.

In another embodiment, the pharmaceutical composition, kit or kit-of parts comprises at least two of the populations (i) to (iv) and the at least two populations of cells are comprised in a pharmaceutical composition further comprising at least one pharmaceutically acceptable excipient.

The amount of the respective agent(s) of the invention such as the populations of cells, which will be effective in the treatment of a particular disorder or condition, will depend on the nature of the disorder or condition, and can be determined by standard clinical techniques. Preferred amounts and dosages are provided for the respective agents above. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Unless indicated otherwise for specific agents used in the present invention above, administration can be systemic or local.

Unless indicated otherwise for specific agents used in the present invention above, in some embodiments, it may be desirable to administer the pharmaceutical compositions locally to the area in need of treatment. This may be achieved by, for example, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Further, administration can be by direct injection at the site of the tumor or cancer.

In yet another embodiment, unless indicated otherwise for specific agents used in the present invention above, the agents can be delivered via a controlled release system. In one embodiment, a pump may be used. Further, a controlled release system can be placed in proximity of the therapeutic target (in particular cancer or tumor), thus requiring only a fraction of the systemic dose.

In one embodiment, routes of administration for the pharmaceutical composition as described above, include, but are not limited to those: intravenous, intratumoral, and/or intra-arterial. In a preferred embodiment, the pharmaceutical composition described herein is delivered intravenously. In another preferred embodiment, the pharmaceutical composition described herein is delivered by inhalation. In certain embodiments, the pharmaceutical composition described herein is delivered intra-arterially. In certain embodiments, the pharmaceutical composition described herein is delivered intratumorally. In certain embodiments, the pharmaceutical composition described herein is delivered to a tumor draining lymph node.

The route of administration is selected independently for any of the active agents therein. For example, the route of administration for the at least one or at least two of the populations of cells of (i) to (iv) of the aspects of the invention herein may be the same or different as the route of administration for (v) the at least one Th-1 adjuvant, (vi) the at least one IL-2 family cytokine, (vii) the at least one immune checkpoint targeting agent, or (viii) the at least one lymphodepleting agent. For example, all agents may be administered by systemic administration, such as by intravenous administration. For example, all agents may be administered by local administration, such as by intratumoral administration. For example, all agents except (v) the at least one Th-1 adjuvant may be administered by systemic administration, such as by intravenous administration, and (v) the at least one Th-1 adjuvant may be administered by inhalation.

Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of the respective agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The agents of the invention, especially where these agents are not cells, can be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

In another embodiment, the pharmaceutical composition, kit or kit-of-parts of the third or fourth aspect, the population(s) of cells are in a buffered solution. In another embodiment the pharmaceutical composition, a kit or a kit-of-parts of the third aspect, the patient is suffering from cancer.

In another embodiment of the present invention, the pharmaceutical composition, kit or kit-of parts of the third or fourth aspect is further characterized by the features of any of those disclosed in the first and/or the second aspect and/or further aspect(s).

“About” is understood as the indicated value ±10% or ±5%.

### Examples

### Methods Used in the Examples

### Statistics

Statistical analyses and data plotting were performed with R⁶⁴ and Graphpad⁶⁵. Statistical tests used are indicated in figure legends or throughout the text.

### Ethics

All human subject research (PDX generation, PBMC donation) was performed in accordance with approved protocols by the local ethics committee and the Declaration of Helsinki.

### Cell Lines

The murine melanoma cell line B16F10 was kindly provided by Hans Schlößer (University of Cologne, Germany), the Hodgkin lymphoma cell lines L428, L540, L1236 and KMH2 were kindly provided by Hinrich Hansen (University of Cologne, Germany). The human lung cancer cell lines H1975 and H441 were obtained from ATCC (Manassas, VA). JimT1 was obtained from DSMZ (Leipzig, Germany). The Kras/p53-null (KP) and MCA-induced/p53-null (MCA/p53) sarcoma cell lines were kindly provided by David Kirsch (Duke University, NC). The murine lung cancer cell line KP 938.3, generated by culturing Kras^{LSL-G12D} p53^{fl/fl} tumors in-vitro, was kindly provided by Christian Reinhardt (University of Essen, Germany). Some cell lines were transduced to stably express firefly luciferase for cell viability monitoring by luminescence⁶⁶.

All cell lines were cultured in a 50%/50% v/v mixture of RPMI-1640 (Gibco, Carlsbad, CA) and DMEM-F12 (Gibco) supplemented with 10% fetal bovine serum (FBS) and 100 U/ml Penicillin (Gibco) and 100 μg/ml Streptomycin (Gibco). All cell lines were cultured, and all in-vitro assays performed at 37° C. and 5% CO2. Cell lines were regularly tested for mycoplasma contamination.

### Tripartite IMMUNOTHERAPY

Tripartite immunotherapy (TRI-IT) consists of three treatment components. The first component is adoptive cellular therapy of a combined cell therapy product consisting of lymphokine activated killer cells (LAKs), cytokine induced killer cells (CIKs), Vγ9Vδ2-T-cells and in vitro enriched, tumor-specific T-cells (CTLs). Cells were given in equal proportion as part of the combined treatment, meaning a quarter of the given number of cells consisted of each cell type. The cell dose was heuristically determined and 1×10⁷ cells in up to 200 μl of PBS were injected i.p. per dose. To support engraftment, cellular therapy was followed by 5 daily doses of 1×10⁵ IE recombinant Interleukin (IL)-2 (Aldesleukin, Novartis, Basel, Switzerland) in up to 100 μl PBS s.c. The second component is systemic immune checkpoint inhibition by i.p. injection of 5 mg/kg of either anti-mouse aPD1 antibody (clone RMP1-14, BioXCell, Lebanon, NH) or clinical-grade Nivolumab (Bristol-Myers-Squibb, New York, NY) in 100 μl PBS per dose. Two doses per week were given. The third component is local immune stimulation by a mix of agonists against TLR3, TLR7 and TLR9. The agonists used were Poly I:C (Millipore, 1.25 mg/kg/BW), Gardiquimod (Cayman Chemical, Ann Arbor, MI, 1.25 mg/kg/BW) and ODN-2395 (5′-tcgtcgttttcggcgcgcgccg-3′ with phosphorothioate bonds, synthesized by IDT, Coralville, IA, 1.25 mg/kg/BW). These were given twice weekly diluted in PBS intratumorally (or peritumorally if tumors were too small to be directly injected) per dose. Unless indicated otherwise, only the right tumor was injected to model injection of only one lesion in a patient. In one experiment TLR agonists were given inhaled at similar concentrations after isoflurane anesthesia.

If lymphodepletion was given, it consisted of clinical-grade cyclophosphamide (HEXAL, Holzkirchen, Germany, 200 mg/kg/BW) and clinical-grade fludarabine (Sanofi Genzyme, Paris, France, 40 mg/kg/BW) given i.p. in 100 μl PBS 24 hours before treatment began. If an experimental group included only a subset of treatments, the other treatments were substituted with appropriate controls. These were either identically applied PBS injections or appropriate IgG controls for aPD1-antibody treatment (Isotype control rat IgG2a, κ (BioXCell) for murine a-PD1 and clinical-grade human IgG (Octagam, octapharma, Lachen, Switzerland) for Nivolumab.

### Animal Experiments

Experiments were performed in accordance with FELASA recommendations. The protocol was approved by the local ethics committee. Mice were housed and all experiments performed in a sterile environment. Mice were fed, given water and monitored daily for health, and cages were changed weekly.

### In Vivo Tumor Growth Experiments

For the subcutaneous B16F10 melanoma model, 8-12-week-old C57BL/6J mice were inoculated subcutaneously with 5×10⁵ B16F10 melanoma cells in each flank in 100 μl in PBS. Treatment was initiated on day 10 after inoculation on which tumors had an average size of 50-100 mm³. For the subcutaneous KP lung cancer melanoma model, 8-12-week-old C57BL/6J mice were inoculated subcutaneously with 5×10⁶ KP938.3 cells in each flank in 100 μl in PBS. Treatment was initiated on day 10 after inoculation on which tumors had an average size of 50-100 mm³. For the subcutaneous sarcoma models, 8-12-week-old 129/SvJ mice were inoculated subcutaneously with 2×10⁵ MCA/p53 or KP sarcoma cells in each flank in 100 μl in PBS. Treatment was initiated on day 10 after inoculation on which tumors had an average size of 50-100 mm³. Tumor growth was measured twice weekly by caliper measurement of the longest diameter I and an orthogonal measurement s. Tumor volume was estimated with the following formula:

$$\frac{{s}^{2} * l}{2}$$

The fold change of tumor growth was calculated by dividing the tumor volume of a specific measurement by the initial tumor volume to account for differences in initial tumor volume⁶⁷.

### Autochthonous NSCLC Model

As a well-established autochthonous model for KRAS-mutant lung adenocarcinomas, we used Kras^{LSL-G12D} p53^{fl/fl} (KP) mice as described previously⁴⁸. Tumor induction was performed in 6-to-8-week old KP mice using a type 2 alveolar epithelial cell-specific Adenovirus-Cre (Ad5mSPC-Cre from Viral Vector Core, University of Iowa/Anton Bern, NKI). Mice were anesthetized with Ketamine/Xylazine (100 mg/kg body weight and 10 mg/kg body weight, respectively, i.p.) and 2×10⁷ pfu Adenovirus-Cre were administered intranasally, as previously described⁴⁸. Four weeks after virus inhalation, lungs were scanned by μCT to confirm tumor formation. Subsequently, tumor progression was monitored by weekly μCT scans with a LaTheta LCT-100 small animal μCT (Hitachi Aloka Instruments, Tokyo, Japan). CT images of the whole lung were taken at 0.3 mm intervals and analysed using InVesalius 3.0 software (Amsterdam, Netherlands).

### Autologous Humanized PDX Models

Tumor material was obtained from consenting non-small cell lung cancer (NSCLC) patients. To establish 1^{st} generation PDX, the tumor specimen was transported sterile directly from surgery to the animal facility in RPMI-1640 (Gibco) supplemented with 10% FCS and Penicillin-Streptomycin, where it was dissected into 3×3×3 mm large pieces. Subcutaneous pockets were prepared in both flanks of 8 to 12-week-old NSG (NOD.Cg-Prkdcscid Il2rgtm1Wjl/SzJ) mice in general anesthesia and one piece was tumor tissue was inserted into the prepared pocket. At the end of the procedure, the skin is closed with adhesive and mice received adequate post-surgical care with daily wound controls and analgesia. Once tumors were established, growth was monitored. When tumors reached a size of about 1 cm in their largest diameter, they were transplanted as described above into the next generation of mice. A PDX model was considered stable, when it had been transplanted at least 3 times and the 4^{th} generation was the earliest generation, in which mice were used for experiments. In parallel, we generated cell lines from tumors by mashing one 3×3×3 mm large tumorfragment with 3 parallel sterile scalpel blades and transferring the resulting mashed tissue into cell culture plates filled with a 50%/50% v/v mixture of RPMI-1640 (Gibco) and DMEM-F12 (Gibco) supplemented with 10% Fetal bovine serum (FBS) and Penicillin-Streptomycin. We used large cell culture plates filled with at least 50 ml of medium. Cells were left untouched for 3 days. On day 3, half the medium was exchanged carefully for fresh medium. Cultures were monitored for emergence of adherent tumor cell clusters. Once these were identified, all medium was removed, and culture plates were washed with PBS before continuing the culture with new medium. Cells were only split by trypsinization if they were fully confluent. We observed a reduction of proliferating fibroblasts and dominance of tumor cells over time in successful cultures, whereas only fibroblasts proliferated after some time in unsuccessful cultures and ultimately stopped proliferating as expected.

For tumor growth experiments, mice were humanized with 2.5×10⁶ autologous PBMCs i.p. on the same day as they were transplanted with a PDX fragment as described above. Treatment (as described above in “Tripartite Immunotherapy”) was initiated on day 10 to 16 depending on the PDX model once tumors reached an average size of 50-100 mm³.

### Long-Term Tumor Control and Re-Challenge Experiments

Mice were inoculated subcutaneously with one B16F10 or KP938.3 tumor as described above. Two full treatment cycles were given starting on day 10 after inoculation. These consisted of lymphodepletion followed 24 hours later by adoptive cellular therapy followed by 3 days of s.c. IL-2 to support engraftment. After adoptive cellular therapy, systemic immune checkpoint inhibition targeting PD1 and local immunotherapy with the TLR-agonist mix was given twice weekly as described above. Another, identical treatment cycle was given on day 27. All treatment was stopped on day 49. On day 60 mice were re-challenged with B16F10 or KP938.3 cells, respectively. No further treatment was given, and mice were observed for tumor growth until day 120.

### Synergy of Adoptive Cellular Components In Vivo

For studying the synergy of adoptive cellular therapy components mice were either treated with 1×10⁷ cells of combined adoptive cellular therapy or with 1×10⁷ of each component, LAKs, CIKs, γδT-cells or CTLs. Lymphodepletion was applied in the B16F10 model.

### Evaluation of Tri-IT Toxicity

Toxicity of TRI-IT was evaluated in the subcutaneous B16F10 melanoma model. Mice were divided into 4 groups: TRI-IT+B16F10 inoculation, IgG/PBS control+B16F10 inoculation, TRI-IT+mock tumor inoculation (PBS only) and IgG/PBS control+mock tumor inoculation (PBS only). Mice were weighed twice weekly starting before treatment initiation (day 10). On day 24, mice were euthanized, organs harvested, weighed, and fixed for immunohistochemistry in 4% PBS-buffered formalin.

### Cellular Therapy Components

Peripheral blood mononuclear cells (PBMCs) were purified either from donor buffy coats (allogenic PDX models) or patient blood samples (autologous PDX models) by standard Ficoll-Paque density gradient centrifugation. Mouse splenocytes were generated by harvesting spleens from syngeneic female mice by straining spleens through a sterile 40-mm cell strainer (BD Falcon). All cellular therapy components were cultured in cell expansion medium which consisted of a 50%/50% v/v mixture of RPMI-1640 (Gibco) and DMEM-F12 (Gibco) supplemented with 10% fetal bovine serum (FBS), 100 U/ml Penicillin (Gibco), 100 μg/ml Streptomycin (Gibco) and 50 μM β-2-Mercaptoethanol (Roth). Human LAKs were generated by culturing PBMCs at a density of 1.25×10⁶ cells/ml in cell expansion medium supplemented with 1000 IE/ml recombinant human IL-2 (Aldesleukin, Novartis) for 7 days. Half the media was exchanged, and culture volume appropriately increased to maintain the initial cell density every 3 days or earlier if needed and cells were harvested on day 7 of culture.

Human γδT-cells were enriched by culturing PBMCs at a density of 1.25×10⁶ cells/ml in cell expansion medium supplemented with 1000 IE/ml recombinant human IL-2 (Aldesleukin, Novartis) and 5 μM clinical-grade zoledronic acid (HEXAL). Half the media was exchanged, and culture volume appropriately increased to maintain the initial cell density every 3 days or earlier if needed. Zoledronic acid was not added anymore beyond day 3. γδT-cells were harvested on day 14 of culture. Human CIKs were generated by culturing PBMCs at a density of 1.25×10⁶ cells/ml in cell expansion medium supplemented with 1000 IE/ml recombinant human IFNγ (Miltenyi Biotech) for 24 hours. Next, media was exchanged for cell expansion medium supplemented with 300 IE/ml recombinant human IL-2 (Aldesleukin, Novartis). Half the media was exchanged, and culture volume appropriately increased to maintain the initial cell density every 3 days or earlier if needed. CIKs were harvested on day 14 of culture.

Human tumor-specific CTLs were enriched by an adaption of the method of Dijkstra et al.²⁴. First, target tumor cells were pretreated with 50 or 100 μg/ml Mitomycin C (Medac) and 1000 IE/ml recombinant human IFNγ (Miltenyi Biotech, Bergisch-Gladbach, Germany) in cell expansion medium for 24 h in a regular cell culture incubator (37° C., 5% CO₂) and then washed 4 times with PBS. The next day, PBMCs were co-cultured with the target tumor cells at a density of 1.25×10⁶ PBMCs/ml in cell expansion medium supplemented with 150 IE/ml recombinant human IL-2 (Aldesleukin, Novartis), 10 ng/ml recombinant human IL-7 (Miltenyi Biotech), 5 μg/ml anti-human, anti-CD28 (clone CD28.2, Biolegend) and 20 μg/ml anti-human anti-PD1-antibody (Nivolumab, Bristol Myers Squibb). Target tumor cells were added at a 10:1 ratio (PBMCs to tumor cells). Half the media was exchanged, and culture volume appropriately increased to maintain the initial cell density every 3 days or earlier if needed. With each media exchange, media was only supplemented with 150 IE/ml recombinant human IL-2 (Aldesleukin, Novartis) and 20 μg/ml anti-human anti-PD1-antibody (Nivolumab, Bristol Myers Squibb). CTLs were harvested on day 14 of culture.

Murine LAKs were generated by culturing syngeneic, female splenocytes at a density of 1.25×10⁶ cells/ml in cell expansion medium supplemented with 6000 IE/ml recombinant human IL-2 (Aldesleukin, Novartis). For 7 days. Half the media was exchanged, and culture volume appropriately increased to maintain the initial cell density every 3 days or earlier if needed and cells were harvested on day 7 of culture.

For murine γδT-cell enrichment, culture dishes were coated with anti-mouse anti-γδTCR (clone UC7-13D5, Biolegend) at 4° C. for 24 hours. The coated culture dishes were washed 4 times with PBS. γδT-cells were enriched by culturing syngeneic, female splenocytes at a density of 1.25×10⁶ cells/ml in cell expansion medium supplemented with 100 IE/ml recombinant human IL-2 (Aldesleukin, Novartis) on anti-mouse anti-γδTCR-coated culture plates for 72 h. After that cells were harvested, transferred to new culture dishes and expanded at a density of 1.25×10⁶ cells/ml in cell expansion medium supplemented with 100 IE/ml recombinant human IL-2 (Aldesleukin, Novartis). Half the media was exchanged, and culture volume appropriately increased to maintain the initial cell density every 3 days or earlier if needed. γδT-cells were harvested on day 10 of culture.

Murine CIKs were generated by culturing syngeneic, female splenocytes at a density of 1.25×10⁶ cells/ml in cell expansion medium supplemented with 1000 IE/ml recombinant murine IFNγ (Miltenyi Biotech) for 24 hours. In parallel, culture dishes were coated with anti-mouse anti-CD3 (clone 145-2C11, Biolegend) at 4° C. for 24 hours. The coated culture dishes were washed 4 times with PBS. Next, splenocytes were transferred to the coated culture dishes and media was exchanged for cell expansion medium supplemented with 300 IE/ml recombinant human IL-2 (Aldesleukin, Novartis). After 72 h of incubation on the coated culture dishes, CIKs were transferred to new culture dishes in fresh for cell expansion medium supplemented with 300 IE/ml recombinant human IL-2 (Aldesleukin, Novartis). Half the media was exchanged, and culture volume appropriately increased to maintain the initial cell density every 3 days or earlier if needed. CIKs were harvested on day of culture.

Murine, tumor-specific CTLs were enriched similar to human CTLs. First, target tumor cells were pretreated with 50 or 100 μg/ml Mitomycin C (Medac) and 1000 IE/ml recombinant murine IFNγ (Miltenyi Biotech) in cell expansion medium for 24 h in a regular cell culture incubator (37° C., 5% CO₂) and then washed 4 times with PBS. The next day, syngeneic, female splenocytes were co-cultured with the target tumor cells at a density of 1.25×10⁶ PBMCs/ml in cell expansion medium supplemented with 10 IE/ml recombinant human IL-2 (Aldesleukin, Novartis), ng/ml recombinant human IL-7 (Miltenyi Biotech), 2 μg/ml anti-mouse anti-CD28 (clone 37.51, Biolegend) and 20 μg/ml anti-mouse anti-PD1-antibody (clone RMP1-14, BioXCell). Target tumor cells were added at a 10:1 ratio (PBMCs to tumor cells). Half the media was exchanged, and culture volume appropriately increased to maintain the initial cell density every 3 days or earlier if needed. With each media exchange, media was only supplemented with 10 IE/ml recombinant human IL-2 (Aldesleukin, Novartis) and 20 μg/ml anti-mouse anti-PD1-antibody (clone RMP1-14, BioXCell). CTLs were harvested on day 10 of culture.

All cells for adoptive cellular therapy were used fresh without freezing them after expansion. The phenotype of cells was confirmed by FACS.

### Cellular Depletions

For cellular depletion studies mice were treated with full TRI-IT, but selected immune cells were depleted with the following antibodies: anti-mouse anti-CD4 (clone, GK1.5, BioXCell), anti-mouse anti-CD8a (clone 2.43, BioXCell), anti-mouse anti-NK1.1 (clone PK136, BioXCell), anti-mouse anti-γδTCR (clone UC7-13D5, BioXCell), anti-mouse anti-CSF1R (clone AFS98, BioXCell), anti-human anti-CD4 (clone OKT-4, BioXCell) and anti-human anti-CD8a (clone OKT-8, BioXCell). All depleting antibodies were given at day −1, 0 and 1 of the first treatment day and twice weekly after that at a dose of 100 μg in PBS i.p. Successful depletions of targeted immune cells were validated by FACS analysis of spleens.

### Immunohistochemistry

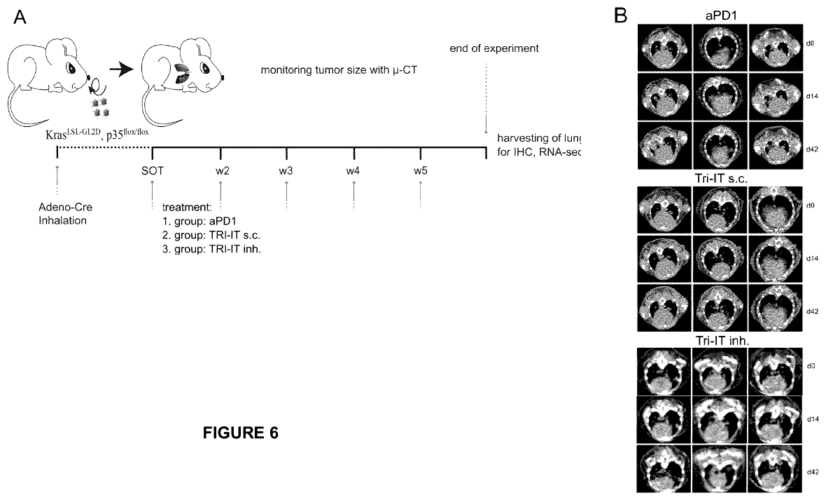

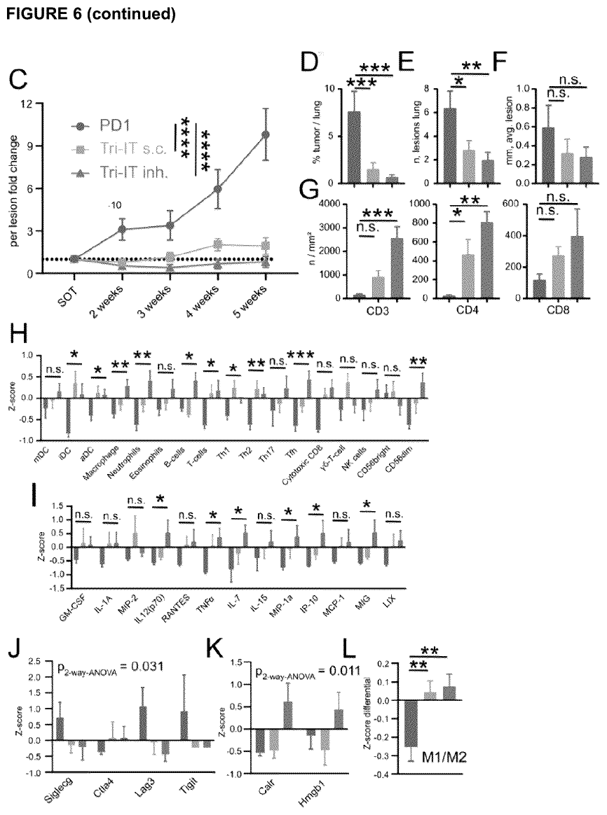

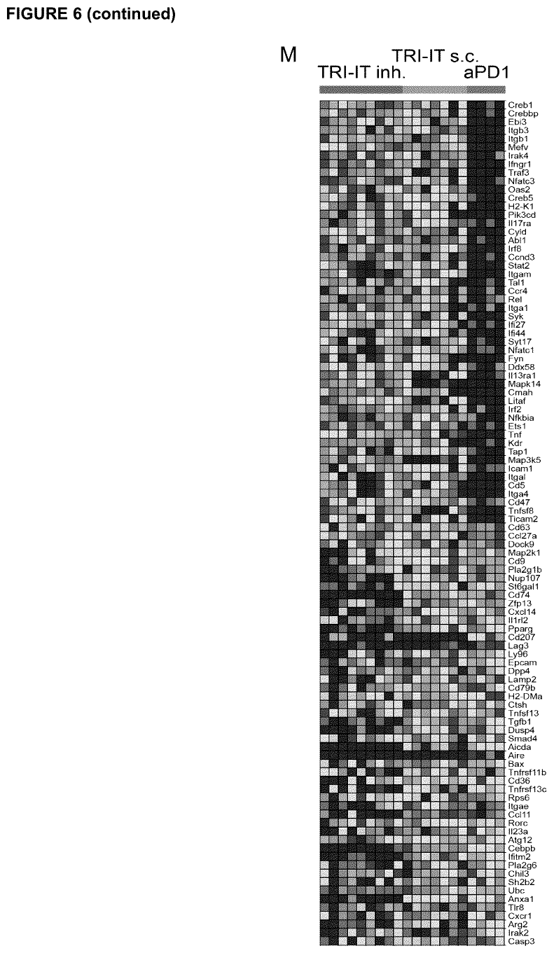

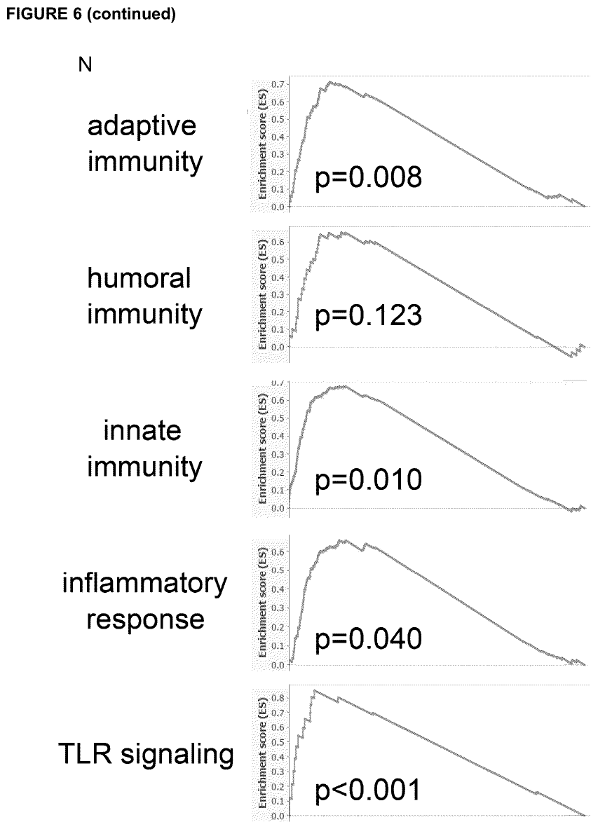

Tissues were harvested and fixed in 4% PBS-buffered formalin before paraffin embedding. Subsequently, 3 μm tissue sections were deparaffinized and immunohistochemistry (IHC) or hematoxylin & eosine staining (H&E) was performed using the LabVision Autostainer-480S (Thermo Scientific, Waltham, MA) or the Leica Bond Max system (Leica microsystems, Wetzlar, Germany) for anti-mouse or anti-human IHC, respectively. For mouse tissues, primary anti-mouse antibodies against CD3 (Thermo Fisher, RM-9107-S), CD4 (Abcam, Cambidge, UK, EPR19514) and CD8 (Abcam polyclonal, ab203035) were used. For human tissues, primary anti-human antibodies against CD3 (clone SP7, Thermo Fischer), CD4 (clone 4B12, thermo Fisher) CD8 (clone C8/144B, Dako, Glostrup, Denmark) and Ki67 (clone SP6, Cell Marque, Rocklin, CA) were used. Detection of primary anti-mouse antibodies was performed with the Secondary-Histofine-Simple-Stain (SHSS) antibody detection kit (Medac, Wedel, Germany). Slides were scanned by the Panoramic-250 slide scanner (3D Histech) or the SCN400 slide scanner (Leica). Scanned slides were digitally viewed and analyzed using the software Aperol ImageScope (Leica) or Panoramic Viewer (3D Histech, Budapest, Hungary). For quantification of CD3+, CD4+ and CD8+ cells in the autochthonous KP model (FIG. 6), cells were counted in three randomly chosen fields of 0.05 m², averaged and multiplied by 20 to get positive cells per mm². For quantification of T− cells in lung and liver tissue in the toxicity evaluation experiment (FIG. S9), CD3+ cells were counted in 5 randomly chosen complete viewing fields at 40× in Aperol ImageScope (Leica) and averaged. To measure intestinal T-cell infiltration, CD3+ cells were counted in 30 randomly chosen transverse cross sections of intestinal villi. Investigators were blinded to the experimental group during quantifications.

### Multiplex Analysis of Cytokines

For multiplexed quantification of 41 human cytokines, chemokines, and growth factors, we used Luminex xMAP technology. The multiplexing analysis was performed using the Luminex™ 100 system (Luminex, Austin, TX) by Eve Technologies Corp. (Calgary, Alberta). Forty-one markers were simultaneously measured in the samples using a MILLIPLEX Human Cytokine/Chemokine 41-plex kit (Millipore, Burlington, MA) according to the manufacturer's protocol. The 41-plex consisted of EGF, Eotaxin, FGF-2, Flt-3 ligand, Fractalkine, G-CSF, GM-CSF, GRO, IFN-α2, IFN-γ, IL-10, IL-12 (p40), IL-12 (p70), IL-13, IL-15, IL-17A, IL-1ra, IL-1α, IL-1β, IL-2, IL-3, IL-4, IL-5, IL-6, IL-7, IL-8, IL-9, IP-10, MCP-1, MCP-3, MDC (CCL22), MIP-1a, MIP-1b, PDGF-AA, PDGF-AB/BB, RANTES, TGFα, TNF-α, TNF-β, VEGF, and sCD40L. The assay sensitivities of these markers range from 0.4-26.3 pg/mL for the 41-plex. Individual analyte values are available in the MILLIPLEX protocol.

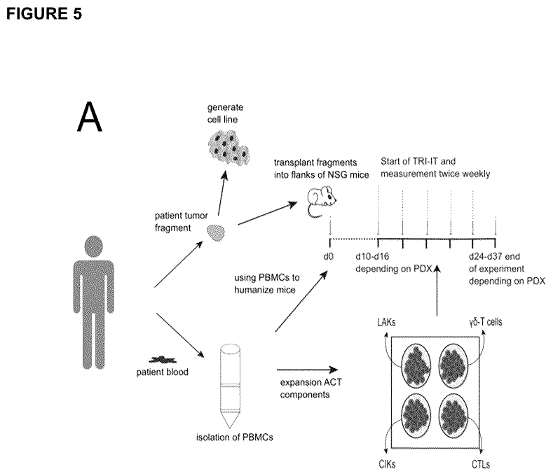

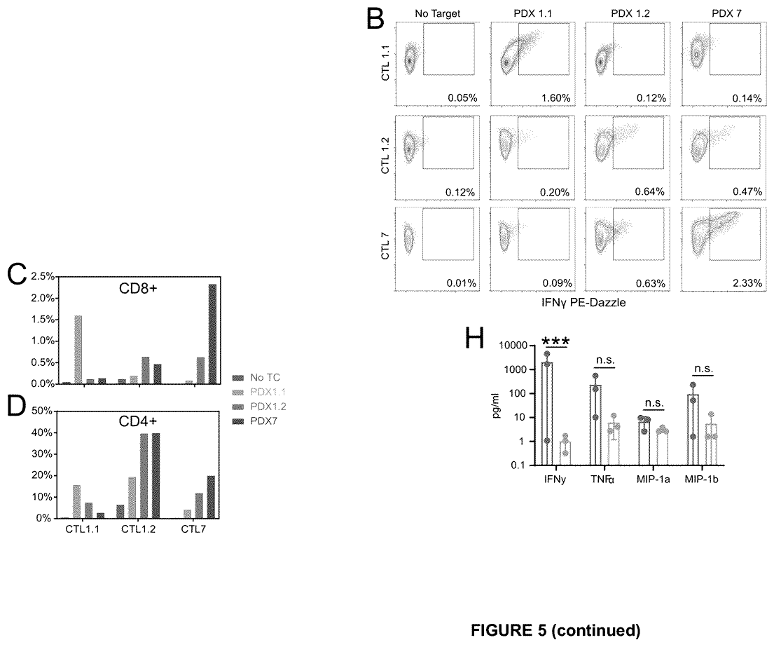

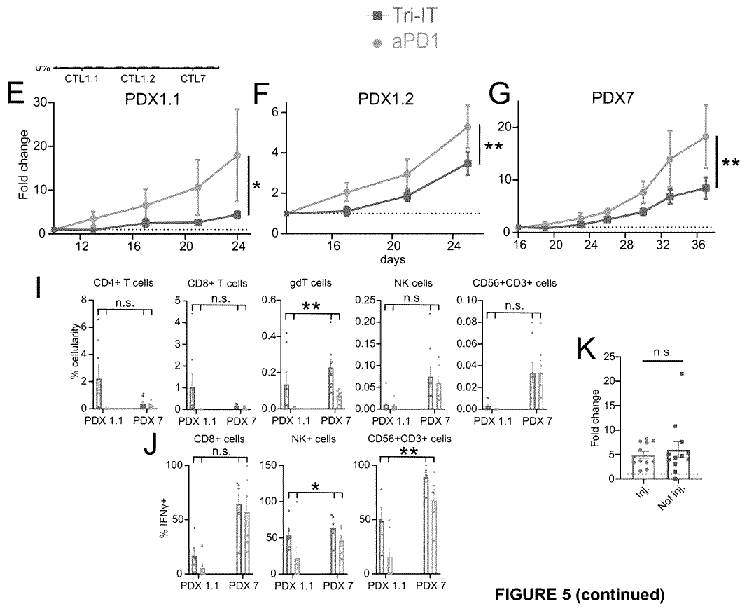

Similarly, we performed multiplexed quantification of 32 murine cytokines, chemokines, and growth factors. Thirty-two markers were simultaneously measured in the samples using a MILLIPLEX Mouse Cytokine/Chemokine 32-plex kit (Millipore) according to the manufacturer's protocol. The 32-plex consisted of Eotaxin, G-CSF, GM-CSF, IFNγ, IL-1α, IL-1β, IL-2, IL-3, IL-4, IL-5, IL-6, IL-7, IL-9, IL-10, IL-12 (p40), IL-12 (p70), IL-13, IL-15, IL-17, IP-10, KC, LIF, LIX, MCP-1, M-CSF, MIG, MIP-1a, MIP-1b, MIP-2, RANTES, TNFα, and VEGF. The assay sensitivities of these markers range from 0.3-30.6 pg/mL for the 32-plex. Individual analyte values are available in the MILLIPLEX protocol. As most human cytokines were not consistently detectable in humanized mice, we focused on a select few for analysis in the autologous humanized PDX model (FIG. 5), namely IFNγ, IL-2, MIP-1a and MIP-1b. Rarely, a cytokine was detectable, but its level below the limit of quantification. In these cases, we set its value at halve the limit of quantification. Z-scores of the log₁₀ of raw cytokine levels were calculated for each cytokine to generate heatmap plots with Morpheus⁷⁴. To generate volcano plots, t-tests were performed using the log₁₀ of raw cytokine levels as input and multiple testing correction was performed setting the false discovery rate to 5% and using the two-stage linear step-up procedure of Benjamini, Krieger and Yekutieli⁷⁵. To analyze the relationship between cytokine measurements and tumor growth in some experiments, PLR models were constructed using the PLS package⁷⁶ and R⁶⁴. Z-scores of the log₁₀ of raw cytokine levels were used as predictors to predict the log 2 tumor growth fold change at the end of the experiment with leave-one-out cross-validation. The number of components were selected as such, that the root mean squared error of prediction of the selected model was minimal. VIP scores were used to measure each cytokine's contribution to the prediction and a VIP score >1 was considered significant.

### Flow Cytometry

Organs or tumors of mice were harvested and manually disintegrated, first with a scalpel and then bluntly. Subsequently, cells were manually dissociated using a 40-mm cell strainer (BD, Franklin Lakes, NJ). Resulting single-cell suspensions were washed once with PBS and resuspended in FACS buffer (PBS supplemented with 2% FCS and 1 mM EDTA). Cells from cell culture experiments were washed once with PBS and resuspended in FACS buffer. Cells were first stained with the fixable viability dye Zombie UV (Biolegend, San Diego, CA) and extracellular antibodies for 30 min at 4° C. in FACS buffer. Next, cells were washed once in FACS buffer, fixed in FACS buffer supplemented with 1% formalin for 10 min at 4° C., washed again once with FACS buffer and permeabilized in FACS buffer supplemented with 0.1% Triton X-100 for 10 min at 4° C. followed by another wash with FACS buffer. Finally, cells were stained with intracellular antibodies for 30 min at 4° C. in FACS buffer. Before analysis, cells were washed twice with FACS buffer. The following extracellular anti-mouse antibodies were used for the analysis of murine cells: anti-CD45-PerCP-Cy5.5 (clone 30-F11), anti-CD3-PE-Cy7 (clone 145-2C11), anti-PD1-APC (clone 29F.1A12), anti-NK1.1-AF700 (clone PK136), anti-γδTCR-APC-Fire750 (clone GL3), anti-CD8-BV421 (clone 53-6.7), anti-CD4-BV785 (clone GK1.5) and anti-CD69-BUV737 (clone H1.2F3, BD). The following intracellular anti-mouse antibodies were used: anti-FoxP3-PE (clone 150D), anti-IFN-γ-PE-Dazzle (clone XMG1.2) and anti-Ki67-BV605 (clone 16A8). The following extracellular anti-human antibodies were used for the analysis of human cells: anti-CD56-PerCP-Cy5.5 (clone HCD56), anti-γδTCR-PE-Cy7 (clone B1), anti-CD4-APC (clone RPA-T4), anti-CD8-AF700 (clone SK1), anti-CD16-APC-Fire750 (clone 3G8), anti-CD69-BV421 (clone FN50), anti-PD1-BV605 (clone EH12.2H7), anti-CD45-BV785 (clone H130) and anti-CD3-BUV737 (clone UCHT1, BD). The following intracellular anti-human antibodies were used: anti-FoxP3-PE (clone 150D), anti-IFN-γ-PE-Dazzle (clone B27) and anti-Ki67-FITC (clone 11F6). All antibodies were obtained from Biolegend, if not indicated otherwise. Acquisition and enumeration of cells were performed on a Cytoflex LX Flow Cytometer (Beckman Coulter, Pasadena, CA). Data was analyzed using Kaluza (version 2.1, Beckman Coulter). Results are reported as frequency or geometric mean of fluorescence intensity. For analysis, gates were first set to select alive, appropriately sized, CD45+ and singleton cells, after which analysis-specific gates were applied. T-cells were defined as CD45+CD3+ cells. Tregs were defined as CD45+CD3+FoxP3+ cells. CD4+ T-cells were defined as CD45+CD3+FoxP3-CD4+ cells. CD8+ T-cells were defined as CD45+Cd3+FoxP3-CD8+ cells. Human NK cells were defined as CD45+CD3−CD56+ cells. Murine NK cells were defined as CD45+CD3-NK1.1+ cells. γδT-cells were defined as CD45+CD3+FoxP3+γδTCR+ cells. Human CD3+CD56+ cells were defined as CD45+CD3+CD56+ cells. Murine CD3+NK1.1+ cells were defined as CD45+CD3+NK1.1+ cells. All gates were placed in the same position within one experiment.

### Allogenic Humanized Xenograft Models

8 to 12 week old NSG (NOD.Cg-Prkdcscid Il2rgtm1Wjl/SzJ) mice were humanized with 1×10⁷ healthy donor PBMCs i.p. For each experiment involving adoptive cellular therapy, PBMCs used to generate or enrich cells for adoptive cellular therapy were from the same donor as those used for humanization. One week after humanization mice were inoculated subcutaneously with 5×10⁶ cancer cells (H441, H1975, JimT1, L428 or KMH2). Treatment was initiated on day 10 after tumor inoculation as described in Tripartite Immunotherapy. Successful humanization was validated at the end of each experiment by flow cytometric analysis of human CD45+ cells in spleens.

### Evaluation of Cellular Immune Response

To evaluate cytotoxicity, 1×10⁴ luciferase transduced target cells (B16F10-luc2 or KP938.3-luc2) target cells were added per well to 96 well plates in cell expansion medium. Splenocytes were thawed, washed in PBS and viable cells were counted. Next, splenocytes were added at a 30:1 ratio to target cells. Control wells without addition of splenocytes but with target cells and empty background control wells were added to each plate. Each condition was tested at least in duplicates. Luciferin (Regis Biotechnology, Morton Grove, IL) was added to each well for a final concentration of 200 μg/ml. After 24 hours luminescence was measured with a VICTOR Nivo multimode plate reader (Perkin Elmer). Relative toxicity was calculated according to the following formula:

$$1 - \frac{{Lum}_{splenocytes + tumor cells} - {Lum}_{background}}{{Lum}_{tumor cells} - {Lum}_{background}}$$

To evaluate IFNγ response after splenocyte restimulation, cells were harvested after luminescence measurement, stained for flow cytometry as described above and run on the Cytoflex LX Flow Cytometer (Beckman Coulter) for analysis. Kaluza software (version 2.1, Beckman Coulter) was used to calculate the proportion of IFNγ+ cells as part of CD4+, CD8+, γδT-cell, NK and NK1.1+CD3+ cells.

### Evaluation of Endogenous Antibody Response

B16F10 cells or KP938.3 cells were incubated with 5% serum from mice of various experimental groups or syngeneic naïve mice and the fixable viability dye Zombie UV (Biolegend) in FACS Buffer for 30 minutes at 4° C., washed twice with FACS Buffer and then incubated with PE-labeled goat anti-mouse IgG (Poly4053, Biolegend) for 30 minutes at 4° C. for detection of serum antibodies bound to tumor cells. Lastly, cells were washed twice with FACS Buffer and run on the Cytoflex LX Flow Cytometer (Beckman Coulter) for analysis. Antibody response was quantified by calculating the fold change of the geometric mean fluorescent intensity of a mouse in an experimental group over naïve control mice.

### Evaluation of Cellular Specificity

Intracellular staining of IFNγ using flow cytometry was used to show specificity of CTLs. CTLs were harvested on day 14 of the expansion culture and re-stimulated with 1×10⁵ tumor cells at a ratio of 10:1. In each experiment, at least one non-target tumor cell line from the same mouse background was used as a control. After 24 hours of re-stimulation cells were harvested, stained for flow cytometry as described above and run on the Cytoflex LX Flow Cytometer (Beckman Coulter) for analysis. Kaluza software (version 2.1, Beckman Coulter) was used to calculate the proportion of IFNγ+ cells as part of the CD3+, CD4+ and CD8+ cells.

Specific cytotoxicity of CTLs was measured as described in Evaluation of cellular immune response with the only difference that effector-target-cell ratios of 1:1, 3:1, 10:1 and 30:1 were tested depending on the tumor model. B16F10-luc2 or KP938.3-luc2 and H441-luc2, K1975-luc2 or JimT1-luc2 target cells were used for evaluation of specific cytotoxicity of murine or human CTLs, respectively. For the measurement of specific cytotoxicity of human CTLs only, an excess (10-fold more than target cells) of non-luciferase-expressing K562 cells were added to each well to reduce the measurement of unspecific components of cytotoxicity and unmask the specific CTL response.

### Synergy of Adoptive Cellular Components In Vitro

Cytotoxicity experiments were performed as described in Evaluation of cellular immune response at effector-target-cell ratios of 0.3:1, 1:1, 3:1, 10:1 and 30:1. Effector cells used were either only LAKs, CIKs, γδT-cells or CTLs, the combined cell therapy product containing all 4 components or all subgroups consisting of 2 or 3 components. When used in combination, adoptive cellular therapy components were always used in equal measures. Synergy was calculated by adopting the Chou-Talalay⁷⁷ method developed for calculating the synergy of drug combinations. Calculations were performed with the CompuSyn (version 1.0) software⁷⁸. For each combination of adoptive cellular therapy components, the combination index was calculated at the median effective dose (ED50) and compared.

### Blocking Experiments

Blocking experiments were performed as described in Evaluation of cellular immune response at effector-target-cell ratios of 10:1. B16F10-luc2 or KP938.3-luc2 and H441-luc2, K1975-luc2 or JimT1-luc2 target cells were used for the evaluation of cytotoxicity of effector cells with and without blocking antibodies. For blocking recognition via class I, class II and NK 1.1 by murine effector cells, antibodies against murine H-2 (clone M1/42.3.9.8, BioXCell), murine I-A (clone Y-3P, BioXCell) and NK1.1 (PK136, BioXCell) were used, respectively. For blocking recognition via class I, class II and NKG2D by human effector cells, antibodies against human HLA-A, -B and -C (clone W6/32, Biolegend), human HLA-DR, -DP, -DQ (clone T039, Biolegend) and NKG2D (clone 1D11, Biolegend) were used, respectively. All blocking antibodies were added to wells containing target cells at a concentration of 5 μg/ml before addition of effector cells to target cells. Relative cytotoxicity was calculated with the following formula:

$$\frac{{Cytotoxicity}_{with blocking}}{{Cytotoxicity}_{without blocking}} - 1$$

Evaluation of Cytotoxicity of aPD1 in Combination with Adoptive Cellular Therapy In Vitro

Cytotoxicity experiments were performed as described in in Evaluation of cellular immune response at effector-target-cell ratios of 1:1, 3:1, 10:1 and 30:1 for human target cells and 1:1, 3:1 and 10:1 for murine target cells. Antibodies against murine (clone RMP1-14, BioXCell) or human PD1 (Nivolumab, Bristol-Myers-Squibb) or appropriate IgG controls (Isotype control rat IgG2a, κ (BioXCell) for murine a-PD1 and clinical-grade human IgG (Octagam) for Nivolumab) were added to the wells at a concentration of 20 μg/ml before addition of effector cells to target cells.

### Next-Generation Sequencing

Samples were analyzed with the QIAseq targeted DNA panel for human lung cancer (NGHS-005X-96) or with the GeneRead DNAseq Panel PCR Kit V2 (Qiagen, Hilden, Germany). Libraries were prepared using the Gene Read DNA Library I Core Kit and the Gene Read DNA I Amp Kit (Qiagen) according to manufactures protocol. Barcoded libraries were amplified, final library products were quantified, diluted and pooled in equal amounts. Finally, 1.2 pM of the final libraries were sequenced on a NextSeq Sequencer (Illumina, San Diego, CA, USA) with the NextSeq 500 Mid Output Kit v2 following manufacturer's recommendations. The following regions were analyzed: ALK (Exon 2-15, 17-29), ATM (Exon 2-13, 15-45, 47-63), BRAF (Exon 2-4, 6-18), EGFR (Exon 2-26, 28), ERBB2 (Exon 1, 3, 5-6, 8-12, 14-26), ERBB4 (Exon 1-6, 8-13, 15-28), FGFR1 (Exon 2, 4-10, 12-18), FGFR2 (Exon 2-18), KDR (Exon 2-20, 22-30), KEAP1 (Exon 3-6), KIT (Exon 2-21), KRAS (Exon 2-6), MET (Exon 2-21), NFE2L2 (Exon 2-5), PDGFRA (Exon 2-23), PIK3CA (Exon 2-10, 12-16, 18-21), PIK3CG (Exon 3-10), RET (Exon 2-10, 12-20), ROS1 (Exon 1-43), SMARCA4 (Exon 2-5, 7-17, 19-27, 29, 30, 32-35), STK11 (Exon 1-2, 4-8), TP53 (Exon 2, 4-11).

### Clonality Analysis

PCR reactions for TCRB and TCRG T-Cell Clonality Assays (Invivogen, San Diego, CA) were carried out in duplicates using 25 and 50 ng DNA. The reaction conditions were set to a final volume of 25 μl with 5 μl primermix, 12.5 μl Multiplex PCR PlusKit (Qiagen) and water. Cycle conditions started with a first step at 95° C. for 5 minutes, followed by 35 cycles with 95° C. for 30 seconds, 60° C. for 90 seconds and 72° C. for seconds, prolonged extension at 68° C. for 30 minutes and hold at 8° C. Depending on amplification results, samples were diluted and 12 μl of Hi-Di formamid (Applied Biosystems) and 0.25 GeneScan LIZ standard was added. Samples were denatured at 95° C. for 2 minutes, cooled down at 4° C. for 5 minutes and loaded on the AB13500. PCR products were analysed by GeneScan profiling according to the Euroclonality recommendations (http://www.euroclonality.org/).

### RNASeq

RNA was extracted from fresh frozen (FF) tissue with the RNeasy Mini Kit (Qiagen) and from FFPE tissue with the RSC RNA FFPE Kit (Maxwell, San Diego, CA). Both where used according to manufacturer's instructions. Only FF or FFPE samples were compared among each other. 3′mRNA libraries were generated from total RNA using the Lexogen QuantSeq kit according to the standard protocol. After validation (2200 TapeStation, Agilent Technologies, Santa Clara, CA) and quantification (Qubit System, Invitrogen, Carlsbad, California) pools of cDNA libraries were generated. The pools were quantified using the KAPA Library Quantification kit (Peqlab, Radnor, PA) and the 7900HT Sequence Detection System (Applied Biosystems, Foster City, PA) and subsequently sequenced on an Illumina HiSeq4000 or NovaSeq6000 sequencer using a 1×50 bp protocol.

FASTQ files of 3′ UTR RNA-sequencing were checked for quality using FastQC (version 0.11.4)⁷⁹ and reads mapped to the human reference genome GRCh38.95 or the mouse reference genome GRCm38 (p6), respectively using the STAR aligner (version 2.7.0)⁸⁰. Expression was quantified prior to downstream analysis with RSEM (version 1.3.1)⁸¹. Analyses were run on the computing cluster of the Regional Computing Center of the University of Cologne (RRZK).

Gene expression of specific genes was compared by comparing z-scores of counts per million (CPM). To analyze transcripts together that are linked to the same biological process, we defined an immunogenic cell death (CALR and HMGB1)⁸² and M1/M2 macrophage polarization signatures⁸³. For the M1/M2 macrophage polarization signature we calculated a differential z-score adding all M1 polarization signature gene z-scores and subtracting all M2 polarization gene z-scores.

### Inference of Tumor Immune Microenvironment Based on Differential Gene Expression

To infer the composition of the tumor immune microenvironment based on gene expression, we created a curated list of immune cell subtype specific transcripts. We started with a list of immune cell subtype enriched genes as defined in the Nanostring Vantage 3D RNA:Protein Immune Cell Profiling Assay (Nanostring) and simplified it by omitting transcripts that are enriched in multiple immune cell subtypes, only keeping transcripts that are unique to a specific immune cell subtype. For each experiment z-scores of CPM were calculated for all transcripts. Z-scores for all samples in a group and all transcripts unique to a specific immune cell subtype were compared between groups to identify differences in the cellular composition of the tumor immune microenvironment.

### Gene Set Enrichment Analysis

Gene Set Enrichment Analysis (GSEA) was performed using GSEA software (version 4.0.2, Broad Institute). Z-scores of CPM were used as input. Our aim was to focus on gene sets relevant for certain aspects of immune function in the context of cancer. Therefore, we curated gene sets based on the nCounter Mouse PanCancer Immune Profiling Panel (Nanostring). Analyses were run with 1000 permutations, excluding only gene sets smaller than 5 genes but otherwise with standard settings.

### TCGA Analysis of Outcome by Diversity of Immune Cell Infiltration

CIBERSORT²⁰ immune cell fractions of TCGA samples were obtained from a pre-calculated published dataset⁸⁴. Using R⁶⁴, we plotted Kaplan-Meier overall survival curves for all patients, adjusted for cancer type, the lung cancer (LUAD+LUSC), the melanoma and the sarcoma cohort for patients above or below the whole-cohort median number of T-cells, NK cells and γδT-cells, respectively. While the fractions of γδT-cells were used as provided in the dataset, T-cells were defined as the sum of the CD4+ memory activated, CD4+ memory resting, CD4+ naïve and CD8+ cell fractions for this analysis. NK cells were defined as the sum of the NK cell activated and NK cell resting fractions. To estimate diversity of immune cell infiltration, TRIScore was calculated and defined as a score from 0-3 with one point given for an above whole-cohort median infiltration by T-cells, NK cells and γδT-cells, respectively

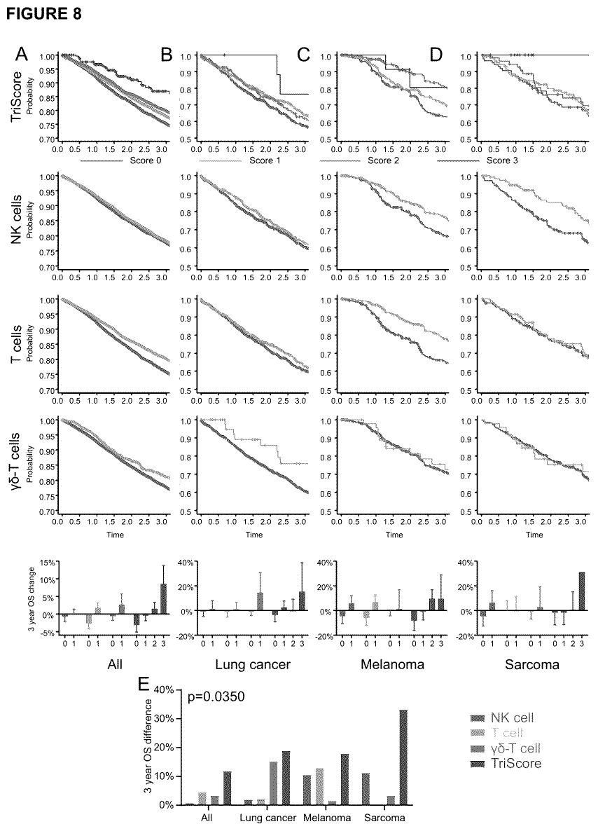

EXAMPLE 1: Combined adoptive cellular therapy displays synergistic anti-tumor activity. To investigate whether the presence of diverse effector cells in the TIME is associated with improved patient outcome, CIBERSORT was used on TCGA data and to construct a straightforward heuristic score with one point given for above median infiltration of T-cells excluding γδ-T-cells, NK-cells and γδ-T-cells. CIBERSORT is a well-validated tool to deconvolute immune cell abundances in tumors²⁰. It was found that combined tumor infiltration of these cells, as measured by our score, is a better predictor of prolonged overall survival than infiltration by any of the score components (FIG. 8A-E) in an all-cancer cohort (FIG. 8A) as well as in entity-specific subgroups, such as lung cancer (FIG. 8B), melanoma (FIG. 8C), and sarcoma (FIG. 8D). Thus, it was hypothesized that a combination of adoptive cellular therapy (ACT) components representing these innate and adaptive effector cells might display synergistic efficacy against tumor cells. Specifically, three innate-acting, functionally diverse, largely major histocompatibility complex (MHC)-unrestricted effector cell types with partly distinct recognition mechanisms were combined, lymphokine-activated killer cells (LAKs, i.e. IL-2 stimulated PBMCs)²¹, cytokine-induced killer cells (CIKs, i.e. IFNγ-pretreated, anti-CD3 and IL-2 stimulated PBMCs)²² and Vγ9Vδ2-T-cells (γδ-T-cells, i.e. PBMCs selectively stimulated with zoledronic acid and IL-2)²³ with tumor-specific, MHC-restricted cytotoxic T-lymphocytes (CTLs)^{24,25}. Of note, tumor specific CTLs were generated by a recently published co-culture method²⁴ which did not require prior knowledge of any tumor-associated or neoantigen as for example CAR-T-cells do. Thus, CTLs were tumor-specific, yet antigen-agnostic.

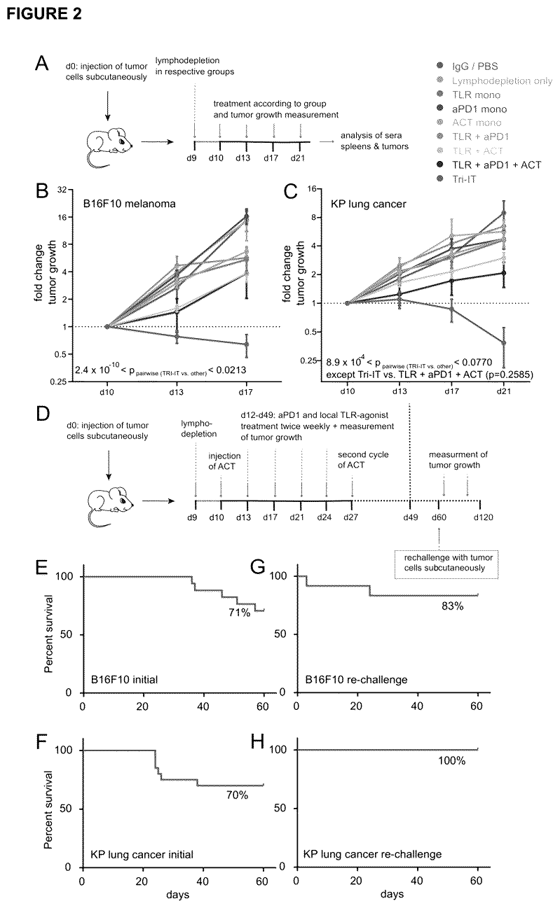

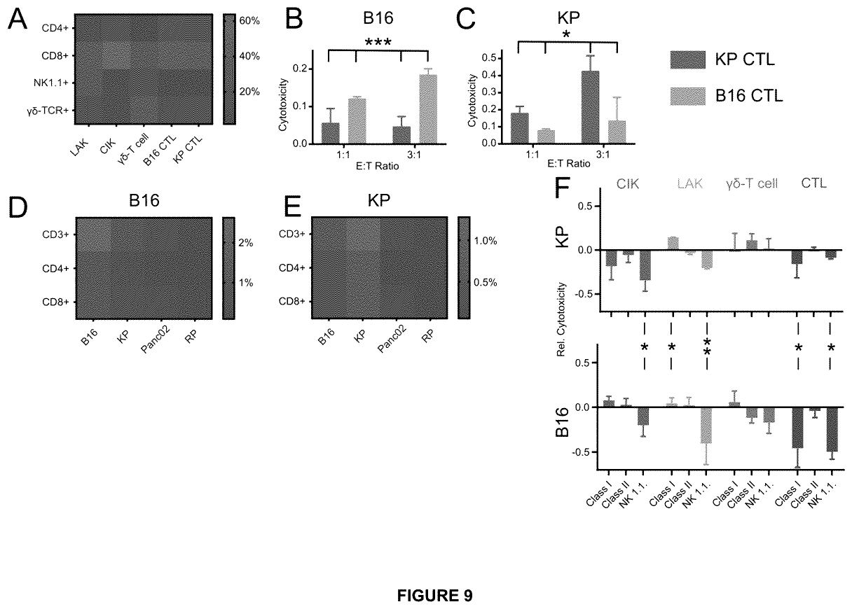

First, it was tested whether combined ACT of these innate and adaptive effector cells is more efficacious than single cell type ACT using in vitro toxicity assays in poorly immunogenic B16F10 melanoma and Kras^{LSL.GL12D/wt}; Tp53^{fl/fl}-derived (KP) lung cancer models. Of note, these models are known to be poorly immunogenic despite the fact that melanoma and lung cancer are often quite immunogenic in humans²⁶. Murine equivalents of these effector cells, which showed the expected phenotype (FIG. 9A), specificity (FIG. 9B-E) and functionality (FIG. 9F) were expanded. These experiments also further supported the rationale of combining the four selected ACT components beyond synergy, as each ACT component has a unique phenotype and mechanism of resistance (FIG. 2A,F). For example, tumors would resist CTL attack when they lose antigen presentation via MHC class I, while such an immune escape by the tumor might at the same time increase their susceptibility to LAKs.

Increasing synergy was found in higher-order combinations with the quadruple combination showing maximum synergy as evidenced by a low combination index (FIG. 1A). Dual and triple sub-combinations consisting of both innate-acting (e.g. LAKs or CIKs) and tumor-specific effector cells (e.g. CTLs) were more synergistic than innate-only combinations (FIG. 1A). To substantiate these findings in vivo, the synergy of the quadruple combination of LAKs, CIKs, γδ-T-cells and CTLs (Combined ACT) and each sub-component at the same cell dose was tested—i.e., either 1×10⁷ cells of a subcomponent or 2.5×10⁶ cells of each subcomponent for a total of also 1×10⁷ cells—in the B16F10 model (FIG. 1B). Combined ACT had a stronger inhibitory effect on tumor growth than each ACT subcomponent (FIG. 1C). To disentangle the effect of combined ACT on the TIME composition, immune cell deconvolution based on 3′mRNAseq of bulk tumor tissue harvested at the end of the experiment was applied. A higher infiltration of macrophages, Th1, Th2 and Th17 polarized T-cells, Tfh cells, cytotoxic T-cells and γδ-T-cells into tumors in combined ACT compared to single ACT treated mice was found (FIG. 1D).

Next, the functional state of the TIME by quantifying intra-tumoral transcripts of common chemo- and cytokines was analyzed. Higher levels of Eotaxin, M-CSF, LIF, MIP-1a, MIP-1b, IP-10 and MIG in combined ACT compared to single ACT treated mice were detected, indicating a broad increase in important anti-tumor chemo-attractants (FIG. 1E).

The circulatory cytokine signature was rather similar in all single ACT treated mice, with the exception that IL1b, IL-10 and LIX was detected at higher levels in LAK treated mice and MIP-2 at higher levels in CTL treated mice. In contrast, TNFα, IFNγ, IL7, IL15, IL17, IP10 and M-CSF were significantly increased in the circulation of combined ACT (FIG. 1F-G). Together, these data indicate that combined ACT might lead to a more Th1-polarized immune response with high levels of homeostatic cytokines (e.g. IL7, IL15) associated with increased NK and T-cell activity, proliferation and survival^{27,28}. In contrast, efficacy of single ACT might be limited by high levels of immunosuppressive cytokines (e.g. IL10)²⁹ or LIX, which has recently been described as tumor growth and metastasis promoting^{30,31}.

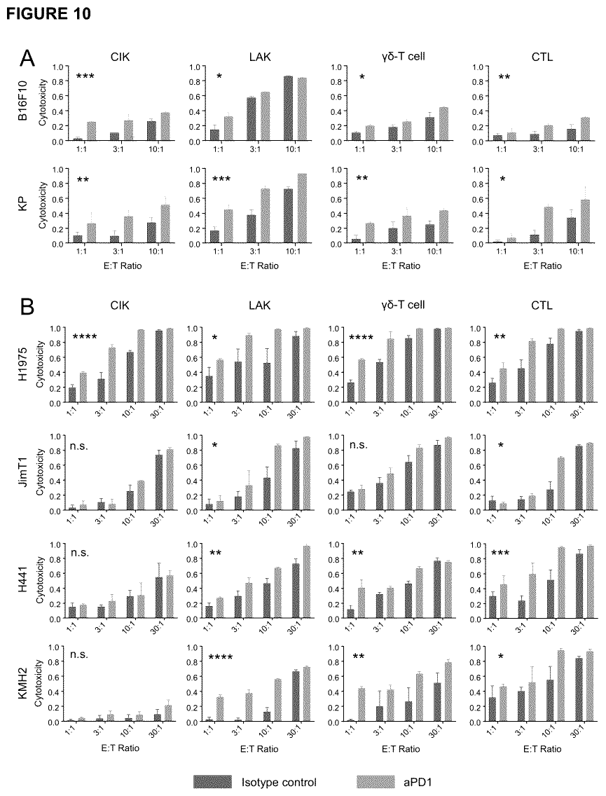

Next, it was tested if an aPD1 antibody augments the cytotoxicity of ACT subcomponents. As expected, it was found that addition of aPD1 significantly increased cytotoxicity of LAKs, CIKs, γδ-T-cells and CTLs (FIG. 10A) targeting B16F10 melanoma and KP lung cancer in vitro. These results were largely confirmed in human lung- and breast cancer and lymphoma models (FIG. 10B).

To corroborate the findings, the synergy of combined ACT was validated in an alternative, allogeneic human context (see below for validation of allo-specificity of human ACTs). In vitro synergy of the combined ACT regimen in JimT1 human breast cancer, H441 and H1975 human lung cancer and L428, L540, L1236 and KMH2 human lymphoma was found. Again, synergy was mainly observed in combinations of innate-acting and tumor-(allo)-specific effectors (FIG. 1H). Finally, combined human ACT in a humanized, H1975 lung cancer xenograft model in NSG mice was tested, also observing synergy of combined ACT (FIG. 1I).

Altogether, these data show that combined ACT of equal numbers of LAKs, CIKs, γδ-T-cells and tumor-specific CTLs is superior to subcomponent single cell type ACT in vitro and in vivo in multiple models and associated with beneficial changes in the TIME. While recognizing the complexity of the quadruple combined ACT, it was decided to use it in the further development of TRI-IT because the aim was a universal combination immunotherapy approach and dual or triple sub-combinations were not maximally effective in at least one tested in-vitro model (FIG. 1A, H). Furthermore, blockade of the aPD1 axis significantly increases cytotoxicity of ACT.

EXAMPLE 2: TRI-IT eradicates established, poorly immunogenic tumors and induces durable anti-tumor immunity. It was next investigated whether additional activation of TLRs enhances immune cell response, improves immune cell recruitment to the tumor site and, thus, improves efficacy of ACT and aPD1 treatment. For these experiments, the B16F10 melanoma and KP lung cancer model (FIG. 2A) were used, both models are known to be refractory to conventional immunotherapeutic approaches^{32,33}. Consistent with previous data, treatment of established (˜50-100 mm³) B16F10 melanomas with single-strategy immunotherapy (aPD1, TLR agonist mix, combined ACT) resulted in only marginally better tumor control compared to IgG/PBS controls (FIG. 2B). Dual combinations provided only slightly better tumor control then single-strategy immunotherapy, as did the triple combination of aPD1, TLR agonist mix and combined ACT without lymphodepletion. In contrast, triple combination of aPD1, combined ACT with TLR agonist mix including lymphodepletion—the full TRI-IT protocol—led to massive tumor shrinkage in both models (FIG. 2B-C).

Motivated by this finding, long-term tumor control was investigated after TRI-IT and treated mice with up to 2 cycles of TRI-IT with survival as the primary endpoint (FIG. 2D). Strikingly, 71% of animals in the B16F10 (FIG. 2E) and 70% of animals in the KP model (FIG. 2F) achieved complete tumor rejection after 60 days.

To assess durable anti-tumor immunity, these mice were re-challenged on day 60 without further treatment. Remarkably, 83% of B16F10 melanoma (FIG. 2G) and 100% of KP lung cancer bearing animals (FIG. 2H) were immune to re-challenge showing no tumor growth.

To assess if local TLR agonist mix treatment is only active locally, injected tumors were compared with non-injected tumors in all groups where the TLR agonist mix was part of the treatment. No difference was observed between injected and non-injected tumor growth in both models (FIG. S4C-D), indicating that local immunotherapy with the TLR agonist mix likely induces systemic, abscopal effects, although at least partial distribution of locally injected TLR agonist to the contralateral tumor site cannot be ruled out. Taken together, TRI-IT is a highly effective, antigen-agnostic, combination immunotherapy regimen that can cure established, poorly immunogenic tumors and induce durable anti-tumor immunity.

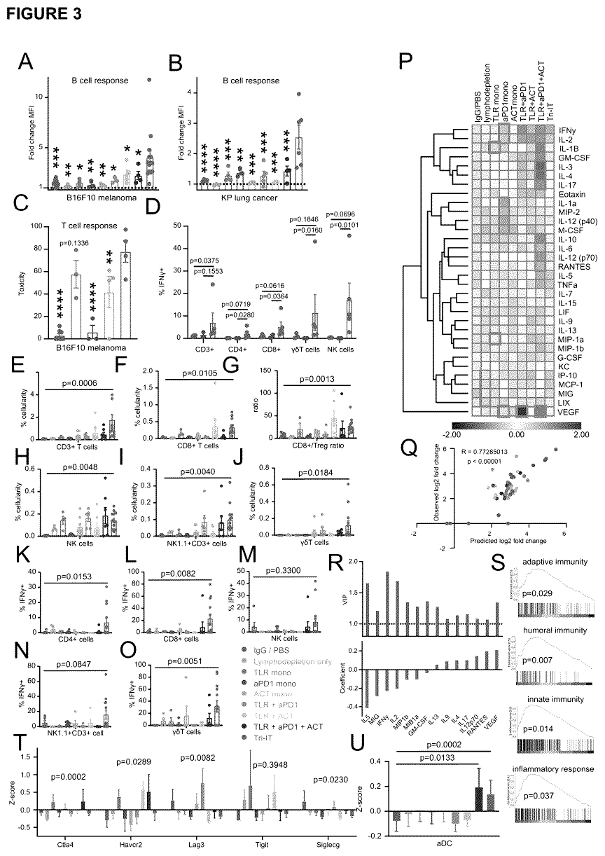

EXAMPLE 3: TRI-IT orchestrates a broad anti-tumor immune response in poorly immunogenic tumors. Motivated by the high efficacy of TRI-IT in two distinctively different, poorly immunogenic tumor models, the aim was to unravel the cellular and molecular mechanisms of TRI-IT. To quantify humoral anti-tumor immunity, circulating anti-tumor antibodies were measured. TRI-IT led to high-titer anti-tumor antibody responses that were higher than in all other groups in both models (FIG. 3A-B). To quantify cellular anti-tumor immunity of B16F10-bearing mice, cytotoxicity and intracellular IFNγ expression were measured in various splenocyte subsets upon co-incubation with B16F10 targets. Compared to IgG/PBS and aPD1 treated mice, splenocytes of TLR agonist, combined ACT and TRI-IT-treated mice exhibited higher cytotoxicity (FIG. 3C). Compared to IgG/PBS and aPD1 treated mice, a significantly higher proportion of CD4+ and CD8+ T-cells, γδ-T-cells and NK-cells from spleens of TRI-IT-treated mice were IFNγ+ after co-incubation (FIG. 3D). Together, both assays demonstrate that Tri-IT induces tumor-specific cellular immunity.

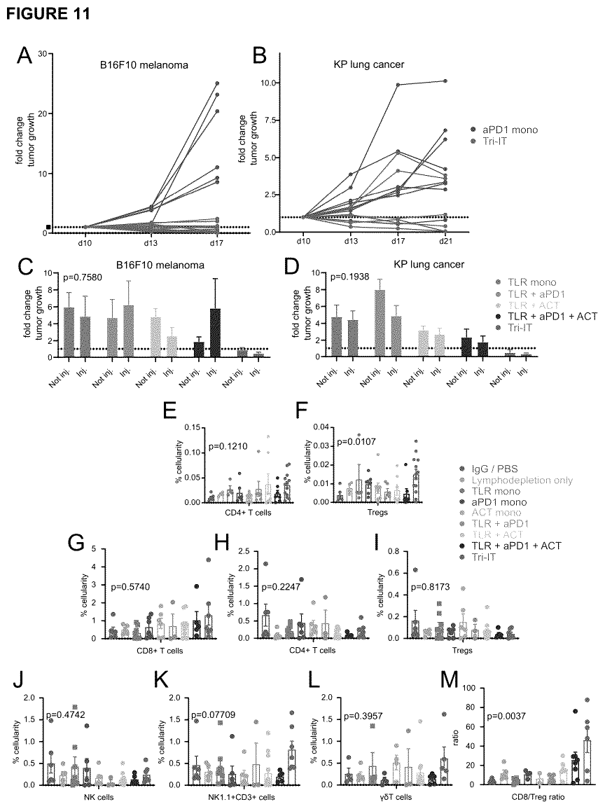

Next, the composition of the B16F10 melanoma TIME was studied in different groups by flow cytometry. A pattern of increased infiltration of tumors by T-cells (FIG. 3E), and more specifically CD8+ T-cells (FIG. 3F) with a trend towards increased infiltration by CD4+ T-cells (FIG. 11D), a higher CD8+ T-cell/Treg-ratio (FIG. 3G), and increased infiltration by NK- and NK1.1+CD3+ cells (FIG. 3H-I) was observed in higher order sub-combinations. Treg infiltration was similar in all groups (FIG. 11F). Only TRI-IT led to a high γδ-T-cell infiltration (FIG. 3J).

As IFNγ+ cells in the TIME reflect local anti-tumor immunity better than immune cell quantities³⁴, the proportion of IFNγ+ cells of tumor-infiltrating CD4+ (FIG. 3K) and CD8+ T-cells (FIG. 3L), γδ-T-cells (FIG. 3M), NK-cells (FIG. 3N) and NK1.1+CD3+ cells (FIG. 3O) was measured by flow cytometry. Only TRI-IT increased the intra-tumoral proportion of IFNγ+ cells across all immune cell subsets (FIG. 3 K-O).

In KP lung cancer, differences in TIME composition between groups were not as pronounced compared to B16F10 melanoma, though showed similar trends (FIG. 11G-M). To further elucidate the shape of the systemic immune response circulatory cyto- and chemokines were measured. Lymphodepletion led to increased systemic levels of IP-10, MCP-1, MIG and LIX. Local treatment with TLR agonist mix led to increased levels of IL-1b and MIP-1a, while aPD1 treatment led to increased levels of IL-2 and IFNγ, as well as IL-1a, MIP-2, IL-12(p40), M-CSF and VEGF. These signatures where largely maintained when treatments were combined. However, we also found increased systemic levels of cytokines such as IL-3, IL-4, IL-17, IL-10, IL-6, IL-12(p70) and RANTES (FIG. 3P) in higher order combinations. Acknowledging the complex spatial and temporal context-dependency of cytokines³⁵, the observed patterns could point to a possible rebalancing of host immunity towards Th2-polarization (IL-4, IL-6, IL-10)³⁶, myeloid-derived suppressor cell support (IL-17)³⁷ and M2-polarization of macrophages (IL4, IL-10)^{38,39} upon combination immunotherapy without lymphodepletion. This signature was diminished with the full TRI-IT protocol including lymphodepletion while the likely beneficial, pro-inflammatory, Th1-polarizing signature elicited by TRI-IT subcomponents, was largely maintained.

To further understand the contribution of single cytokines to anti-tumor response with TRI-IT, a partial least squares regression model was constructed using peripheral cytokine concentrations as input predicting tumor size across treatment groups with high accuracy (FIG. 3Q). Cytokines associated with smaller tumors were IL-5, MIG, IFNγ, IL-2, MIP-1a and MIP-1b. Cytokines associated with increased tumor growth were IL-4, IL-17, IL12p(70), RANTES and VEGF (FIG. 3R). Interestingly, this pattern of cytokines associated with larger tumors had significant overlap with those that were suppressed in the full TRI-IT protocol by the addition of lymphodepletion (FIG. 3P). Gene set enrichment analysis was used to investigate signatures of immune response, revealing significant enrichment of an adaptive, humoral, innate immune and inflammatory response signature in TRI-IT-treated mice (FIG. 3S).

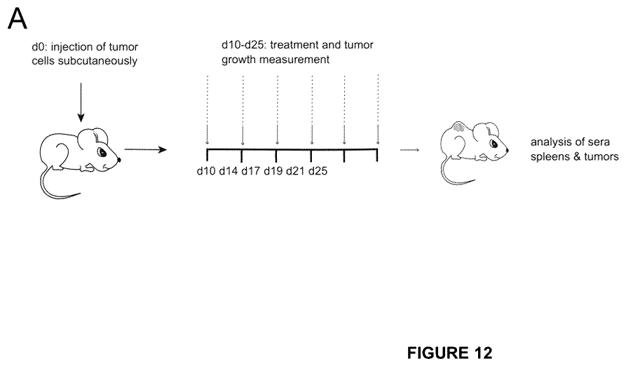

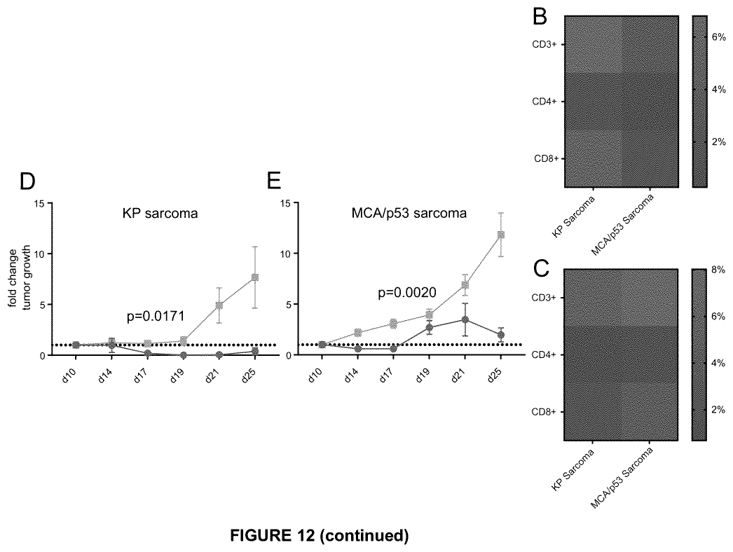

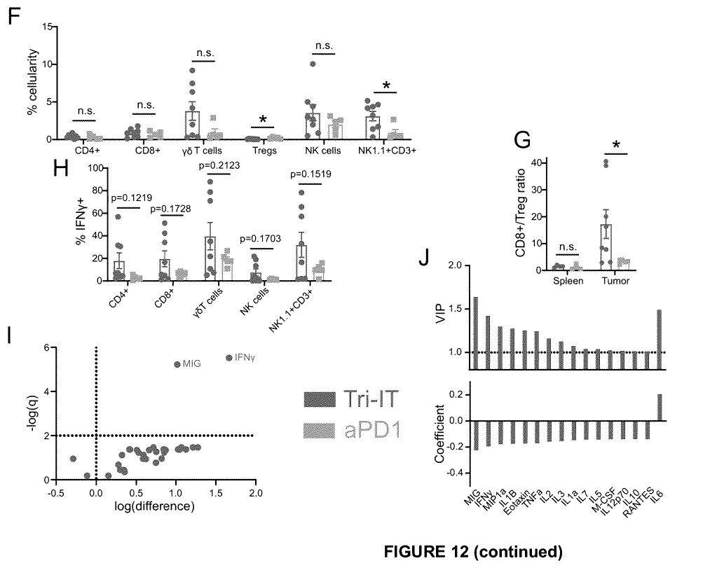

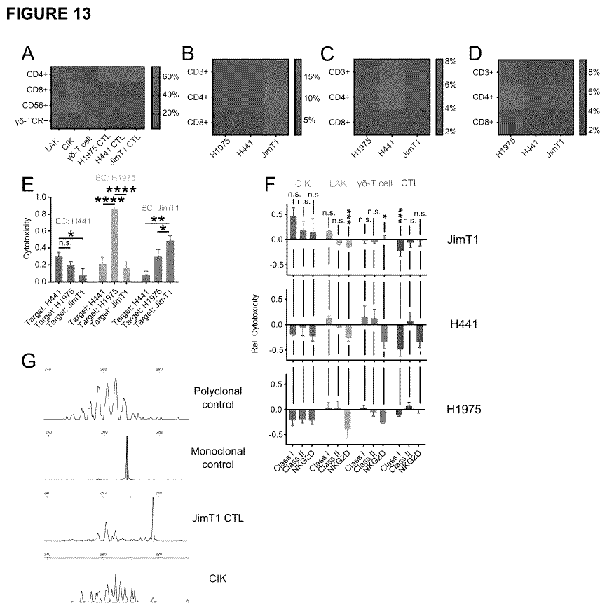

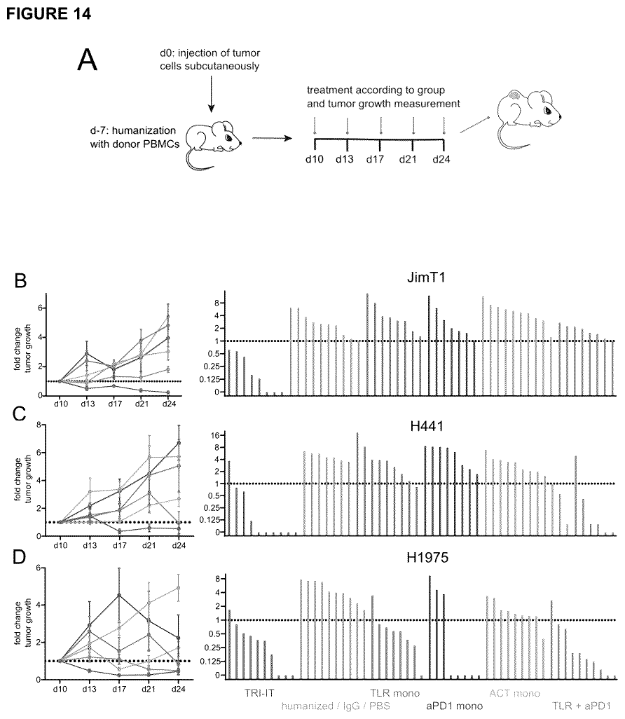

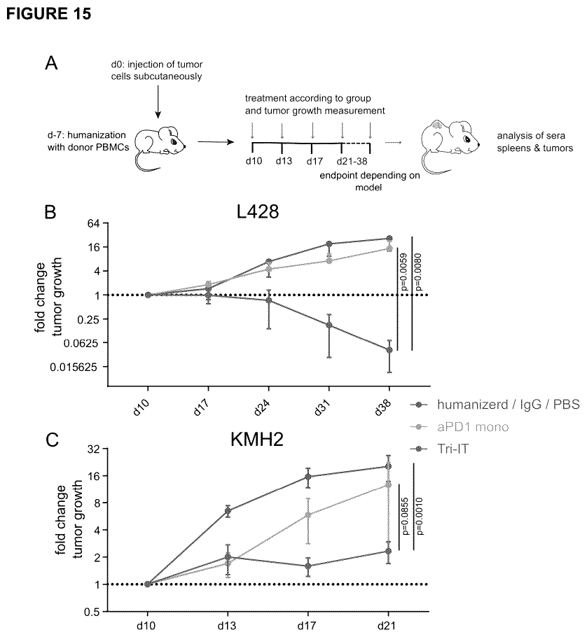

Upregulation of alternative checkpoints has been recognized as an immunotherapy resistance mechanism⁴⁰. Therefore, intra-tumoral gene expression of CTLA4, HAVCR2, LAG3, TIGIT and SIGLECG was compared by 3′mRNAseq of bulk tumor tissue across groups. Interestingly, lower-order sub-combinations of TRI-IT treatment elements led to varying upregulation of one or multiple of these alternative checkpoints, which was abrogated by TRI-IT (FIG. 3T). Presence of activated dendritic cells is crucial for a tumor-specific, adaptive immune response⁴¹. Quantified by immune cell deconvolution, tumors from mice treated with TRI-IT or the TLR+aPD1+ACT combination without lymphodepletion contained more activated dendritic cells than other groups (FIG. 3U). Altogether, above experiments confirm that TRI-IT induces adaptive, humoral and cellular immune responses that are accompanied by a broadly increased infiltration of adaptive and innate effector cells into tumors and a systemic cytokine response characterized by an increase in inflammatory, Th1-response-associated cytokines and a decrease in immunosuppressive cytokines. As soft tissue sarcomas are known to be refractory to immune checkpoint inhibitors, it was sought to evaluate TRI-ITs efficacy in two separate models of murine KP-derived and chemically (methylcholanthrene, MCA)-induced MCA/p53 sarcoma42 (FIG. 12A). First, tumor-specificity of CTLs (FIG. 12B-C) was confirmed. In both sarcoma mouse models, high efficacy of TRI-IT was observed when treating established tumors (FIG. 12D-E). Tumors from TRI-IT-treated mice exhibited higher infiltration of NK1.1+CD3+ cells, a trend towards higher infiltration of γδ-T and NK-cells and a lower infiltration of Tregs (FIG. 12F). Tumors from TRI-IT-treated mice had a higher intra-tumoral CD8+/Treg-ratio (FIG. 12G) and a trend towards an increase in intra-tumoral IFNγ+ cells among CD4+ and CD8+ T-cells, γδ-T-cells, NK cells and NK1.1+CD3+ cells (FIG. 12H). Among circulating cytokines, IFNγ and MIG were significantly increased in TRI-IT-treated mice (FIG. 12I) and associated with tumor response (FIG. 12J). Overall, the observed immunological changes in the independent sarcoma models closely matched those in the KP lung and B16F10 models, pointing to universal and not model-specific changes induced by TRI-IT. Additionally, the efficacy of TRI-IT was investigated in different allogeneic humanized mouse models of lymphoma, non-small-cell-lung-cancer (NSCLC) and breast cancer demonstrating its efficacy in different lymphoma and oncogene-driven solid tumors (FIG. 13A-G, FIG. 14A-D and FIG. 15A-C).

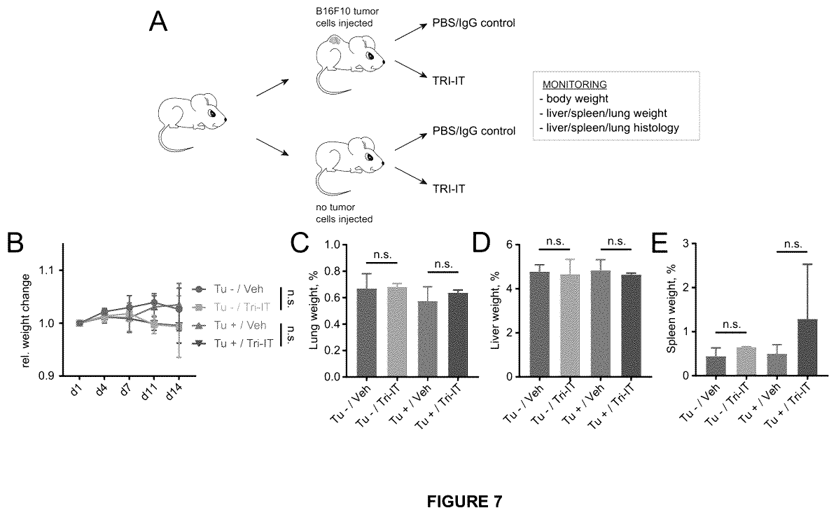

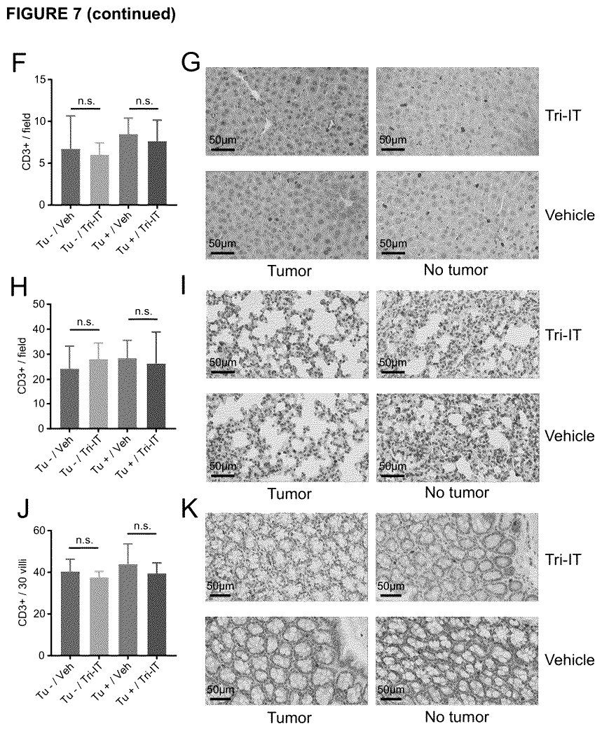

EXAMPLE 4: TRI-IT is not associated with off-target toxicity. Combination immunotherapy has been associated with profound toxicity⁴³. Therefore, it was sought to evaluate common toxicities associated with combination immunotherapy in mice. Weight loss, organ weight and CD3+ T-cell infiltration into organs were compared as surrogates for off-target immune-mediated toxicity between TRI-IT and control treated animals with or without subcutaneous B16F10 melanomas (FIG. 7A). Reassuringly, only a minimal, non-significant (≈3%) difference in weight change was found between TRI-IT and IgG/PBS treated animals (FIG. 7B) and no difference in lung weight (FIG. 7C), liver weight (FIG. 7D), spleen weight (FIG. 7E) and CD3+ T-cell infiltration into liver (FIG. 7F-G), lung (FIG. 7H-l) and colon (FIG. 7J-K). Altogether, these results indicate that TRI-IT is safe and has low off-target immune-mediated toxicity.

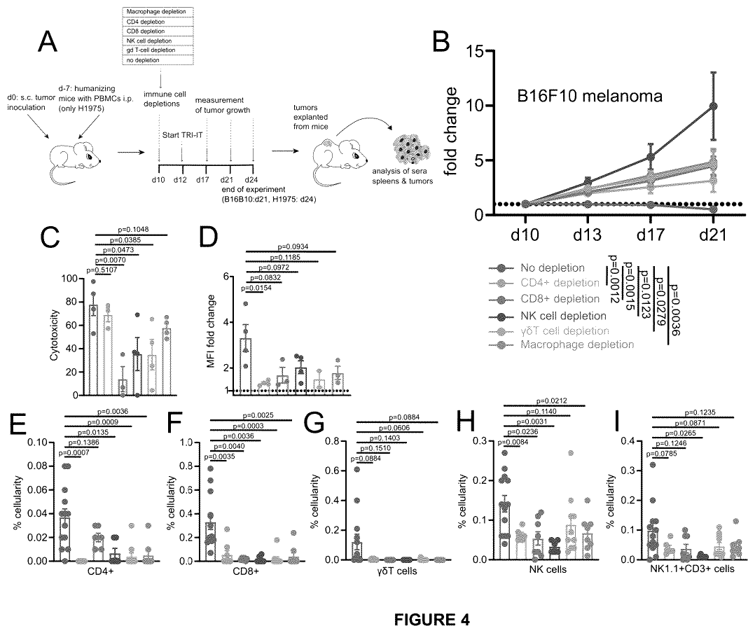

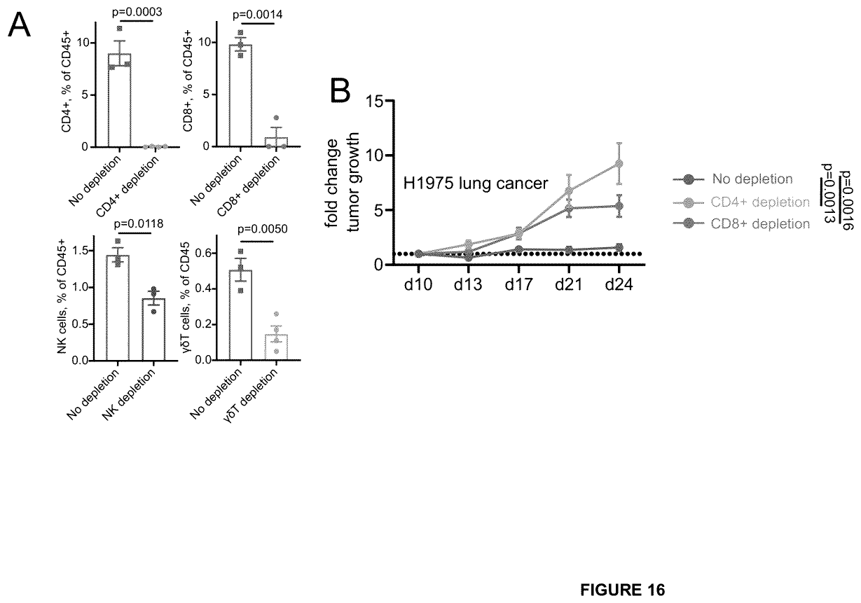

EXAMPLE 5: Depletions of CD4+ and CD8+ T-cells, NK− cells, γδ-T-cells and macrophages reduce efficacy of TRI-IT. Next, it was sought to elucidate the contribution of immune cell subsets to the efficacy of TRI-IT by depleting them in TRI-IT-treated, B16F10-bearing mice (FIG. 4A). Depletions were confirmed by flow cytometry (FIG. 16A). Surprisingly, depletions of either CD4+ or CD8+ T-cells, NK-cells, γδ-T-cells or macrophages all decreased the therapeutic effect of TRI-IT (FIG. 4B). To unravel the mechanism of the diminished efficacy of TRI-IT by the depletions, cellular and humoral anti-tumor immunity were quantified. Depletions of CD8+ T-cells, NK-cells and γδ-T-cells, all key mediators of adaptive or innate cellular immunity, led to reduced cellular anti-tumor immunity (FIG. 4C). Depletion of CD4+ T-cells, which are crucial for the induction of humoral immunity⁴⁴, and γδ-T-cells led to the biggest reduction in humoral anti-tumor immunity (FIG. 4D). Interestingly, in addition to absence of the depleted immune cell subtype, depletion of any immune cell subtype also led to a broadly reduced immune cell infiltration of tumors in general (FIG. 4E-I), suggesting that TRI-IT induced TIME changes depend on the presence of a broad spectrum of immune cells. Further, it was sought to confirm these findings in the humanized allogeneic H1975 lung cancer xenograft model. Reminiscent of the B16F10 model, depletion of CD4+ and CD8+ cells resulted in decreased efficacy of TRI-IT (FIG. S9B).

Taken together, it was demonstrated that CD4+ and CD8+ T-cells, macrophages, NK-cells and γδ-T-cells are crucial for TRI-IT's efficacy.

EXAMPLE 6: TRI-IT displays high efficacy in autologous humanized patient-derived mouse models of lung cancer. Profound differences between murine and human immunity necessitate immunotherapy models that are as close as possible to human cancer. Preclinical evaluation of novel cancer treatments in models as close as possible to actual patients is vital. To this end, TRI-IT's efficacy in autologous humanized cancer models^{45,46} was evaluated. Humanized patient-derived xenograft (PDX) models from two different NSCLC patients (FIG. 5A) were created.

First, specificity of autologous anti-PDX CTLs was confirmed, revealing high specificity of especially CD8+CTLs towards their target PDX (FIG. 5B-D). Of note, CTLs generated from the same patient's PBMCs against early (PDX1.1) and late generation (PDX1.2) PDX were not cross-reactive. Consistent with the experiments in poorly immunogenic murine NSCLC we found a strong tumor response to TRI-IT compared to standard aPD1 monotherapy in all NSCLC PDX models (FIG. 5 E-G). As expected, TRI-IT-treated mice showed increased IFNγ and trends towards increased levels of TNFα, MIP-1a and MIP-1b in circulation (FIG. 5H). In line with the findings in murine B16F10 and KP models, increased CD4+, CD8+ and γδ-T-cell infiltration into tumors were observed in TRI-IT-treated mice (FIG. 5I) and a higher proportion of functionally active, IFNγ+CD8+T−, NK− and CD56+CD3+ cells (FIG. 5J). No significant difference between TLR-agonist-injected and non-TLR-agonist-injected tumors was observed (FIG. 5K), indicating possible abscopal effects of the local TLR agonist mix also in these models.

EXAMPLE 7: TRI-IT displays high efficacy in an autochthonous, genetically engineered lung cancer model. Finally, because the immune landscapes of transplanted tumors can differ from primary tumors that co-evolve with the immune system⁴⁷, TRI-IT was evaluated in the non-immunogenic, autochthonous, genetically defined KP model of lung cancer⁴⁸ (FIG. 6A). Intratumoral injection of the TLR agonist mix was not feasible intrapulmonary, therefore two adaptions of TRI-IT were tested: injecting the TLR agonist mix subcutaneously and administering it via inhalation. A very good tumor response to TRI-IT was observed as assessed by micro-CT scan (μCT) compared to aPD1 treatment (FIG. 6B-C). TRI-IT-treated animals had less lung affected by tumor (FIG. 6D), less lesions per lung (FIG. 6E) and trended towards smaller tumors (FIG. 6F). Assessing infiltration of T-cells into autochthonous KP tumors by IHC (FIG. 6G), higher levels of T-cells in general and the CD4+ T-cell subset and a trend towards higher levels of CD8+ T-cells in TRI-IT-treated mice were found.

To evaluate changes in the TIME more comprehensively, tumors were collected at the end of the experiment and performed bulk tumor RNA-sequencing followed by immune cell deconvolution. Intratumoral inactivated and activated dendritic cells, macrophages, neutrophils, B-cells, Th1, Th2 and Tfh cells and CD56dim NK cells were increased in TRI-IT with inhaled TLR agonist mix-treated compared to aPD1-treated mice (FIG. 6H). Examination of intratumoral cyto- and chemokine transcripts revealed an increased Th1-response associated, inflammatory and chemoattractant-rich signature in TRI-IT with inhaled TLR agonist mix-treated compared to aPD1-treated mice (FIG. 6I). As in previous models (FIG. 3T), alternative immune checkpoints were not upregulated but rather downregulated by TRI-IT in autochthonous KP lung cancer (FIG. 6J). Induction of immunogenic cell death has been described as a key goal of combination immunotherapy⁴⁹. Interestingly, increased transcripts representative of an immunogenic cell death signature, point towards induction of immunogenic cell death by TRI-IT (FIG. 6K). Analysis of differential M1 and M2 polarization signatures by 3′mRNAseq revealed a shift towards M1 polarization upon TRI-IT treatment (FIG. 6L). Examining differentially expressed transcripts between TRI-IT- and aPD1-treated mice, increased transcripts of dsRNA targets (OAS2), chemokine receptors (CCR4), and genes involved in T-cell and neutrophil activation (PIK3CD), innate immune responses to TLR activation (IRAK4) and antigen presentation (TAP1) we observed. Interestingly, a reduced transcript frequency of negative regulators of anti-tumor immune responses in TRI-IT-treated mice, such as ARG2 or LAG3 and of genes involved in induction of antigen tolerance (AIRE) (FIG. 6N) was also observed. Finally, gene set enrichment analysis was used to investigate signatures of immune response. This analysis revealed a strong enrichment of gene sets representing an adaptive, humoral, innate and inflammatory response as well as increased TLR signaling (FIG. 6O). Inhaled delivery of the TLR agonist mix led to better tumor control than subcutaneous delivery (FIG. 6B-F). This was matched by higher levels of broad intratumoral immune cell infiltration (FIG. 6G-H), an even more beneficial intratumoral cyto- and chemokine profile (FIG. 6I) and a further increased immunogenic cell death signature (FIG. 6K). It was concluded that inhaled delivery of TLR agonists as part of TRI-IT is a highly feasible and effective option for the treatment of lung tumors.

In summary, the findings in autologous PDX models and the poorly immunogenic, autochthonous KP lung cancer model confirmed the efficacy of TRI-IT, with local and systemic changes induced by TRI-IT that were largely similar to those observed in the B16F10 melanoma and KP lung cancer models.

EXAMPLE 8: Validation of murine effector cells after expansion. All murine effector cells showed the expected phenotype (FIG. 9A). Additionally, CTL's tumor-specific activity against B16F10 melanoma and KP lung cancer were validated by cytotoxicity assay (FIG. 9B-C) and intracellular IFNγ staining following tumor cell re-stimulation (FIG. 9D-E). Finally, blocking experiments with antibodies against class I and class II and NK1.1. were performed, showing that cytotoxic activity of LAKs and CIKs is NK1.1. dependent, while CTL activity is class I, and NK1.1. dependent Blocking class I enhances LAK activity (FIG. 9F). Together, these experiments confirm the expected phenotype and functionality of the murine ACT subcomponents of TRI-IT.

EXAMPLE 9: TRI-IT is effective in allogeneic humanized mouse models of lung and breast cancer. Novel immunotherapy approaches might rekindle interest in allogeneic transplantation for solid tumors^{68,69}. Therefore, it was sought to test the efficacy of TRI-IT in allogeneic humanized mouse models of lung and breast cancer. First, the expected phenotype (FIG. 13A) and CTL allo-specificity (FIG. 13B-D) of allogeneic effector CTLs were confirmed by measuring intracellular IFNγ expression upon restimulation and allo-specific cytotoxicity against luciferase-transduced target cells while simultaneously blocking unspecific reactivity with K562 “cold” targets⁷⁰ (FIG. 13E). Next, blocking experiments with antibodies against Class I, Class II and NKG2D were performed. Allogenic CIK and LAK activity was blocked by the aNKG2D antibody with a tendency towards increased LAK cytotoxicity with Class I blocking. γδ-T cell cytotoxicity was blocked by the NKG2D antibody while CTL activity was strongly inhibited with Class I blocking (FIG. 13F). As a further validation of the expected phenotype of ACT subcomponents, T cell receptor clonality analysis of selected cells was performed, confirming the polyclonal nature of CIKs and exemplary the oligoclonal nature of JimT1 CTLs (FIG. 13G).

Next, TRI-IT was compared with a humanization/IgG/PBS control, single TRI-IT components and dual TLR agonist mix+aPD1 checkpoint inhibition in three allogeneic humanized mouse models of cancer (FIG. 14A). In hormone receptor negative, trastuzumab resistant JimT1 breast cancer⁷¹, only TRI-IT led to tumor control and shrinkage of established tumors (FIG. 14B). In K-ras mutated H441⁷² NSCLC TRI-IT was superior to single TRI-IT component treatments, though the dual TLR agonist mix+aPD1 checkpoint inhibition treatment also showed some tumor control (FIG. 14C). In EGFR-driven, T790M mutated H1975 NSCLC⁷³, TRI-IT was also very effective at controlling and shrinking established tumors. However, aPD1 checkpoint inhibition alone and its combination with the TLR agonist mix was equally effective (FIG. 14D).

Altogether, these results show that TRI-IT is highly effective in allogeneic humanized cancer models across cancer entities, making it a possible addition to allogeneic transplantation for solid tumors.

EXAMPLE 10: TRI-IT is highly effective against human lymphoma. Encouraged by results so far, it was sought to evaluate TRI-IT in other, non-carcinoma cancers.

To this end, another set of allogeneic humanized mouse models of Hodgkin lymphoma (HL) was utilized (FIG. 15A). HL was chosen, because it is in principle treatable with immunotherapy⁹, having high response rates to aPD1 checkpoint blockade, but not curable by it. Humanized models were chosen because murine models of HL do not exist. TRI-IT led to tumor regression of established L428 HL (FIG. 15B) and tumor stabilization of established KMH2 HL (FIG. 15C), while aPD1 checkpoint inhibition did not.

EXAMPLE 11: TRI-IT is highly effective in an autologously humanized mouse models of cancer. Patient 1 was a 67-year-old male with UICC Stage IIIA squamous cell carcinoma of the lower left lobe. Patient 2 was a 75-year-old male with UICC Stage IIIA squamous cell carcinoma of the upper left lobe. Both PDX were generated from resected tumors. Two PDX from patient 1 were used, one early (PDX 1.1, 4^{th} generation) and one late generation (PDX 1.2>6^{th} generation). PDX identity was confirmed by sequencing key driver mutations in PDX and primary tumor samples.

### REFERENCES

1. Ferlay, J. et al. Estimating the global cancer incidence and mortality in 2018: GLOBOCAN sources and methods. International Journal of Cancer 144, 1941-1953 (2019).
2. Mellman, I., Coukos, G. & Dranoff, G. Cancer immunotherapy comes of age. Nature 480, 480-489 (2011).
3. Waldman, A. D., Fritz, J. M. & Lenardo, M. J. A guide to cancer immunotherapy: from T cell basic science to clinical practice. Nature Reviews Immunology 1-18 (2020). doi:10.1038/s41577-020-0306-5
4. Topalian, S. L. et al. Safety, Activity, and Immune Correlates of Anti-PD-1 Antibody in Cancer. N. Engl. J. Med. 366, 2443-2454 (2012).
5. Moynihan, K. D. & Irvine, D. J. Roles for innate immunity in combination immunotherapies. Cancer Research 77, 5215-5221 (2017).
6. Carbone, D. P. et al. First-Line Nivolumab in Stage IV or Recurrent Non-Small-Cell Lung Cancer. N. Engl. J. Med. 376, 2415-2426 (2017).
7. Binnewies, M. et al. Understanding the tumor immune microenvironment (TIME) for effective therapy. Nat. Med. 24, 541-550 (2018).
8. Moynihan, K. D. et al. Eradication of large established tumors in mice by combination immunotherapy that engages innate and adaptive immune responses. Nat. Med. 22, 1402-1410 (2016).
9. Ansell, S. M. et al. PD-1 Blockade with Nivolumab in Relapsed or Refractory Hodgkin's Lymphoma. N. Engl. J. Med. 372, 311-319 (2015).
10. Gandhi, L. et al. Pembrolizumab plus Chemotherapy in Metastatic Non-Small-Cell Lung Cancer. N. Engl. J. Med. 378, 2078-2092 (2018).
11. Kaczanowska, S., Joseph, A. M. & Davila, E. TLR agonists: our best frenemy in cancer immunotherapy. J. Leukoc. Biol. 93, 847-863 (2013).
12. Wesch, D. et al. Direct Costimulatory Effect of TLR3 Ligand Poly(I:C) on Human γδ T Lymphocytes. J. Immunol. 176, 1348-1354 (2006).
13. Shojaei, H. et al. Toll-like receptors 3 and 7 agonists enhance tumor cell lysis by human γσ T cells. Cancer Res. 69, 8710-8717 (2009).
14. Krieg, A. M. Toll-like receptor 9 (TLR9) agonists in the treatment of cancer. Oncogene 27, 161-167 (2008).
15. Zhao, B. G., Vasilakos, J. P., Tross, D., Smirnov, D. & Klinman, D. M. Combination therapy targeting toll like receptors 7, 8 and 9 eliminates large established tumors. J. Immunother. Cancer 2, 12 (2014).
16. Amos, S. M. et al. Adoptive immunotherapy combined with intratumoral TLR agonist delivery eradicates established melanoma in mice. Cancer Immunol. Immunother. 60, 671-683 (2011).
17. Sivick, K. E. et al. Magnitude of Therapeutic STING Activation Determines CD8+ T Cell-Mediated Anti-tumor Immunity. Cell Rep. 25, 3074-3085.e5 (2018).
18. Dudley, M. E. et al. Adoptive cell transfer therapy following non-myeloablative but lymphodepleting chemotherapy for the treatment of patients with refractory metastatic melanoma. J. Clin. Oncol. 23, 2346-2357 (2005).
19. McGuirk, J. P. et al. Tisagenlecleucel in Adult Relapsed or Refractory Diffuse Large B-Cell Lymphoma. N. Engl. J. Med. 380, 45-56 (2018).
20. Newman, A. M. et al. Robust enumeration of cell subsets from tissue expression profiles. Nat. Methods 12, 453-457 (2015).
21. Mulé, J. J., Shu, S., Schwarz, S. L. & Rosenberg, S. A. Adoptive immunotherapy of esablished pulmonary metastases with LAK cells and recombinant interleukin-2. Science. 225 (4669), 1487-1489 (1984).
22. Schmidt-Wolf, I. G. H., Negrin, R. S., Kiem, H. P., Blume, K. G. & Weissman, I. L. Use of a SCID mouse/human lymphoma model to evaluate cytokine-induced killer cells with potent antitumor cell activity. J. Exp. Med. 174, 139-149 (1991).
23. Kondo, M. et al. Zoledronate facilitates large-scale ex vivo expansion of functional γδ T cells from cancer patients for use in adoptive immunotherapy. Cytotherapy 10, 842-856 (2008).
24. Dijkstra, K. K. et al. Generation of Tumor-Reactive T Cells by Co-culture of Peripheral Blood Lymphocytes and Tumor Organoids. Cell 174, 1586-1598.e12 (2018).
25. Cattaneo, C. M. et al. Tumor organoid-T-cell coculture systems. Nat. Protoc. 15, 15-39 (2020).
26. Sanmamed, M. F., Chester, C., Melero, I. & Kohrt, H. Defining the optimal murine models to investigate immune checkpoint blockers and their combination with other immunotherapies. Ann. Oncol. 27, 1190-1198 (2016).
27. Mazzucchelli, R. & Durum, S. K. Interleukin-7 receptor expression: Intelligent design. Nature Reviews Immunology 7, 144-154 (2007).
28. Santana Carrero, R. M. et al. IL-15 is a component of the inflammatory milieu in the tumor microenvironment promoting antitumor responses. Proc. Natl. Acad. Sci. U.S.A 116, 599-608 (2019).
29. Dennis, K. L., Blatner, N. R., Gounari, F. & Khazaie, K. Current status of interleukin-10 and regulatory T-cells in cancer. Current Opinion in Oncology 25, 637-645 (2013).
30. Zhao, J. et al. Tumor-derived CXCL5 promotes human colorectal cancer metastasis through activation of the ERK/Elk-1/Snail and AKT/GSK3β/β-catenin pathways. Mol. Cancer 16, 70 (2017).
31. Romero-Moreno, R. et al. The CXCL5/CXCR2 axis is sufficient to promote breast cancer colonization during bone metastasis. Nat. Commun. 10, 1-14 (2019).
32. Pfirschke, C. et al. Immunogenic Chemotherapy Sensitizes Tumors to Checkpoint Blockade Therapy. Immunity 44, 343-354 (2016).
33. Reilley, M. J. et al. TLR9 activation cooperates with T cell checkpoint blockade to regress poorly immunogenic melanoma. J. Immunother. Cancer 7, 323 (2019).
34. Magen, A. et al. Single-Cell Profiling Defines Transcriptomic Signatures Specific to Tumor-Reactive versus Virus-Responsive CD4+ T Cells. Cell Rep. 29, 3019-3032.e6 (2019).
35. Cytokines in the balance. Nature Immunology 20, 1557 (2019).
36. Kaiko, G. E., Horvat, J. C., Beagley, K. W. & Hansbro, P. M. Immunological decision-making: How does the immune system decide to mount a helper T-cell response? Immunology 123, 326-338 (2008).
37. He, D. et al. IL-17 Promotes Tumor Development through the Induction of Tumor Promoting Microenvironments at Tumor Sites and Myeloid-Derived Suppressor Cells. J. Immunol. 184, 2281-2288 (2010).
38. Biswas, S. K. & Mantovani, A. Macrophage plasticity and interaction with lymphocyte subsets: Cancer as a paradigm. Nature Immunology 11, 889-896 (2010).
39. Ohtaki, Y. et al. Stromal macrophage expressing CD204 is associated with tumor aggressiveness in lung adenocarcinoma. J. Thorac. Oncol. 5, 1507-1515 (2010).
40. Jenkins, R. W., Barbie, D. A. & Flaherty, K. T. Mechanisms of resistance to immune checkpoint inhibitors. Br. J. Cancer 118, 9-16 (2018).
41. Wculek, S. K. et al. Dendritic cells in cancer immunology and immunotherapy. Nature Reviews Immunology 20, 7-24 (2020).
42. Lee, C. L. et al. Mutational landscape in genetically engineered, carcinogen-induced, and radiation-induced mouse sarcoma. JCI Insight 4, (2019).
43. Larkin, J. et al. Combined Nivolumab and Ipilimumab or Monotherapy in Untreated Melanoma. N. Engl. J. Med. 373, 23-34 (2015).
44. Elong Ngono, A. et al. CD4+ T cells promote humoral immunity and viral control during Zika virus infection. PLOS Pathog. 15, e1007474 (2019).
45. Jespersen, H. et al. Clinical responses to adoptive T-cell transfer can be modeled in an autologous immune-humanized mouse model. Nat. Commun. 8, 1-10 (2017).
46. Choi, Y. et al. Studying cancer immunotherapy using patient-derived xenografts (PDXs) in humanized mice. Experimental and Molecular Medicine 50, 99 (2018).
47. Wisdom, A. J. et al. Single cell analysis reveals distinct immune landscapes in transplant and primary sarcomas that determine response or resistance to immunotherapy. Nat. Commun. 11, 1-14 (2020).
48. DuPage, M., Dooley, A. L. & Jacks, T. Conditional mouse lung cancer models using adenoviral or lentiviral delivery of Cre recombinase. Nat. Protoc. 4, 1064-1072 (2009).
49. Smyth, M. J., Ngiow, S. F., Ribas, A. & Teng, M. W. L. Combination cancer immunotherapies tailored to the tumour microenvironment. Nature Reviews Clinical Oncology 13, 143-158 (2016).
50. Amos, S. M. et al. Adoptive immunotherapy combined with intratumoral TLR agonist delivery eradicates established melanoma in mice. Cancer Immunol. Immunother. 60, 671-683 (2011).
51. Law, T. M. et al. Phase iii randomized trial of interleukin-2 with or without lymphokine-activated killer cells in the treatment of patients with advanced renal cell carcinoma. Cancer 76, 824-832 (1995).
52. Kakimi, K. et al. Adoptive transfer of zoledronate-expanded autologous V 39Vδ 2 T-cells in patients with treatment-refractory non-small-cell lung cancer: A multicenter, open-label, single-arm, phase 2 study. J. Immunother. Cancer 8, 1185 (2020).
53. Takayama, T. et al. Adoptive immunotherapy to lower postsurgical recurrence rates of hepatocellular carcinoma: A randomised trial. Lancet 356, 802-807 (2000).
54. Montgomery, R. B., Makary, E., Schiffman, K., Goodell, V. & Disis, M. L. Endogenous Anti-HER2 Antibodies Block HER2 Phosphorylation and Signaling through Extracellular Signal-Regulated Kinase. (2005).
55. Dieu-Nosjean, M. C. et al. Tertiary lymphoid structures, drivers of the anti-tumor responses in human cancers. Immunol. Rev. 271, 260-275 (2016).
56. Zhu, E. F. et al. Synergistic innate and adaptive immune response to combination immunotherapy with anti-tumor antigen antibodies and extended serum half-life il-2. Cancer Cell 27, 489-501 (2015).
57. Yang, X. et al. Targeting the tumor microenvironment with interferon-3 bridges innate and adaptive immune responses. Cancer Cell 25, 37-48 (2014).
58. Pfirschke, C. et al. Immunogenic Chemotherapy Sensitizes Tumors to Checkpoint Blockade Therapy. Immunity 44, 343-354 (2016).
59. Laport, G. G. et al. Adoptive immunotherapy with cytokine-induced killer cells for patients with relapsed hematologic malignancies after allogeneic hematopoietic cell transplantation. Biol. Blood Marrow Transplant. 17, 1679-87(2011).
60. Dafni, U. et al. Efficacy of adoptive therapy with tumor-infiltrating lymphocytes and recombinant interleukin-2 in advanced cutaneous melanoma: A systematic review and meta-analysis. Annals of Oncology 30, 1902-1913 (2019).
61. Frega, G. et al. Trial Watch: experimental TLR7/TLR8 agonists for oncological indications. Oncolmmunology 9, (2020).
62. Le Naour, J., Galluzzi, L., Zitvogel, L., Kroemer, G. & Vacchelli, E. Trial watch: TLR3 agonists in cancer therapy. Oncolmmunology 9, (2020).
63. Smith, M. et al. Trial Watch: Toll-like receptor agonists in cancer immunotherapy. Oncolmmunology 7, (2018).
64. The R Foundation for Statistical Computing R version 3.3.2. (https://www.r-project.org/).
65. GraphPad software. GraphPad Prism.
66. Golfmann, K. et al. Synergistic anti-angiogenic treatment effects by dual FGFR1 and VEGFR1 inhibition in FGFR1-amplified breast cancer. Oncogene 37, 5682 (2018).
67. Shaw, R., Miller, S., Curwen, J. & Dymond, M. Design, analysis and reporting of tumor models. Lab Animal 46, 207-211 (2017).
68. Llosa, N. J. et al. Reduced-Intensity Haploidentical Bone Marrow Transplantation with Post-Transplant Cyclophosphamide for Solid Tumors in Pediatric and Young Adult Patients. Biol. Blood Marrow Transplant. 23, 2127-2136 (2017).
69. Bregni, M., Badoglio, M., Pedrazzoli, P. & Lanza, F. Is allogeneic transplant for solid tumors still alive. Bone Marrow Transplantation 51, 751-752 (2016).
70. Correale, P. et al. In Vitro Generation of Human Cytotoxic T Lymphocytes Specific for Peptides Derived From Prostate-Specific Antigen.
71. Tanner, M. et al. Characterization of a novel cell line established from a patient with Herceptin-resistant breast cancer. (2004).
72. Brower, M., Carney, D. N., Oie, H. K., Gazdar, A. F. & Minna, J. D. Growth of Cell Lines and Clinical Specimens of Human Non-Small Cell Lung Cancer in a Serum-free Defined Medium. Cancer Res. 46, (1986).
73. Ghandi, M. et al. Next-generation characterization of the Cancer Cell Line Encyclopedia. Nature 569, 503-508 (2019).
74. Morpheus. Available at: https://software.broadinstitute.org/morpheus/. (Accessed: 6 Jun. 2019)
75. Benjamini, Y., Krieger, A. M. & Yekutieli, D. Adaptive linear step-up procedures that control the false discovery rate. Biometrika 93, 491-507 (2006).
76. Mevik, B. H. & Wehrens, R. The pls package: Principal component and partial least squares regression in R. J. Stat. Softw. 18, 1-23 (2007).
77. Chou, T. C. Drug combination studies and their synergy quantification using the chou-talalay method. Cancer Research 70, 440-446 (2010).
78. Chou, T. & Martin, N. CompuSyn for Drug Combinations. (2005).
79. Andreas, S. FastQC: a quality control tool for high throughput sequence data.
80. Dobin, A. et al. STAR: Ultrafast universal RNA-seq aligner. Bioinformatics 29, 15-21 (2013).
81. Li, B. & Dewey, C. N. RSEM: Accurate transcript quantification from RNA-Seq data with or without a reference genome. BMC Bioinformatics 12, 323 (2011).
82. Kepp, O. et al. Consensus guidelines for the detection of immunogenic cell death. Oncolmmunology 3, (2014).
83. Orecchioni, M., Ghosheh, Y., Pramod, A. B. & Ley, K. Macrophage polarization: Different gene signatures in M1(Lps+) vs. Classically and M2(LPS-) vs. Alternatively activated macrophages. Frontiers in Immunology 10, 1084 (2019).
84. Thorsson, V. et al. The Immune Landscape of Cancer. Immunity 48, 812-830.e14 (2018).

1. A method of preventing or treating cancer in a patient comprising administering to the patient an effective amount of a medicament, wherein the medicament comprises at least two of the following populations of cells (i) to (iv):
(i) a population of lymphokine-activated killer cells (LAKs),
(ii) a population of cytokine-induced killer cells (CIKs),
(iii) a population of yb-T-cells, or
(iv) a population of tumor-specific T-cells (CTLs),
wherein the population of cells in (i) to (iv) are derived from autologous cells from said patient or from allogeneic cells from a donor.

2. The method of claim 1, wherein the medicament comprises at least three of the populations of cells (i) to (iv) or comprises the populations of cells of (i), (ii), (iii) and (iv).

3. The method of claim 1, wherein
(a) the autologous cells from said patient or from allogeneic cells from a donor are cells from a blood sample, preferably a PBMC sample, and/or
(b) the population of cells in (i) to (iv) are enriched and/or generated by cultivation in vitro, and/or
(c) the population of tumor-specific T-cells is enriched from the autologous cells or from allogeneic cells by co-cultivating the autologous cells or the allogeneic cells with tumor cells from said patient.

4. The method of claim 1, wherein the method further comprises administering to the patient one or more of the following (v) to (viii):
(v) at least one Th-1 adjuvant,
(vi) at least one IL-2 family cytokine,
(vii) at least one immune checkpoint targeting agent, or
(viii) at least one lymphodepleting agent.

5. A method of preventing or treating cancer in a patient comprising administering to the patient an effective amount of a medicament, wherein the medicament comprises at least one of the following populations of cells (i) to (iv):
(i) a population of lymphokine-activated killer cells (LAKs),
(ii) a population of cytokine-induced killer cells (CIKs),
(iii) a population of yd-T-cells, or
(iv) a population of tumor-specific T-cells (CTLs),
wherein the cells in (i) to (iv) are derived from autologous cells from said patient or from allogeneic cells from a donor,
and wherein the method further comprises administering to the patient:
(v) at least one Th-1 adjuvant,
(vi) at least one IL-2 family cytokine,
(vii) at least one immune checkpoint targeting agent, and
(viii) at least one lymphodepleting agent.

6. The method of claim 4, wherein
(a) the at least one Th-1 adjuvant is an adjuvant which is a danger signal and which is preferably selected from a STING agonist, a RIG-I agonist, an MD5 agonist, a TLR2 agonist, a TLR3 agonists, a TLR4 agonist, a TLR5 agonists, a TLR7 agonist, a TLR8 agonist, a TLR9 agonist, or a combination thereof,
(b) the at least one Th-1 adjuvant is administered once or repeatedly to the patient, and/or
(c) the at least one Th-1 adjuvant is administered systemically or locally, preferably by inhalation, subcutaneously, by injection, intravenously or intratumorally.

7. The method of claim 4, wherein
(a) the at least one immune checkpoint targeting agent is selected from an antagonist anti-PD-1 antibody, an antagonist anti-PD-L1 antibody, an antagonist anti-PD-L2 antibody, an antagonist anti-CTLA-4 antibody, an antagonist anti-TIM-3 antibody, an antagonist anti-LAG-3 antibody, an antagonist anti-CEACAM1 antibody, an antagonist anti-TIGIT antibody, an agonist anti-CD137 antibody, an agonist anti-ICOS antibody, an agonist anti-GITR antibody, an agonist anti-OX40 antibody and an inhibitor of indoleamine-2,3-dioxygenase (IDO), and/or
(b) the at least one immune checkpoint targeting agent is administered to the patient on the same day as the at least one of the populations of cells of (i) to (iv), and/or after said day, and/or
(c) the at least one immune checkpoint targeting agent is administered to the patient repeatedly.

8. The method of claim 5, wherein the medicament comprises at least two of the populations of cells (i) to (iv), or comprises at least three of the populations of cells (i) to (iv) or comprises the populations of cells of (i), (ii), (iii) and (iv).

9. The method of claim 4, wherein
(a) the at least one lymphodepleting agent is administered at least one day prior to the day on which the at least one of the populations of cells of (i) to (iv) are administered, and/or
(b) the at least one lymphodepleting agent is selected from cyclophosphamide, fludarabine or a combination thereof, and/or
(c) the at least one IL-2 family cytokine is selected from mature human IL-2 or a fusion protein thereof, and/or
(d) the at least one IL-2 family cytokine is administered to the patient on the same day as the at least one of the populations of cells of (i) to (iv), and/or after said day, and/or
(e) the at least one IL-2 family cytokine is administered repeatedly.

10. The method of claim 1, wherein
(a) at least two of the populations of cells of (i) to (iv) are administered, and wherein the at least two populations of cells are administered to said patient in a single composition, simultaneously or within 24 hours, and/or
(b) at least two of the populations of cells of (i) to (iv) are administered, and wherein the at least two populations of cells of (i) to (iv) are administered in a ratio of number of cells (i):(ii):(iii):(iv) of about 0.1-10:0.1-10:0.1-10:0.1-10, preferably in a ratio of number of cells of (i):(ii):(iii):(iv) of about 1:1:1:1; and/or
(c) the at least one of the populations of cells of (i) to (iv) are administered systemically or locally.

11. The method of claim 1, wherein
(a) the cancer is selected from melanoma, renal cancer, prostate cancer, breast cancer, colon cancer, lung cancer, bone cancer, pancreatic cancer, skin cancer, cancer of the head or neck, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias, acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioma, pituitary adenoma, Kaposi's sarcoma, epidermoid cancer, squamous cell cancer, T-cell lymphoma, environmentally induced cancers including those induced by asbestos, esophageal cancer, liver cancer, refractory or recurrent malignancies, metastatic cancers, and combinations of said cancers, and/or
(b) the tumor(s) is/are poorly immunogenic, and/or
(c) the patient is human.

12. A pharmaceutical composition, kit or kit-of parts comprising at least two of the following populations of cells (i) to (iv):
(i) a population of lymphokine-activated killer cells (LAKs),
(ii) a population of cytokine-induced killer cells (CIKs),
(iii) a population of yb-T-cells, or
(iv) a population of tumor-specific T-cells (CTLs),
wherein the cells in (i) to (iv) are derived from autologous cells from a patient or from allogenic cells from a donor.

13. The pharmaceutical composition, kit or kit-of parts of claim 12, comprising three or four of (i) to (iv),
optionally wherein the pharmaceutical composition, kit or kit-of parts further comprises one or more of the following:
(v) at least one Th-1 adjuvant,
(vi) at least one IL-2 family cytokine,
(vii) at least one immune checkpoint targeting agent, or
(viii) at least one lymphodepleting agent.

14. A pharmaceutical composition, kit or kit-of parts comprising at least one of the following populations of cells (i) to (iv):
(i) a population of lymphokine-activated killer cells (LAKs),
(ii) a population of cytokine-induced killer cells (CIKs),
(iii) a population of yd-T-cells, or
(iv) a population of tumor-specific T-cells (CTLs),
wherein the cells in (i) to (iv) are derived from autologous cells from a patient or from allogenic cells from a donor,
and wherein the pharmaceutical composition, kit or kit-of parts further comprises the following (v) to (viii):
(v) at least one Th-1 adjuvant,
(vi) at least one IL-2 family cytokine,
(vii) at least one immune checkpoint targeting agent, and
(viii) at least one lymphodepleting agent.

15. The pharmaceutical composition, kit or kit-of parts of claim 12, wherein:
(a) the pharmaceutical composition, kit or kit-of parts comprises at least two of the populations (i) to (iv) and wherein the at least two populations of cells are comprised in a single composition or in separate compositions, and/or
(b) the pharmaceutical composition, kit or kit-of parts comprises at least two of the populations (i) to (iv) and wherein the at least two populations of cells are in a single container or in separate containers, and/or
(c) the pharmaceutical composition, kit or kit-of parts comprises at least two of the populations (i) to (iv) and wherein the at least two populations of cells are comprised in a pharmaceutical composition further comprising at least one pharmaceutically acceptable excipient, and/or
(d) the population(s) of cells are in a buffered solution, and/or
(e) the patient is a patient suffering from cancer.
